SEQUENCE LISTING (40 sequences)

SEQ ID NO: 1 - DNA, 20 bp, Artificial Sequence
  Synthetic
gacgcacggagcctctttgt

SEQ ID NO: 2 - DNA, 20 bp, Artificial Sequence
  Synthetic
gtgcttgggctccactagct

SEQ ID NO: 3 - DNA, 20 bp, Artificial Sequence
  Synthetic
gcacggtagcgtagtagacc

SEQ ID NO: 4 - DNA, 20 bp, Artificial Sequence
  Synthetic
gcctcgagagacttccgtct

SEQ ID NO: 5 - DNA, 20 bp, Artificial Sequence
  Synthetic
gctgtctcagagctcggaac

SEQ ID NO: 6 - DNA, 20 bp, Artificial Sequence
  Synthetic
ggcacggagcctctttgttg

SEQ ID NO: 7 - DNA, 20 bp, Artificial Sequence
  Synthetic
gctctgttggttgtcctgtg

SEQ ID NO: 8 - DNA, 20 bp, Artificial Sequence
  Synthetic
gtacacactgccgaatgaac

SEQ ID NO: 9 - DNA, 20 bp, Artificial Sequence
  Synthetic
gtgatggagttagctttgtg

SEQ ID NO: 10 - DNA, 20 bp, Artificial Sequence
  Synthetic
ggaccggcgcagcgtctgca

SEQ ID NO: 11 - DNA, 20 bp, Artificial Sequence
  Synthetic
ggaaagcttaaatgaaacat

SEQ ID NO: 12 - DNA, 20 bp, Artificial Sequence
  Synthetic
gttagacgaaaaaccatatg

SEQ ID NO: 13 - DNA, 20 bp, Artificial Sequence
  Synthetic
gtgtgccccttgtcagttgt

SEQ ID NO: 14 - DNA, 20 bp, Artificial Sequence
  Synthetic
gccaaggcaattgacacata

SEQ ID NO: 15 - DNA, 20 bp, Artificial Sequence
  Synthetic
ggtgcgaacctatttcaact

SEQ ID NO: 16 - DNA, 20 bp, Artificial Sequence
  Synthetic
gatcgcgcaggatgagtaga

SEQ ID NO: 17 - DNA, 20 bp, Artificial Sequence
  Synthetic
gtggcctctgtgcagtttca

SEQ ID NO: 18 - DNA, 20 bp, Artificial Sequence
  Synthetic
gggtattgtatggaccagca

SEQ ID NO: 19 - DNA, 20 bp, Artificial Sequence
  Synthetic
gatgtcatgagaactacgca

SEQ ID NO: 20 - DNA, 20 bp, Artificial Sequence
  Synthetic
ggcagtttgggataacccga

SEQ ID NO: 21 - DNA, 20 bp, Artificial Sequence
  Synthetic
ggcataagggccaccggtga

SEQ ID NO: 22 - DNA, 20 bp, Artificial Sequence
  Synthetic
gtggatccagttcacttact

SEQ ID NO: 23 - DNA, 20 bp, Artificial Sequence
  Synthetic
gacgcacaataacctgaccc

SEQ ID NO: 24 - DNA, 20 bp, Artificial Sequence
  Synthetic
ggcacctgtggctgagctga

SEQ ID NO: 25 - DNA, 20 bp, Artificial Sequence
  Synthetic
gtgccgcaaaattagagaga

SEQ ID NO: 26 - DNA, 20 bp, Artificial Sequence
  Synthetic
gtatgcttgccgcaaaactt

SEQ ID NO: 27 - DNA, 68740 bp, glycine max
tatttatttgctctcaagtttttcttctgttttttttcctttttaagattatgtataagaacattggagatcttgtaaaatgagaacaggaatatttggaagcaaaactttcgcgtattccttaccacataaaaaaactatcgtattccttccgacagatttgttttaaaatcttactttgctttggctttctttttgctccattttctgttctggttctaggtaattggaaatgcatgcaaagttccagcaacatctgtttgatgaatgtgaaatacagttcaataaagatcaacagttagaacttataaatggtcaaatatgctttaaaatttacccatttgaaaatggtatggttaaacctttgagcagaagcaatatcaattattaatacagtgacgtttacattctactttcttatttataattttttatcttttggttgcagagataccagtcgaaaggaaaataataatgcctggttctttccatccattacatgatgggcatctcaagcttatggaagttgctactcggtacataaacttgtgttatttttttttttaaaaaaaatgctcaaatattttttataggtcaatctaatggactagtcataaaatgtgatttgcttgtttttttttctggttttattttaatatgcttgttataaatatatgcctttcaatgggctactctattaatttgcttttagaatttagtctaaaatattcgatggtatttatatttgttgataacattaatgttatttattaaagtagctactactggactggagtaaagaaatattttaactttgacattgacaatgaaatattctctttagaagttggcatcgtgtcaacaccatataatagtcaattcaccaaaaacaccataataatcaaatattatatctgattgggtagtgagtgtaaaatttacattatattgtagaagtttataattttcttttgagaatctaattaggatcagatatctatacgcaacgcatgatgatttatttaaacttagattcttttgttcataattttatttcaatatttctcttattaagaatacatctaagcagtatttgtggtgatgggtatccttgctttgaaatatctgcagtcaatgcagacaaacctccattatcagtgtctcagatcaaagatcgcatcaagcaatttgaaaaagttggtgaggttcttttttgctatcccagattctatattaaccattcataggtcataacttctaaatgcatgagtttcttaagtatcattgcatctaccaatttatgaaacggactctgcttccatttttatatccaagaatgggttcttacctttctatttgcacagtgaatactggacggaattcacatgttttgttttgagttcaaaaatttaggataagttcatatgacaatatcatacttggagtttaaggctatgtttggattaaagtttggaaagtaattttgtgaattttaatgcatgataaggagttctcatgcattaaatgaaaaaagaaaaaaaattgtttcttctagagtaaaagtacttttgagctctcccaaatttaattgaaacatgtactaattctgtttctgagtacttagcttgtcattgtttaatgtgccacctaaagtagcaaatggtttcttcaatggcaggaaaaacggtaattgtatccaatcagccttatttttataagaaggctgaactttttccaggcagtgcttttgtaattggggcggacacagcagtgaggcttattaacttatggaattacttacttcttggattaaatttacatgattttcaccttgtcatgttgatagcaaaaggccatcggggctggctaacagaagaattaaaaggaagataatctaaagaaataaagaaaagctaatttgattcaatttctgtctgacttcttctcattcattgcttgatatttataggcttacattgctattggtacttcacagactataacaattctgttgagggagcactatggctgtcccctaacagaatggagagcacgaatgcttttcactcttatgcatagtcattcaggtaagtgggcttattgtggttccttattcgtctgatagtttgttatgtctcctgtggtgctgcattgctatcacatgtcctgcacttttttaatacttcttgcttcattagttgtgtttggcatgtttctgaggttgcaacttcagttctgcaaaatgtaaagcagctcaatccctggtttctgctggatcatgtcactgtccctcatgtttttgtattttatccgtggacagtgcttttgcttttgaggaaaaacacgttttattcattagaatgggttaggaaccaacaattgaaataaaataaaaaaaagattttatttgactagcaaaacaagaacaaaaataggagagaaaattgtcctccaaaggttttctctttacaaaggattttcctttcacaaatagctaatgcatctacaaatgataagatttctctcactcttatccttcttttcatatttatatctaataaatccttttaactaactaatcaatatcttaactatagtaactaacttattaatactctaactatcttaactaacttttcattatttatatcctaatatccaaacagaatgttatttcagtcttttcagaaaacatattgcttatgccattttttatagctgaatggagtagagcaatctgactgtaatatgtttccctattccgttggttatcatcctgcagccctctggtttatgtcatgacatgacattcgttaagtggactggtttttgtgaaataaaattgtgtatttattttaagtttaaatatattttttcttataatttaatattttttaattttttaattttagtctttataaaatgaaaaaaaatacaacaacaaaaataaaaaaatatttaatttgagggatgaaaaagtatataaacctctattttaagatagtttcccaatatgtataatttttgtctggcacactgaaattgttatctctccttaaagacaatttgagttagattgagctaattgagacagggaatatttccaacagattgactaattttgaataaggacctaagtaccagattgcctgctttaactagcttattaggttgtaacttggcagggaaaatactctctactagagaacagaaactaagaagcgtgtgaatatcctttttgaaacagtataacttcagcagtaaacataattatgtttatatgaaatgttttgttttcattttattttctgtttccaaatataagaatagaaaatagtaaaagcagtttgctattgttttcctatgcagacttttaaaatcgagaaacaaaataaaaacaagatactgtttttgtaattaaaagtgaaaataaaaaatgaaaacaaaatattctcttaaatcaaactggccaataggaagaaacaagaagtgtctcattctctgtttctcgtttctgctgctctattgtggatgccaagccaaaatgaccagtttcagtacttaaaattctcactttgtattttctttgcagttaagaataagtatacttttattttttgttgcttcaatagattttattagtttagtattttcaaattcctacatgttactcttgcagcatcctaaatattatgatggtgactacagcatgatgctgaagatacttgttggttgtaaagaaacgggatgcactttccttgtgggtggtcggaatgttgatggtgctttcaaggtattactgcataagaaaagagcgtttctaattgtgatgttcctttttaagttcagtgttagtgaggccattttgtggtggtacaggttcttgacgatattgatgttccagaagaactaaagggcatggtcgtctccattcaagctgaacagttccgcatggatatttcctcacctgaaataagaaatagaaatcactaacactaaacaaaagggtttttatatttttttgaactatctttgtcaattgactcaatagtattttattatataatgataaaaaagaaaacattttgcacttttcagggtcatgatcattgcagttttgaaaggaactgtagaacatcttgtgatttattaacgagacgtattgaatagtcatgctaatgcataagacacgacacttgaaatccgaaggggagatcgatgtgtctgtaattgtatagtgttcactgtccatccttcgtagttttgtttttagtgtaaaagacaataatgtctctgcaacgttgattgaaaaagagggaagggtgatgtcgatacgatgcttttagttagtggggtttgagaagtttggttgtttgtatcttacaccgcaaggggagagattttgtagtaatcgcggggctctaattttattgttgcgttaaggttagacccctaaaattcaagtgcgagtgcgaactgaaaaacttacctttttttagtatgggtctttttcctttgtgagaaaaaaatatatatgtaattagagcatgtttggtatccagttgcaagttactcaaagatacacttagtgagtgtcaattgaaaaacctactcatgtaggtgaagtgtgagtttgtacgagtaatgataaaattgttttttaatgaaattaatttaattattttaaaatgatgttgtattagttgtttttattataaaaataagttaataataaaatttaatataaattatttaaaattgttttaactcaactaattttggattcataattttttagcataaaattaaacatgcgaaaatttactttaaattgcattaattggtattttaatgtagttttaaaaaatttaatgtgaaaccaaacacagagttgtcaatgtgaaagtgagtgtgacaatgcattgaaggtaccacagaattcacaattgtgtttaggacagaaactttgaaatatattgtttgggagggagagtgctactatgtttatggatttgatgcttttcttaacgatctaattttaactatagaagttaatttaattagaacaagttagaatttaaacttttgaattaaccaagacaatacaacacaacagctattattattattcataaatgtgttttataagatgtaatggggacacaatgtaagtatttcttttagtgaaaaatttaaattgcaactgttctccttgtaagaaatgaatccatgccacttaaattgtaattgaaaactcaactccaccttctttttttttctctttcgtttgttttatatatatatatatatattcattcagcaaggaataaagaaggaataaaaaaggcagggtgatttgattagatttcacgcatttagtttgggtcgtacaacgtacaatcaatcatttcatttcttagcatcctataccattttgactagaacgaaaaaaacagaaacatgtgccaaatagattaatagatactctcgtctcgttagttagttccttcacgtcacaattcagaggtgtaccgtacgtgcctgaaccctcaaccaccgcagcctccaccacatgctgccccacacccggtggcgcaggcagaaccaccgtagttgttgtcttgggaggctttcttggagttcttgggcacttcatctgcacagaagaagatgatagttgcaataagagagagggtcacacataacacaaaaacaagagttccaattccaagctcttcttcaccactcttcactactcccatctctttcaattgcctcgccatcttgtggtttctaagcttctgatctctgccaaagataaagagaaggtgttagctacaaatggttactgtcctttgcgccacgaaaaggtgacgtggaacaatgtcatgctacaaggaaaataaaaaagaacatgtgaatttgaatggttctaagactttcaataattataattatcctcactttttcttcctttttagttctacttttttttctattctctctttagatttttctcttcctttacctctatacatccatatataataaatataattaatctacaattctagagtactaattaacattttctctaaaataattaaggtggattgaagtagaaaaatgagagataaaaataaaaagaaaatagaaaataagtgatatcgagaaaaaataaaacaaaaaattgaagtcaaaataagatgggaaaaaaatatataagtagatgactataattcttcaaaactttacgcagttgaccgattaacttaatattgattccttcgcttttttaggacggcgaattaaaaaaatattcgtgtgccaattatgtgcacatttagttggcagccacacaagtctgaattgttaatgaacttagtcctaatcttatccttaaagtggcaaattaggatgtgaatccgagagaatttaatccttgcgttgcagctaaaatttgacacgacttaactaaaattttctcgctcgttcattttctttactttttctatattaaataattttagtattaagaaatttgctatatttttaatttctgaaacaagtgttattacttttcaattctttcttaataaaatttctgtatagtatactgtttttcttcctatatactactttcattaccatgtataatgtcattccaactaattcacatataattttttacagttaaaatttatgataaataaatattttttaatatctaaattatattttgaatttacgataaataatttatattatgataaaaaagttgattaataaaaactaaaacagacaagtgattaagaaaactaaatatatgaacaaagacaatgagagtgggtcgataaagaaatatattaggttagttattaaattaaattaaaattgagatatatatgaaatgaataaataaaatataaatgcttaatatataggattctgataagacatataattcaacgggaatgatagtaattcttaaaatgttcatttgaggataagtcctgaaaataatctaaatattgaagtttgaaaaagttataattttgaagtggattaaatttcaatagcttttctacaaaaatatcaaagttaaaaataattacatcactatttaaattatttattataaatctaaaatataatttatatatcaaagattcataaattatacatgtaatgtgattcatatatttaaaagtatttatttattattaattttaattatataacataaatatttattttataaaatgtgaaagcgaaaatactagcatcaaatattaatctcataaaaagttactaaagtaatttagcttaattatttaaaaaaatgtaaaaattattataaactttttaataatgaattcaattcttaagaataaaaaaataaaataaaaatctcataacaagcttatggttctacttgaaccaatgcaaaaggtgatggtgtttggtgtttcataaccaacctattccgatagtctttgcctcgggatccatcttttcatagatagtgatccctaccctatcctacctaccaatcaatacatggatcgtttacaacttcaggtgagtatattagctgtagttacggtaatttgcggcccaataacgcttaaaggatttttttttttctgaagttaaacgagtttaatctacatgcaaattcatttatttatataaaaaaataaaaaatatacattttaaaaaaatgtaaattaaaaaaataaagatattctgagctagctggtgaattattttggccaaactatagtaaaagattttataaacgaggataaaaaagtttgcaaaccaatccatgagttggcctatttaactgaaagcttaaatgaaacattgggcaatttaaatttaggcctacataaattggtccatttaagtattattttggtttgagaccaccgatatctcgtaatagaggcaatcattcttaagtaaagcagaactactgttgtaactaattgagtcatcatggcagctacttagctatggtaggcctagggcctatatctctgttacaaaaaaaatgacacaaacgatgaattagtagtttagccaacccgaaatttaataaccatacataattagaataaaaattaatgtttttttactataagactcctgcattaaaatctgtatagtttcgtaaccgacatcttcgtaactaactaacccaacaataattaattgcattgaaagtgaactaccatctgagacccaataataattatttacattaaaagtgaattgcacaatctgtgagcaatctattgacacaaaactgagacaaaacttctctgcaacattaggactataaattcagaaatgaccacctcggtaaaacatttatatgaccacaagtccacagctttcggcccccaattaattgtacgagcaattgttgctaaagaaagaaaagaaccactcactgaaatttgtgcacatatgaagaaaactgaccaaaaaaagctgcagcagaattcatgagcctcgtcaatgccaacaagagatgttacttgatacagtacattcactgattcacaatcctagcattcattcattcccaatcaaaaatcactttatcatacatcaaaatggcccaaaccaatagtcaagaaaagaaacaataaatacagtaccccatgtaattcatctactttcaaaacaacctattgatagttgcaacatagttgggatatcaataaatgaaggcaagtcagccagaaaataaaatagatcacgaaatctaacacaagatagatatcccagtatagtatagaaaaatagtactacattgtagccaaattgtgtgtgtgtgtgtttgcactctacacattaaaaagaaccttaatgagaagctggttgcaaagaagttctagtagagtgctcagaagcattggcaaggccttgcaacattctcaaaatctcacttgtgtcttccaaataatactttgccttgctaggtttctggccaacagtgcatggaaacacttcagcaactggagatagggttgcctttgcattcatgattaccccaaacatgtcctcgtctgatctgtcatctccaatgcatagaacaaaatctggaaacactcccttttgttgcattgttaagagaagacgttctgctacaatacccttactcacaccctgcaccaaaagttgaacaacaagaggagataatcaacccaaccacatttcatgaccaaatattagaattaattcctgtgatctacacaagttctttagaaggaatccaatcccaaatgcactccaattgttttgaacagaatattatggaacaaacagcagtcaactatgcaaacatagaacataacagggttgtcaaaacatggttctgggccagttatgagcaccactacaattaaatcaccttgaaggtccaaatccaaagatcctttctgattaaagaggccacacaagtcacaactataagatgtatataaaatataaacttcttcaattataaatatgtgtaacctaaaaatgactaaaagtggaatttttttttttactgacctgaggtttcacttcaacaatgtttggactacttttaacagaaacaggctcattggcaagaacactttccagatgatcaaaaagctccttagcttggcatgaaccaaagtctcggtctgcatactcgtaattccaaactagagcactttctttggcctctatgtttgaaccatcagttgtttccatatataactgcataaccggctcagcaatctgtttccactcaaaatcaggtactggaatacaagtatcccattctgcatttcgatttgtcctggataaaaaaagataaagttcagtttaagtacataagctagctctgccaaggaaaagtacttttcctcaacattctctcatacataaattgaaacagtgagtttattttgctgttaatgatgaaaaaaatccaatatgatccctcaatccaatcaaattttggacaaaaagaatcaaattgcatcattcttaaaattaacaaaataaacaagtaaaaagaaccattaatatgatttgcagtaaccacaaactcttcctgctcctaacacacacaaggaaagaggatatacctcacaaaataaccatgctctgcagcgattcccatcctttcacaagaagaaaaccattcagtaagagtctttctctcccttccacttacaatgaaaacacaattcttggtgtccctgcacaagatgttcaagatgctaacggcttcagcattaggtgttaaactcatcgacccaggctgcaccatagtgccatcataatccaaaagaattgctcggtgcttggtcctcttataagctgaaacaatatgttccacagatagctttctaaagtttggatccaaagcaatcactcggaagcctaaaccaaaaccaattccccagcatctcctcctcagatgatctctacatgccctttccagatcctgcaagaagctacgtgcccaatacgcaacatcatgtgtactaacatacctataatgcttctcatgccgcatctgcttttcagcctctgggaccatcaacgcagaatccatagcttcagcgacagaatcaatgttccatggattcactcgaattgccccacttaacgaaggggagcagccaataaactcagacaccaccagcatactcttcttttgagtaagcaggtctgtccctaaaatctcatctatcttctcatttccttgtctacaaatgatatattcatagggtataaggttcatcccatctctcactgctgtaacaaggcaacattctgcaatcacataataagcaattcgctcataactctgaagtggtgtatcaatcaagactacaggtgtgtatccaggccttccaaatgcattatttatcctcttcatcgtggcataagtttcactttgtacctcctgcacatcctttccacggcctcttgcagggttagcaatttggaccagaacaactctgcccctcttatcaggatgttgtaaaagcaattgttccatggccaaaagttttaagctgattcctttgaagatatccatgtcatccaccccgagcagcacagtttgatctctgaactgtttttttaactctgcaaccttgctttctgtctcgggatgactcatgacagattggagctgacctatatgaataccaacaggaagaatcttaatgcttactgttcttccatagtactcaaggccaatgtagccacgcttggattggtaagaaatcccaagcattctgctgcaacaggagaggaaatgcctggcataatcaaaagtatgaaacccaataaggtcagaattcagaagagctctaagaagttcatccctaacaggaagggttcggtatatctcagacgaaggaaatggactatggaggaagaatcctagcctcaccctgttaaatctctttctcaaaaatgtaggaagtaccatcagatggtagtcatgaacccacacaaagtcatcatcagggctgatgacttccatcactttatccgcaaatatcttgttcacagaaaggtaagcttgccaaagggacctatcgaatcgaccaccaagatcaggtgacaggggaagcatgtagtgaaacaaaggccatagatgttgtttgcagaatccatgatagaatttactaaaaagctcaggagggaggaacgttggcacacatttgaaagtgtcaagcaagtacagagcaacatcatcttgctcacttggctcaatctcttctttaagacaaccaatatagatagtttccacatcatccccaagaccatctttcagctgtaaaagaagtgagtcctcatcccatgtgaactcccaagtaccgttgtcttttctgtgtgcctttaatggaagctggttaccaacaatgatcatcctctcttgagagactgaggatggagtatcagagcaaacactgttgctggtttcatcatctaattcagacagtactccagcaacagttgccactcgagggagccttttcttctcacgactgaaagtcggggagccacaagaagtaagatctaacaagttagaatatgaccttgaaaccattttgatgatggccaaatgataagctttggtggacaagatgcagaagcaggcttctattaattgttcacttcacagtccttcaaacaaccataataaggatgaaatttcagaaatctgaaaaacaaattaaatgccatttatagcacgaaactaataagtccagcagaatcaataaaacctaatgttacattacaagaaaggtccataacaaaagtttggtatgttttctttacacctaaggatttaaaaaaattcaggtttggcacatagtactcatgagaaaagagcagagagggagataaatctaaaatctcacactagaaccccttcagctgggattgacagtagtagtatactttaatgacaaattatatttcgaagaataccacgttgaacaaaaaaggtaaccataataacaatttgagaaaatcctaaatagacaacatttgaatctatgaatagaattattaataataccacctaccaaaaagaaaaattcatccactaacactgagaattgaagcaacgttgaggatggctgcgtgaacacttaaatggatccaaacaccaagatatataaaaaatgcaagattagcccaccttcctttgatgaacgaagaagctcaagagaggccaaactagcccaaaagatgaatgaacgaatgaacagaagagcaagaaaggaagaaacttttctcaacagcaatgaagaaaaatcctaacccttgaaaacaacaagcaaaagagaggtgtgagttgtgatatgagagacagacagcaaaagattcttcttttctttctcttctcagtcacacacacaaacacttcacctgagtgagcaatggtgtcaactgggaatccctttttctcttacttttgttacaaaaacagaaaattcatagtgatattttttgcctattacacccaactaacgacaaattgggatgtctttatacaaagaacttgattctctcctcccctcaaatctcactatgcctatgtctctatctcagtgatgtgtgatgacatgactaccccaaaacagtgaactccaacatccttaaccacggttagttttttcttaatgaaagattaactccacctaaccctcctcaaatgtgacggtggtgcttcactgccaccactaagacatgcttaatgcacactagagcgcgtgtaagcacatcaccattcattttttttccagttccagcatatattcccttgtcactctcttgtgtacaaagtgggcttgtctggtttttttaggttcaaatattattctaactgactcgtgaaattaatttaacgcaaagtagtgtttaagtttcttaaattgtgatttggacatggttaatgcctgcttgaacagaattaattttattatgatttctggtcagagtcacataggaataactcattaattcttttgtgcatattgtttcgaaaatatttgaacaatttcattttaaaatttaactttagtaaattttctaacaacattcctaataagagttgtttatagaaatcttacaattaagaactaactcttgtataattttaccaatggagaaattcgcgtggtaattcttaaaaagaaaatctaattttaactttttaacaatttatttttagaaagaaattaattgaaattctcaaattttatatagtattgtaaggcttatatatatatatatatatatacaacaactctaccataataatattggagattctcttgaacaatgtactaattatgctgtattaaaattaaatgttagttttattttgtagaaattattgacaagcacgaccttagaatttgctgcagagtatattacacgcgcaatgttagtgaacaacattgcaacatgtgtctttgttaaaaaaaaaagtgtcaatggatagagattattttatgaacaaagtgattcttaattgttgttccgggtcacgacatatttggctgattttttttttttaaaaaaaaaaagagactgtagaaaaatattgctctttcaaaacaggctaatccaaatatgcattcaaatcttaagcattatcatgcaaatatttggaagaagagaatataagagaggggtaaattatgtaaaaacgtacgttataatagaatacaacagatttttttgacaggtagaatacaacaattacataaatgacataaatgttactaccattatcatgtacataagtgtgatgcaacatatatatcaatttatgtttttgaacattttattataaaaattacgtaaaataaatatagtttatggtaaatattaatttaaatttttgttaatgtttagactatttaagttattttcaccaaaaatataaaatctaatctgaaatctgttataaaaaaattattgcttacttatgatatttaatgtttaaaataattgtaaaatactcactttaaaacatttatatatatatatatatatatatatatatatatatatatatatatatatatatatatatgttttaaattaacatctttgaaaaaaggggtggttctttccttcatttcacaggtttatttttacacaacgaggacgtgcttctttaaaaaaaaaaattatggcgtcattgagcattggaaacccattttagattttctatcattatatcattttgtaacccttctcttttactctttgtgccgtgcacgagccttttttatttgacttccttttcagtcatggagcctcaagctaaaaattattattattattattttatcgtagttgcttatttgctgttcgcggtggagaataagaaattgtgacagagacgtttagttttaatgaatgtataattttaagaaactaagctaaaaattatcaaaatttagttcaatcaataaattttattccaacatctcaatcaaattccctttgtttgaatgacatgtattttttttagacccctaatttttaataatatatggtcattaattttttttccaaaagaatataaatattagttaaaaagacctcacctcaaaaccactttagagctaaaaatatattaaactaaccctattctttggtttgagcccatcatattgaaaacttcagaattcaatacttataacgaattagaatagatcaattattttatagaaccaaatcaatctaagactttgaatctgactcgttaacatccttaatttatatatttcttttttttcctatttagcttttaatccaaattgtaattattcaaaggtatgattgtaatttttccttgatgtgttgaactttatctgtccgtacatttcattattttttgtcactcttgatcgtgtgttaaaatgaagtgaatacatgcaaaagacaatttgatacttaagacaagacaaatatcatttcaatacagtaaaaagaaaataataaatgaaaatgaataattattttacctcgaaagtcgcactctttgtatatacgcatgtatagactttaggattcatgggtcatattgttaatgtgatgttaatcctttattatatggtaatattatcttaattcaaggattatgcctctaacattgacattgtcaactgtaataagatcgaacaatcctctcatgagtattgttggattatatgattatctaagggttctagatccaagtgggataaaaaacaaaaacattacttctcatacaaatcttactaacattaattaaacaaggcattaaatatatttaatttcttaaaaatataaaacgtttttaatatttactttatttagtactactattaattaggagaattcgtaggaagagtaagcaggagaatataaaattattaaagaaaacactagtataatttatctggatgtgtctcatgatcacacgaccgacgttatttttagccatacagcaagggtatctccaagaacataagttgttatatttttcttggcttttgccgcgacatcctttatatttaaagtgcatcccaaacttttattactagaaaattacttgtcatatgatttataaattggttccatggcaacttcaatcgatatctggaccgaacatgttagtttagaattcgtaacgtataacaaaatagattgcgtgatccaatgacttcaaaaatatcaccattatcaattactccaaaccaagttcaggttactacatatcatctcaaataaacaccacgcttgaggtcgccaatcatgcaacgtagaatttaattaatggccagctttattaatattaatcaaatgttttctttttgctgaataataatattaatcaaatgttttaccacaagtaaaagtaaaagcaaaaaaatcattatttaatattaattattttttaaaaaataaaacaaatcctgagaatacttttttatttagacgtccaatatattttcaagaaaaacaatttttaagaaagtaagtattgtaagtttaattgcattttcggtataacatttacaccaacaataaataataaatatttttttattggtgctgtttaagttttaaattttaataaccttatttttggaccagcttcgataaccgtaactttcccttaacatttaacagtcattaaatttacttatttaattattgctattacatcaattatcatttttctttctttctttcttctttatactccaataggtagacctagaagtttattagaagtccaagataaaagcaagggacaaatagtgaaataggagagaaccttctaagttataagtagagttgtgttggtaacatcaatgatttgcttgccacttttatataataaggctatcattattctcatttgtgttaagttgcttcttgacgttgagtgattgtgtccacctagccttgataccatttgtaatatttcaatcatcacggcttaaccaaaaaaaaaaattgaagaaaattaatacaatcattactgatgcgaaacttgctactcgatgtccattcctcatgcgtcataattagccttcttttttctcagttacaacttaacaataattattaactacttttcctttttccttagttatcttttactttcctaaaaaaaaaaaaccaaaacttttaggctggcaaagtcagaggaatgacaaagtctcaaacttatagcaaattaactttataaaaaagttaacgggctgatgagaagatttaccgaaacattatagaacaaattgttgaatactaataattcaaaatattcatttattaatggggtatttaaaaactatcaaatgattaattttattcgtttgtttattataaaacatatgttaattaagtatcaaattgtgtaaatttatacattaaataattatttaaaaaatagactacattatacttttggcctctatataatatccaattgtagtttttggtcttctttttttaattcggcaattttaacggggacggaaccaaaagtgtgttaaagggagaccaatttttttttacataattatttaaatatttaatttctaataaataattatttaataataataagttttaaaaaatattttttattgattttatttataaaattaaagatagggtctattactataaaaaaaatttaaacaaatatcaacttaaaaacttatttttcttatattttttttcattaatattttttatttttattttatttttttctttatatacacaaagggttctaattttgtaaaaaaaaatcatgttagactttttcatgttttttaaaattagtctactatttagtaaatatttgtttgttaatagttaaaattgtaacattaattattacataacattaaatagatggcgtgtaaaagtgtttagacatgtggatatttttgtttttattttttatttagtaatttggaggtataagggattttttaatttgacatccaattttttttttcaaccggattgatctattttataggtaaagtgatacctttcatactcagaccaggataatcaaacctagactttgattaattattatattattttagggtaaagtgtggataatctcgttgacgtatgcgttattttttccatgcaacgagttcaatgggaaaggaataaattaatagagggcaatgaacaagttaaattttcttgagtaatgagtacatatatatagaaaccatactcaagagtcaagactaatttacactcaggttgccttcagttcgtggcgtcatggtatgaaaaatcgtcgttagccacgatgatgatggtctctaaagtgtgttgaatgtctattttcagtttgcaaggtaaaaaagatcaaaatttctaacaaccttttgatacaacgtagaagcaacaaatggttgctgcaactcaaaccttgaatggtttgaaagggaggtgaattggaattttttttttttgttacaggtgaattggaaatttgcaaaataataaattatatctccaaaaatgctttctctcataagattttttgctaacgtgggtttatctagtctaatgtcaacccaaacacacaaaatggtgtcctgtaattaaaaaaaaaaaactaaaaaactagatttagcattttattaccatcagttgggttgaatgtgtttttctcatgaaaggttaataggcggcgcacgcacatccacattttaattatattttttagtttgtatggaaaactttaaataatatatcaaaatagtgttatagtttttatagttttaaaaattctgattttattaattaatttttaagttttctttcatgttaattgttttaaaacatcatatttctaaaacaaaaaatgcaataattttatgttggaatcttttgtttttaaatctcaactatttattttctcaacattttaatatttccaaatttagtcttttcctctcaaatttaatcttcttatatttttttgttcttgttatgtatgtaaagactgtgtataaatttctcataattgaaatatttttccattaagtattttttgtgtactataatgcataagacacctattaattgctttcaaaatgaaatacccattaattagtttctaaaatccatgagtttctatttctttgaatggtttggaatccatctctaatcatatacgttaagatttgttcataaaaaaattctttgaattagtttcataatcttcaccaaaaaaaatttgttcgtaatcttgagcaaggtgacatttaggatataccttatttcttggtaagttatgtacatctcataaatcttataagttatatgttttttttctttttaaatgctattatttaaatttaactaattttaattttaaattttagcataaccatatcaaaattagtagctataaaaaatgttagcttaaataatcatttagtcccaataaaatattcaattttttattttagtcctttaaatatattttttgttgttagtcccagtgattttcttaaattttaaatcttttgttgttagtcaatgttgcagcaacaacgtaacgttattaacagataacgtaatagaaatatatggagtttattgatttattaagccattaaagagagtctgatggaataaatatgtgtaatttttttgtaatttgtttttaaattctcttatatttacaataattgataaatgattaaaaatacagtttataaaaaaaatatgtagttattcaattagactttttaagaatataacgagtcaatatcgtcaattaatattattatttaacaaaaaattcaattttgtagggaaaaaaatctttgaaagactaaaactaaaaatagaatattttataagcatcaaattgttatttaagctaaaaatgtatcttaattcaattactttaaattcataattttcccttaaattcaaaagtcttgtatcattttaattcataatattgttaacaaataatattgttataaaaatattttattttaaattttattaaaattggaaaactcatataatacacgaaagacttgttgatacatttgctaaaagtaaaatgctcaggatacaaaaggacatactcaaaatatgaaatttccgtaaaagaattgctaatataactaaataattttcaacgaataattaactgaacataatgttatatctttggtgttaattatcggtacaaatttatataatcagagatggatatttcgtcatattccaaagactgaggaatattcttcttcttatttttttttccgagagtcaaaaccaagattattgacttgtgaccaaagaatactaatgaaagagacgagctaactccccaaaatgatgggttgatataatttagatctatggtcaaatcacacttactttgcaaaaccaaaattatgagctaactccccaatttaatttatcttttatactccacatatggtttttcgtctaaagatgatttgtccatcattaaaatttagaatattatcaatttgttgcttaagtttttatatacaattttaatagttgaatgcattcacgttcacggtataaactaattattgttatttaattataaattattattggtttaatttttaagataattttataaaagttaattgatttattatatatggtaatttgtaatgaaataacagtataacatgcataactttttttctctaattttaataattgtcataaaaaaggtaaaatatatttggagttgctatataatttaaaaaatatcaattttgttcacataaatttttagtattaatttggtttttataaaaataaaacatatttttattatttctcagtcataattttgttaaatgataatttaatttaatgtaactaataaatttatacgtataatttgattataactaaactaaataatttatgacagttaaaattttaaaataaattaaataattttttatacttaaaaactttcaaaaattgtaatttttttgtcagttcttaaacgattttcttacactttccggtaagattaagggaagagatgaaacataataaattttaatatcttctaaatatgctcagaaaagatagaaaaaaaaagtaaataaatttattagtcatgtaagtcaatgatctaacggagttataactcaaaattaataaaaaatacgttttattttcgcatgagcaaagtaatataaaaatgtgtacgaaaataaaattcccactttttaattttataaagattctaaacatattttatgctaaaaaaatattgtcattcttttacagaagtcgctgcctgtaagaaaaaaaaaagtttgtggctttgatacgaccttaatgagaatcaatctaagtgttgaaagagttatttagtgttgtaattctatcacctgtgcatgtcgagtattcaacggtggaagaagttacatgtatcaattcctagttgaaaatgttatgatttgaatgaccgtaactatgcttaaagttggggtttacgtggcttaatttgttcccctatagaaaaagacatctaactgtctacaaataataaagagttgaagtggggaacccaaccccaccaattattgttacaaaatgattctccttcagctaccgaaaatgaaatagtggttataattgtcatcaaaaactagtgtaaaaatataaatgatatgaaaatagtgtggtttttaagggagtgtctagattagattagtggtgatttatgaaccgatcttctttattggagttaggctgttggagattagctcgcgtgagttaagtttataaaaaaaatatagttggatggcctttggaagatttgacctaaatgttgaatgaacccactcacacattcacacatgaactctctattctttgtttttgtatccgagaaaccctttagtcatttaatttatttttattagtataaaaatcttctaacttaaactaaactattgatgtaaatattgcaaatatatattttatagtttacataatatattaatagtatgatccagtgacattgtatattaattttatgtaccaaataaaaactttcgttcacgtcaaaaaataaaaaacttctgttagtgtgacattatgttattagcataataatactaattattttattttgttggataaaattttaattttcaatcatgtgaaatgatatcatataattcatggaatcagtttgaacctcacctagtggaacaaaattttattgtttttgttacataaaatttacatcaatatagataagtttagtacatggattttttctttacaattaaaagaaataaaaatgtattgtgcaacaaaaaacgagtgatcattacttatatatttataagatgatattagaatttattataacaatctattattgggcctatagtttcacccgttattgggccattatttttttaacccaatttttaaaactgaggcaacatgagaaattcttttggatagaaaaaatcttataatccaaacctattgcgtttagattttatcctaagtcaaatatttttttaaaatacgtttagattttggacaattgggtaatctagtatatattggttaagtcaaatatatttcgttttctctttaaactgaagattactttaacctatatatatttcctttatggtatatgttcatcaatcctgtttttcttgttcgaaacctaccttcttattcatgccagtagacacgtcctttttaatgtaattttgtgatacgccaattgaatggattgattgagagatacgtcacatatcttaacatgtttaagtataacttgggagattttttagttaaaagtttaaacataacctttaataactcagaaacttgtcccaagaaattatgaattttatagttgtaatatattttaaatgatatgaacattcaattattccattaccctaaatatttcatagaaatatgttgttgcaagcacttgttttttcagagaaataaatttacaagcattattttcttttacatacttaaatcaggagatagatctaaatctaagtgtagccaattagtggtacagcttatattatagtgtactttatttcgtgcattatattgcatatctaaacttcatttcttcttcttttctttcacaggtccccttggaatcttggttagaactttagtatccaatgtgttgaacaatctctgcacgtcttggtttcctttgcttgctagataattcacctcatattcatatctctcatacataatgggaagagtcaccaggcagaggaacactgtaaaacaaagatatagcagatcaataaattgataaggttcaattaggaaggttgatacattgataataaaattgaattgggatctcactgatatatagaagattcaaagtggtaaaataattcccaatagctgataagatccagagacacgcaattgtctgaaattcaatgacatgatgttaaccaacaattaattaagaaaatattctgacaagtaatgtgagagtaattgaaattggtttctcaataccacaaagaagagtgtgaggtctttcccagttgaaatgtcgtaaaatctccttaagaacgagttgagcttttgaaacaagaatctaaaggtgggttcggggatttgaaaatcatagatttgtggcaggttcctgcaaagaagcttattaatttatttgctccacaaaataaagaaagattataaattaatgtatacagagaataagattacagtaccatgtgataagtccagctgcattataccatacgaataggatgagcataacggccatgaggatgtgacaaagtagagtaagaaaattgtattcgaccacttcaaagaggaaccaaatgatggagaaccctgctaccattgctgccgataatatcttgtctttccatagcaatatatcagcaactgcaaaatcagatcaatctatcacaaaaaatcttttgaaagaaaggtacgtttttctgcatttggtctttggaatataaatgaacaaaaatgcatccaatggtttgagagaaaaccctcaaaacaacactttggttaaaagtaggaaaactttctaccaatcaatgtaaaccctcaaaagtcaaaacaacccttaaaagtgtaaaagtacaggaataaaaataaaaagtaaaagtacaataaatgttttttttttctaacaaaaaattatcactttaaatacttaaaatgaatccaaaagaaggatgttaacattttccttaaaattttcagatcctctatattagaatttagaaacataaattaagacgtaagaagaaaaaagtggtgaagtgttgctaaactatcttacgctttcctccgccaaggactgcatgtagtggtctttgacggtctaacaatcctggcctctgtgcagtttcacgggaacggattggcatggttaccaatttgaatgattgaaattatcttcgtactttgtgttaatttgttggttctcctgaaccaagttctagcactgttatgattcatgcgtcatggtgccaactatatgtaatgttcgaaaggatatttggggttctaagctttgtaaccaagaaatttaattaactactcattccatggaggaggaaaggtatggctttggcatggttttcaagttttacttcctcgtgcccacacatgatttaagatacattattagcgtctcacatttacccttatctatgtgtctaatgctatgttgttgttttttcttttttagagacttttcgtgggaatcttatcgacacctgagaaggacggagacttgtcatgtttgtgctcgacgcaaatatttgacatatgatatacaaaatattacaattatgatttcaaattctatatttctttcaatatagctgttatgtatcacgctgcaattttagatcgataatagtaataacaatgtcgtcctgtgtgtttttcacaatcattcaactcataaaactagtgggaagaattcatagagagtaggatatgctcaaacggcttcatgatgtgctagtatttaataaaggaccaattatataataagataaataatgtaatagagaaaataaagaaaaaataagttataaaagattttatgaatttattatatatataatagtataatatgaatcatcagttaattttcgtgatggaccatgtatagtcatgaagtgcaataccggtatcagtttaatgtggagactgaagagttattaatttggacgtgcttttaaactttgttaattttaatatctttgaactcaacccacattccatgacattttgtttttgacaaacgacccatgacataagattgatgctataagcgtgctgctattttcacccccttttttttctttcaaaaaaccaatgaaaagttcaaattacccttctctcaattctcatctccctacaacccctacctcctttctcttcctctcccctcctcactttctgaattgtgattccgcaaacacccattccatcccttcttccaggtccatcggccattgtttccgccaaagcttcacatcagagtcggagaagttcttgctatcttttattgcttatatttaattaattaaattattttttataataagttaaacgaacaattaagtctaaggtttaattaaatcaaaattaaaaaataaataaataaaacgttgacaaaattaaatattgaacccaaataaagattttttttaaataaagagaccaaatatttttatttaaaaatgatgagactaaaattgcatgttagaaaaaatagagattaaaattgtatttaaagttatgttgaacaagacatttgattattcagtaattaaatttttttaatagtttcttgctagtgtctaatgtttattgaatcgttatttgaattagtattttttaaaacattaaattttaactttttgtatttttttctacttttattcttaatatatttattcattttttcttatcatattttttaaataaataataattttattattttctaacattttacactttacaaatactttaataactaattttacgaaatacttctaatttaataaactaacttcgaataattttctcaaatataacctaagtcaagaaaaattatccataaaatcaagcggttttgatttttcacttttttacttttgtaaacataatcataaagaaaactcaaatgttgaccatctatcatgttgcacatttcgcatcgccgaaagtcattcggccttcagcttgagtttgaaaagctccagtctccaacacttaaaaatcctaaattctttatgagaaccaaattactcgagatcattagttaattatgaaaatattatatattacataaatttatatttacatatacaacatttatcttcatatatgacaataaaaataaacaaacgtttttacagatttgacattaattaatcattgtatatatcaatttggctatttgagatgttatgtagagcggggatgagatttgaactccttttcgtggcttacctgtgatgagaaggcacgctatcacagtaattgttcagtggcttatatcatgtccttcgcattctgtggtattaggtgccacctaatttcttagtaatttatttgatgttcattgatttagcttaatatttcttataaaacaattatagtcctcgacagtcaacatgataaccaatatgcacttatatttaatctattaataaaactttaattgctcgtaaaaaggttatgaattatgatactgtgtataaaaagaacctcgaggcttctttttcttcttcttttttcaaataataatatttctataaattaggctctctagtctcttgtctgacaatgcctaaccaaactctcgcaaacttatgggtgataaaaaatgtctttgatctaccaagagctaaacataaatcctctagtaaaaaatgtaaacaacccgtcaaaaaatggagggtaaagtaaataccaatacttgttaatatagccacgcgagaattatttgtttatgatttgtgagcttctacagagcgggaaagacattgaagttacatttttctaacgtaacactgttaacattatcaagctcgtataataatatgttcattttaatactagtgcttttttatattgatgcaacttataaatgattaggcagacacgtttgtttcataatgatggtggcaaacctatacggctataaccccctagcatggcctgagtcaaataactgaaatctattagcaatgaatacacaataataaagcaaagggggtcctccgcaaattcatgatgatagcactaataatccccatccacataacataacacttgggcattgatgaaattggttttatagtagtcagcagccttgtcctaaggtcaggtaatgtttttgtttattgtttacagatgccattatagcgaagagagccacgcccgtatgcatccctgtgtgtcttaatcttaattaagagtctcacgtatcacctctcaattaaattttctccttcttagtctctctttgaaaatgaagacctcatatgcaacattgtgcccttctaggtcagaatgagttgtgcatggcggcagtgacgtgtaacgcgtagcagctgagtgcatgtgccagcgccatcacgtcctcaacccctccttcagctgtttgatgtagcaaactgaaggaacagaggccccaacctcaagaagcttgttaactctcctaatgctaggaaggtagctaatgcatgtggttccccattccccacttgctagaaacaattgagtatttcacaagtctctgcttggtgtggtcgtgctacaacactgtgtgccccttgtcagttgttggagaatttgatcttgtaccacctctgtctgcatctattaatgaatatgtagtttgctgattatgagcgctcagcttttacttggtccatcgtacaaaactacaaataatttctctgtcacgactctttctctttctgtgagattaaattataattatatctttttacataaccagttatgacttatgagacaatcatttcatttcctgctcgagtaataagtagtattacagtaaagctattcgtacttcaattgaatatctagaggagaaatgttaactgcattcctgcttagtttttgtgttaaatctaacagttttggactttcctccaattataaatcattgttaatatattttttatgataattatactaaaaatctaacagttaaaaattgtcaatgatagccttgttttttgtctctatataaaagtactcagatgctgaacaatgtaagcacaagaaaggtgtgtaaataactagtgttttgtctttgaattagtggccgataaattgggaaaatgaataagtaattactaattagtggtaaatatatagttcaacaaaagaaaaggatgagaagaacaaacttgggtcaattactttacattgaggtatttgcttaatgatatccgatcgtctttctcttcttacgccgccacattgccttttgtatctcttttaattataacggataagagttaaaataacagtgataatactgataacaataacttttaaacattctgatgttttaatagaggaggtgcttaaaattgaagattcctctttgctgaactaactggtacacatttttcatctttatctttctagacttattgtttgaagggaaaagaaaagaatatttttttctataaaaagaaaagaatatagtaagagtgaatggaatgaaacttaaagaaatattttattttttatttttttccctttttataaaataagagaaattatatagttagtggaaaactaaacgatgaaccaaggttatgtttgtatttttaaaaataaaatattatttaaatttattttgtgataattagataaaatagaagtcataatattattttagaattttaaaaaaataattttaaaaggaaagattaaaccatagaagtttttcttcttcttcaaaacttatcctaagttatcctaaaatgttcatgaaatagttttcctacaaaacccaacagcaatatctacccagaccacagacttgcctttccccagatatccagccgaagacattgcctgtctccttcacttacctcccttctttcactctcaaccgtcagaaaacatattgcatttaaataagccaacatatcacgcacataataatgataatacagtaataacaatgcaatgccaaaatatctagatatttaaaatttaggctccgacataataattgataaaagccaattaataccaaattcaaatatcaagatagagtaaccttacttgtataacaaatttttgcaacaaagtttctgctcattcattttctatctccaacactcgttatcttatcacacattaagattactcatcaagatgataaaaagagaaaaaaatatatcatcaccacaaaacacgaaagtgagagcctagttaatcggcccttcacttttgaaaactcgagtaaaaaggctttgtaagaaatttacaagtttagtaagagcatctccaattaaatttaagaactagttcaatactttatgttatgatcaccgttggaggaagctaacatgatttccaactgtaagaactggttcttatataaacaattagttcttatgattaaaataggatattttatgagatagatgataataagtgtgacccattttaagttcatagttgattgcatcattagaatagaaaattactatagttcttaaacttcattggagatgctcaagctaatttctactacaacagcacatgcaagtgagacacaaaaagcatgtagctggaggatgccaaaactttaaaactagaagattggaatatattaattaaaccaccaacataatactaccatgaccatctatcaatatatttcaggcattctcctattacagcataaatactatatattcatccattatccaaaagcaataagaggaccaccaaaattgagtttaaaaccaagaaataaaccattcaggccttatcagagtcaggaccttggtggtggtgttgcgtggccaatccaagtccttcattgtggtgcttctcccagaaaccagtgtcagccttggagtgtgcccaatagtacaaaatggcggtcaacatggacaacaaggtcaaggcagctccagcagcaaacacacccttgcgtagggtagcacaagacaaatcgtgattcacaaaataccctctgtactttgtgtggtatgcattccttgcagaccctgctaacagacacgcctctgccgccaaaaaactaatcctgatttcacagtagtaattattgaaacaacaaattaataaaatatttccaatttttcaattcaatttcgtcttctgacagcaaaacgcacaatcatttcataactggtgtagctgaagtgaaagtaaaagtctatctagtaatctatgactctaaattatgttcagttttttgttactaaaataattacaagataaattcgtagggatttgattatttaaaggtgaacttagcagtgcgtgtgattaattgagtgataccatgagaggatgaaggagatgacggcggaggtggcggagcagccagagacgaggcctttgccgcagcaaaggcatcgcgtgacgccgttaaggacggtgtggctgaggaggaggagggcgacggcggagaggccgtagacggtggaggcgtcggtggtgtaatggcagaaggtttggtcgtcgtactcgtcggggaccactttggcctgaaaaggaatcaaatttgagagctcaattgcggaaaccaaagcgagaggggggtagggttacctcgctacgacgtcgttcggcgccaacggcgaagacgaaggctatgagatgcagagcgatgatgaggactaaaatggttacagaaactgccatgatttctctctttctctcactccactcactgccttttttgtgcttatgttgttatgtctttagcatcttcacactctctttagctccggcttttcctcagcctaattcttcctttttcttttattcctacatttcaacttctttttcctaaattctcattgcattttcctttttttattttttaactcttttatataatgtatgaaacaaaaatactaattattttcgtcaactgttcatcatgttcgaccatatcccaaaactcaaaaaacatttttataaatacatacaaatttatttaccaataaaattttcacatcagaatttaagtttatgtttttgtgagatatgagtttattttttatttattagacaaacctttattgattaaatattattcttataatttttttgtataattgtcttaaaattgtaagtaacataaatagtattgttctaattattaatgaaaaaaatgtgtggaccattgtttttattctttatttgtcgttcaatgcatgtttggattttggttaaattattcataatatcgttaaaatactaattaatgttattttggataaatgtttgcttattttatattctgtttggtttaacgtttctctatagataacacatgtcaatgagtcaatcttaaatagctttgtaactgtattggttaggataagtatctgcctatccctatcccataaatccataactgataagttagctacttactcccacccatccatacacactatcattataaatacacagacatttctatgctgtatgtatagaaatcttctcattctattattaactcagtaacttgagaaatctggtaagaacggtaatacttttgcttttcttgctcatttggatttgactttaccaatttacataccttttttaagcaaatccaaacatgcatgttgaggaattgtttgagtttaaaattaactttggactaaaacaattttaggtagattttatgttagtttaatttttttaaccaagtttaaatccttttaattaagatcaatttcgtataatcgattcaataattttatgaacaattacgcagacaattctgtttaataaatgtgtaaagtgaacaataaatatttaagtgattaaaaaaagtagtagcttttcttttttgttttgtttaggctactttggcgcgaagttgagtttgggattggcatttgtggacgctagtgtaaaagttcgttttgctttgagttaattgtttgaaaagaaataatgagatattccctaaaaaatgatgggtttggatacactgtgtggaagtacagggaagattggtgagtttgggcactatactgccgtagactgaaatcaatcacgtagctgtgacccaaacgatggtcgtgggacccagctagataattattattcaagtgatttagggtgggacccatttactgccatcgataactgtgagtcaaagcgaaaatagctggaacacggttgtctggtcactggattgatggtaaggttgccctgttttttaatcacgaaaatgacatataaataacaaaattaataagcacccaaatatattaggctccgaaactagtatctttaataaataaaaaataaattaatttctgtgtattatacagtatataattaattaaagtattatacattacacacttgacttcattacaaaagcttgtcgtggacactgcaaagttttcacagtgtattaaacttacaattagcaaatgcaggaagaaacgccttgcaaatagtagcaatctcttgcctgtggccacatcacacgccattgtcgcctactgtctttgttctctatgcactgtaagttgtaacaagtatgattgtcggtgcggatattgtactagaaatctaattaatgactacattttatatcctcgttaacaaatctaataataaattaagatataaaaaatgatgagtgagtttatgattatgttgagcaaaagctgaaaacctacctcaaagttaaaaattaaaaagttaatttattaaattataatatttgataaaattaattatttaaatagctaaaaaaataaattaacataaaaataataaaattatcatttatttaaaaatttaattagaaaatttgataactataagctaaacaattagcctttaaaaatatattattttagttttgttcaaaaaaattaattaaaaaattagtcagttggaaaattagtttattaaattataaataatttattaaattataagtgtttgataaaaatagttattgaaatagttaaaaatataaaatgacatatttaaaaaataataataaattgaataaacatttcaaggataaaaagaaaataaaatttaaaatttaaaaactaaaagttatcattgtaaaaaaattgtactttgtttaaaaaatgttaaaagttagtaagaaagattatttactgaataatcaaaatagttttttgggttagttaaaaactaattaaaatagtttgtaaaagatagtcaatatttctcataattgagagagaaaaaatacaaccatttatttcatatctaaaagaaagaaagtattacattaagagaaataatatttgtacaataaataatataacatattttcaaactaaaagaaataaatatgagatgtaataaataatttgacggagcgacggaggaaaaaactgttgttgtataaataattattgtacgaatagcacaactctcattttaatacttaagctcatggtgaaacttttaaagaatacaaataaaaaataaattctatttattaatagccatagatgaaaaaaattaacaaatagttctaaaagagaaatattttcagagcatggaaacataattaagcaattttatactttttagtatactaaattataaatgctagtaaaaataaattaagttaagcaaaaattaatttatgtattttttcatactttaatcttctaatataaaaaaagaaattatttctatgtttacctaaattgaagaaaaatatttatttcatttttttgtattgaacataaaatattttttaaataaaatacttttttacataaattgaagaaaactgttattattataattttttagtacatataattttaaaaaatattaacaaaaagtgactaatttcagaaaatatttgctattctatttgagatgtggtgcgccagaatccatgatcgatctagtcatttgcagcattgggatgtgagttcctaacatgatgagcagctggtgtagtgtttctttttgatggataaccatggagtttgtagtagacttttgcagtatgtataccctctttgataggttatgtgggcctgttggaggaagaatagttattggaacctgtgtgtgttataacttctttatggtaatgtgatttgactttgtgagcagcatgactcatcccaggtgagatagttctcaaaatcagtgatgaggtcatgtatttcttcaaagctgattggatttatctgataggtgtgataattgcatgcaggatcgatatcaatggtcctgagcatgggtgggggattcctcgtgcataggtgttgttatttcttttatttcaagtaaggctctatctaaacatcgtcacaccttgtggcttttgccatgtttagtgttacttataataatattttcctttgctgataaaaaaaataataaaaatcactgtgatttaaggctcggctggttttggtgtcatcaacaaagtcaataaggtaatagccaacaagagagtttaatttagaccctccgagcagtataactaaatccattgagcttaattgaacttgagtgtgattactgattgagtatttatggtagggatattggtggtttcatcagatgaggaagtcatggtggaagaggtatgaatcaaatgctgttaagccaaggcaattgacacatatggagtaaattatcatgggagttctcatttttatttctttgttattattatagctagagtaaatttgataagagataagatgtatcctaatctagactaaagctgcaagcctgcaacaattagataagatctacgtttaaattacaagataagatattaatagataagatcttcatttttagattacggaatcatgtttatcccttatctatttagttagccataattatgatgtgttcagttaggattctatatccatttagttgcagtagcttcttctcttgttatataataaatatgtgatgagttctttgtacctgcatggagccgctcgatatggatatggatcctgacttcaggtatgctgcaagattaattaatattcatttatggttttcttccattttcttatttccttgagccaattacctttattgcttgcataatgtggtgttttactcggttggatgctttgttgtttggttctttgacaggcaattagggaaggatagtgaccgtgatttcatggactcaactagtgattacggtacatgagggaacacaggaatcttccttatctagtgatgatggggaatctgtcaattctcagggttactttttttagtatcttgaactggagaccctctcagaagttgtgatttacaagcatcaagttggatatctgtagcctgctttgagtctttgacattcagtttttacagatgaacaagggaatgacttgttcttctttttgcttgtatgcatccaatttgcaggataccaaccgatccaacaatgaaagatcttggattcttgatgccttctttctgacatatcattcccattatgaacccgtgggaggtaggaggatttttgcttcatttgtgcccgtagcggatttggttttataacaaagttcaagaaaagatagtagaacagtgtattacattaaacatgttttatttgaagaataaatttcctatttgagattgttgtgttaaaatgttagagcattaaatcattaactagtttgtcatacgttgctatatgtaaaacaacaactaagcattttcccactggccctaattttctgtcttgtgtattatggcttatgtcttggcacctgttagttgtgatctttcttccatagttttgttatttcttaccactgtgcattattttattgctttcattgattttagcaaatttattagcccatttttgtaagatatgatcttcttgtgtcttgtttaaatttgtgaaatgtcattttggattagttggggttctttattatttctactacaaaaacttctttttttatcatgggaacatgtttctatttaagcatacattgccaattattggttcatagtaatgaaattattgtgagatgctgcatctgataaccatagtctgttttccactttgcatcagctatgcattttttctttctttcaagttcacaaagaaagagtatcggccacggtgccatcacatcctactgacatggacagtgtccataaaatgtcttgcttcatacaagttcaaaggatctctggattcataatgattggtggatatgaatgtcaattagctagctctcttctgtaggcaactggttaagggttcctttgctgacaactgattaagactgcttcaggtcagccgttgatgatgttaaggtgaccgtagctataaaatacttacttctgctgtttaagaactgaaccttagtatttgccagtccctcctttggtccatttcttagatgtcacacttgctggaatcagagtaaatcacaaattcaacagaaggaaaaagggaaaaaaaatggtaagctctctgtcctttgcaattccttgcattgtttcctgccgtaataagtggaagaatcataaaagtaatggtaaagcagcgaagctcggcagagttgaggctatgtcagcatgtgttcatggtggatactggatatacagattaacgagccagcttcacaaggttggttgatacatgttatgaggttttttgtttgctaacatcaatgagccctgcatggattgcttcttgttgatttccaactatattaatgttggtatggttttgttaaagagaacaacaggctacatttttttctgtcctggttgggaaattttactgtataggagctaggtggactcacgtttcttagctgcattacctgtagaactcttctgttgatttactataccaatgaagggttccttttctttcgataatggtaaagttcatgttgatagaactaattaaaaagttcatctgtatttgagaatgatattttataagtgagttcagaagaaccaagttcatttgtttaacctaaactgcaagccaatacagctaaaagtacaatattcctatatatggttatccacggtcctacaaaaacttgataaggtctagttattaagttactaatagaagttagtaatactgattctaatttatataaagttcagtatttatagcatatgcctatggtactttgcccattcgttggggatggatttggccattttggttcaatatttatactagagtgaaagggtttttatggtattgtaattgaaatagttgtgaattaaaggtatttcaattctcaagtctaacattaatctttttaatgtgtaaagattactccatgcctcttctaacaaaatatattttagataaatgattaaaaatcaagttttgattgctgtatttaaagaactcaaatatctgttagatttttttgaaatattttaggaagtctcattgattgcagacacggccaaatggcaaatattaaaaaatcgaatgcatcactactttatgaacttgtgctagatgaatatttgtcaatcgataatgtattggcttgtaggattgcggacaaccttatctaggaatatcacactttaatctttttttggcttgtagtttgggtaaaattaacgccagtggttttcaaagttttactttcgttgctgatatagtagagtcttcagcatacatgtcaactttcctataaaaaaattacgaataaaaaaaccttgacatttccccctccgaacagtactagtttgtttgcttaggtttgagatgatttcaaacagttatctgtttgttgaaaggaaaaaatatgcgccgagtagaatttgttaagagtaactttaaccttttcgtacagtaaaattaaatgtgaatattattattatttaacagcaagtcaaaactgattcttctaaactcaattttaaaatatcattttgaaaacagaaaaactttttaactggttcagtcgacaagaatttcgccaatgactaaaagcaaatgaacccttcgttggtatctaaaatcaagaaaaagttctataggtaatgcagcaaagaaaaacaagtataagaattcaaatagagttgggggcaattctagcatgaatacatctaacaccatatccagtaaaatttctccaataaattaaagctaaagtccaattatagaacatcaccaggacagaaaaaatgtagcctgttctcttttaacataaccacaacaacagataagcaactgatggaggactcattgatgttagcaaacaaacaaatcagctagcacttgagaagctgcttgttaatctgtacatccaccatgaacacatttaagcctcaacactgcctcttgtccaaagcttcactgctttattaccattattgcttttctgattctttcacttattacggaaggaaacgatacagaaaattaccaaggacagtgctaaacatcttttctccatttttcatcttccttctgtcaattatgttattaactccaacttcatcaagtatggccatctaagaaatgaaccaagaggcactggcaaatgctaatgttctattcttaaatggccaaagtataatattacagtcaccttaacatcatcaacggctgaaaaactgaaaatctgggtgactgacttgatgcagtcttaaccagtcatcagcaccctgcaaaagagaccgctggcattcgtgtccaccattaggagtccacagagatcctttgaacttatatgaagcaagaccaaaaacaggtaaagacattttatggacattgtccatctcagtaggatgtggcatgggagcctggactctttgtgaacctgaaaaaaataatgcatgtctcaataaatcaactggtgcaaaataggtaatggatcatggttatcacaagtggcatctcacaatttcattactatgcagtatgcgcctataattggcaatatgcacttcaaaaagaaacatgttgccatgattaaaaaaaaacacaatatttgtagttgtagtagaatgatgaagaaccccaactaatcatacatatcaagtaagaagaaaacaatggaagaaaaatcacaattagcaggtgccaagacattagccatattcatataatcgtaaaccaaggaagattgtgaaacccaaatatgcatcttatccattacatacatgtatcatagtctaaggaaaggttctaagatagcattttatcttagaaccacttattatgttacggatggatgcatatacacagctctattccatattaatggtacatatatatgtaacatacacaagatagaaatttatggtgtcatatactcatacattgcatgtagccaatccctacacttcctagtggaaaagggcttagttgttgtacatatggtaacaaaagattgctgatttaataccaacatcccaagtcctaacacaacaaccacaaaaatatgaaatttattcatgtaagaaaacttacttaatgtaatacagtgttttcttatctttcccaaacttcgttatatagcaaaacccactaaatcgtaaatgaaaaatccatcttacctcccatgggcatataaagagaatggtatgtcagaaaacaagcatcgagatcttttaatgttggaccagttggtatcctgtaaattggatacctacaagcaaaacataagaacagatcagtcccttgtgtatctgtaagctgaatatgaaagcacataagtaaagaagaataccaggctacagatatccaacttgatggtagtatatcacaacttctaagggtcaccagttcagggaattgaaaagcaagatccattatctatcaaccacataaaagaaaatataatcaaacagcccaacaaagaaggaacaggaataacaaatgaccaaaatcccgtaggatagagtaagttagcacaccttatcagccaaaggctcacggctgtaaggagggtcccgttcaagatactcaaaaatcaagtaaccctgagaattgacagattctccatcatcactagaaaacccatctggtggaagggaatggtggtctctcaaagataatccgcccatccattgaggcacctcttctaacagatggggaagattcctcaatccccttccatgtacaggttcactatcactgctaccatcactacttgagtccctaaaatcactgtcactatcctcccccagttgcctatcaaagatccaaacaacaacattgtcatacaagtagctaactcttagcttaaggcattcaaccatagaagcagtaaaggtcatcattgtattgaggaaatcaaaatggcagaaaaccaaacatgaacaataatttcaatcctgcaacatacttaaaatccataaacacccgattaaaaatatgcaaagtatccactcaaacccgattgtttggcttgtatttggattttgcaccttttcaaacaatcactaaaatgaatcatctgtgcgggtccaaagaaagctatataccttgactttacagttggcttcacattctgggaataaatttggatcccagacagatacggaacatagtactgaacaacactatccttgtcattcagtacaagtggcactcctgcgccataagcactccattcccgaaaggattcccacaaatccccgagaacgaagtaaggctgaaactccgccccacacgccctgagtcctcttgtcgtcctctgcaagatccaagaaaactttcatgcttaacagttccacacatacacaatcataagaaaaatgagaaattcacaaaaaaataattatttttttctttttctgcacaaaaacattgcttcatttgtttcttttaattcacaccaaaaacatcctctccattccttccatggtcccttctataattaaacacggccataaatgttaaaccttcttcccattgaatgctttggtcacaactcacaaagacgtgaactttaaatcgaattccaataaacccaatttgaataaatgaaacacgatttcaccaacaacccagtttaataaaaaaaaaaaaaactttctttttcaaaacacttgttccagtgactcaaaagcaaaccttaggcagacactgagcaggcacggagggtgtgatcgcctgcaagaaccgctccagattactcaaccggttcgccaccggttcacacgaaggaaccgcgccactcttctttgcctcgtccgacccgacccggttctccggtatccggttacccgaatccaccgatttgtctctaacggagcgagacgcggcaacgtcgctttgggccctacgcagcttgtcattctccatagttagaagcgaccggcgagcctttgccggagaatagaaccgatcctcgccgcgagcgcgcccgaagttcaacccagtacccaacattcctatgcccaataaaaagagaagaaaaaaaaaaaggacccaggaataaaaagcggaactttaatggataaaacctcgagtgtgtgtggtcaacggtgtttgggatagaaaaaaaatggttgggaattggatgggagcgatgaggatctgtgtggggcgattctagggtttgggggaaaggggttgttgttgttgttgtgaggaagaagggtttgagtagagaggagttgtttctgtgaatcggagagaatgaaagaaaaaatagaaagggatgggtctttgagttaaatagaacaacgttacgatttctatattcctgcattttcaatactgtttttttttatttctcttggtgtcacatgatgctattattaattaattatatctttttatttatttattttttctctccttgaatgactgaataaattactattgttaattacgtgaatggcattttttttctgaataaagaagcgaacgtggagggagtcaggaaatggatctggtcgttggatgaggtggagtggaagggtaaggtagtcatttcaatcgagggctaagtggaacgcatggcgagaggcgaaaaacaattgaaaacgaaaaaataattaatgaatgaatctgactatgaccgaccatagactaaaactctttctttttctttgatttatttattgttgctatttttcattttttttactgcaatatttcttttttcttttttgataaaatttcttttcttttattgaaaaataggtatcacgcattttataaaatatttcaatttgatgcacacttttatcatctgttcagatgagaaaaaaataccggaaaaataacaaataaagacacatacatgtaaacaaattatacttgttaaaagacgttgtttcatgaattaacatatttacataagttatttttttacggcaactattcacataaaaaaatagttacataagttatgatttataaaataaaataaaatgccattgtaaaatttaattttataaagatagtatttaattttcataatagtaattgatgaataaaaaatattaaaattaaatatttttatttatttaaactttttttttttaaagttactttaatatttatggacgaaatggaataaaatcaaaaaatacttaacctaaaacttttttattttctttatatggagtggaaatcattaaatgtttttattgtattattttattcaatgactgtcatttaattttatgtgtgacaaattatgtttaaattttaattttttaataattgtattaaatgtctttattatattattttattcaacatctatcagttgtagaatcaaaggaataaattgaaaagatttataccaatgagttatgaacaatcacaaaagtgaatttggtgttatattttaacccaaaactttaatatttgagtttatagttttttttctcacttatatggagtttaactttttcatttttatcaaatgtgagacttcatctcatatttacactcaaataatttttttcctcaagtctgactctctttcacataacagtcttctcctttagcaaaagtcatttttaatcaataagtatccaatagtgatccttaaagcttaatcgttgagtgtcccgactagtctaactattaccactaacatgttgttagtatcttgtactgttgcaacacctacggaactcactgtctcgcattcatgcatggactcacccaccaataaaacttttgaaacaatggatcttcgtgtttatggattcaccgtcgacatcttactcgtttgagctggttactaagtcgaacctgactttgatatcaaatattgtacaattaagggggataaatcgaaaagatttatactaataagtcatgagtaactacataagtgaacttgacatcacactttaacccaaaatcgcaaggcttaggtttgtgaatttttttctcatttatatggtgttttatttttccattttgatcagatatgaaactccatctcatacttatactctaatattatcatttaattttatctaggccaatttgtgtttaaattttaatttattaaataattgtatttgataaataagtgtgttgatgcataaatatgtaatttggtgacaatttagtttttgaaattaaaaaaaaaaatctaatttaaattttgaatatgtaaaaagaacaacaattatatattgtcatataacttaatgatacattagtacatgaatttcaccatttaatattaaattgattaatgaatattatagtttaatggcaaaaataatctcacaatgaacgcatattttgtatactaaaaaactaggtcaaaacttatattctttcaggaattagattgttaataatttacacacgaacaaaaagaatcatttttaagcaaaaattgacatggataaaattaaatgataactattgaataaaataatacaataaacgcatttaatacagttattaaaaaaaaatcaaacacaatttggcatagataaaattaaatgatagcactactagaaatgtagccttttaatacaataaaggcatttaatgatttccacttcacataaagaaaataaaacaagttgtcggtaaatattttcattttattcactctcgttcataaatattaaagtaacttaaaaaaataaaggaaaatagttaatattaataaattgaagattaatttaaaattttagttctttcaaattgtcaatcatttatatatttagtgatcaagataataatatgttttttttattattattgaatcaaagatggtgggtgtctcttaaatctcaatcgacaaaatcaaagactaattttatctgtaatacatggcttgaaattaatctaagaaaattttatatgcataaaaaattaaacatgaattattctcttatatataaatagaaataaactttaaaatttgtcaaccttataagttaataatcttttttactttaattgtattggtacgaaaactaaaaagaaattctgaagtgtaaatgaaacctaaaaaaataatttatacacattcaatgagctgccaatataaattgatggattttttccacaaatatcatacgtagacctaattatctccccactttatacggaattaaccattttcaacacatttatttaaaaaaaaacattttcaacacatctaactcttctcacaaaaaaaaaaatggtaaaaaacacatttaactcttttttagtaatgatcttttctagacaaaacgataggaatgagatgaatcttcaatcttaggggtgggtacaacttaaaaaataatcaactagttttgaaaaaaaatcattttgatgcatctctatttaattatggcttcttatatttttatagcatattatcaatgtgtttagatttttttactaatttgaataatattattatttttatatatgaaaaagatattttaattttggaaaaaatagaaactaatttacactgaaaagtaggaggtcataattcgagagaattaattattaagttacctaataaattttaataaatacatagaagtttatttgagaaattactacgattatttatatttatttaattaaactataaaatcatttaacaaaatatagatgttcggtatgatggactaacatgattttacacttagttgtaaatgttagatttgactgataactttagaatttaaagattttggtcaacttcgatatccgggttgcacagttcgtgtcactttataaatcgtactcactgtatcacactttcctcccatatttggaatatgttaaaataaagttatctcactaatttataattttttttagatattattctaagtactaatatttagcttgaagtttataattctttatttttattattatatttattcctattttttaataattttatcatttatcatttcatttaatattaaaatattgctttaataaaataatgcattaaaaaaatggtggaaattgaactgcagctcgtgatcacggtaagatgtttatttgtgttggtttgtttggattttgaaaagacaaaaataggcttttggatgtgagggctgaggatggtgaaagtaaccgcgggaaaatgatgatgatggatgaaaagatccgaaattgacgacagctggtgatctgtgattggtggaaaccaaggcactcggattcgttatcagcacggtgacacgtgtgccagagagaaagtgtagtacacgtgtacggtccttatttaaggctgtaacactgttgaagctgtctttttcttatttatcctcctacatttttttaagaagaaaaaagtaaaagaaactcctctttaacatcatctgtaatttcggatcacgaaaataaacacgtgaacacacgaagtttttgttattttaaagtaaaaaaaaaacatttttatgcttttttatgacaatataaattctctattcacatcaatttaaaaataatttttaattatgtttaacagtttaaaactaaatttaacaaaatttaatataaattaatatgagatcacgtatcttttcatttttatttaaattgatgagacgctgagaaacgcagcatcatggcgcgtcgtaaagcgagaataaaagagttggggtcagagaatttttgaatcgggtcgctttgttttatttgaccggccaagttgaaataggttcgcacgcttagaggggtttatgatttgatgtgtttataatttggtttcttcggatttatttaattaaatgtttagtttatattttttaaaaaattcatttagttaaatgggttaaacttaagtctataaaaaacttgtcgatttatttatatatttaaatattattaaaattaatatatatatatacactattgtatttaataatcttatatcataaataaaataaatatttgaaatacaaaaatctttaaataggttaatagttcatatcaaatctaaaaagtctttgataagttaataggatagattagatcttaattttgtataacaaatcatatttatctatgtaaaatttgtcttaatctagtttattttcacccatatatagtattagaggttagcctatctttgtgtatgtagtaaataagaaaaataatgtttttagtagtgtaattttttttttattaaatttagttttgatctttatattttaaatccataaaactaattttgtatttataagatttttgtgtatgtgccaagagaatttacaaaaatgtgactagtgataatattttttcacagttaagagtgttttaaaacaaaaaataaaataaatataatttagaacgactgataataaaaaaactaaaaagttatttagaccctacatttatccttaattgtaaaaaaaagaaaaaaacaattgagcttcttttccattttaattattaaaaagggtgtgactgtgtaaggtaatatgaaatttgaaaatatgatcgtgatcttttatatatatttcgtatatatatatatatatattaaaatttgtgaataatttttttgttcgtatccatatgtcgatcaaataatagctcttgaggtttgtaagataggtatcgtaatgtgatactttgtaaagtttttgttcaattataaaccatatatattaattaatacattaatttgcaaaaatgaattacattgatcacctattaatcaatctcatatatattattttaatttatgaaaaattataacctaccaaaattaatttacttggatataattataacctaccgaaataaatagttaaatatattccatcaagctgcttattataacatatacgtaaattatgtaacaagtaatatttttaaaaaatatataattactatatttaattcattgatttttgtttttattcatgataattataattattatatttaattcattgattttattttaccatgcaagaaaaaaaataattacactaattatcaatcaatcacatatatattgttcgaatatacaaaaaattataacccactaaaattaagagttaaatatactttgtcaagttgtctgcaggagcatatgcgtaaattagacaatatgtaatattttaaaaaaattataattactatatttaattcattgatttttttattcatcataattataattagtatatttaattcgttgatttttttactgtacaaaaaaaaactcctttgaaatggtatataaatgattataaattaatcatttgaaatgctattaatcaaaacttaccatcaaaggtgcatcatttcttcgttttttttatcacaagtggctcctaaatatgatttaatgttagggattactctaaaaaaaaaaaattacgtcaccggtaaatgtgatacatttacaattttctccaatgcaaagagatttttgtatcaagctatatgtttcttttttcattttgattaacaaaacatgcatgatttattgacatatagcttggtccaaaaatctctcaccaactaaaaagttaggatcaaaattcttatgaaagacttcagtaaatgagtgtatgctaataatgttttgtggaataactggtttaggttatgtttgaaatgattaatattcttttcaaaacttaatttaaatggatgctttgtggtacggttttcctcaccatttgtatgcacacatcaaactttgatagcaggtaaatctcccttttcctaacttggaccgaaagatgttgataagttggtttcgatctacgacaccaactttattcccatgcatgacaagtttttaatttttttattcaaagtcaactttttatttttaaattaagagttgcatagtattattaaaaaaaaactagaacattaaatttacattgcttatcattttatttttattacaaataaattataaatatttcaaaaatgtaaattttgtctttttattttcttcaaaattctactttcaaatttactctaaaatgcttagagtatgtttagtttgcatttccattttctgtttttattttctattttcattttaaaaagattaggattctgaaaacatatttggtttgatttcttattttctatttttaggaaataaaaacactaaaaatttataatatgttgacttcttgtcatttcgtttttagtgttttcagtcgaaaacaagaatctcattttgggtaaaatgaaaatgagatgacaatgaatataattttaagcaatattttgaaaatgaaaagagttttcagaaaataaaaacagaaaatgaaaatgcaaaccaaacacacccttattgttttcattttttgattgtttttagtttttatttttactgaaaatgttttcaaaaatttaaccaaatacatttttatcaccattttctattttcggtaaaaatgaaaacagaaaacaagctaaccaaacaccccttaatttttgtattggggagagaggtgaggattggaagaggaatgatatcatccaataatttaattgaaagctatcacataataaatttattgaattttataaatttataataattatcttacaaattatgtatttaaatctgaacagatacataattattctagtttaactgatggaatcaaacctcccttgtgatgagatcaaatattatacttaaaacttgtgtctcatgcgtaaagtgttactaaaagaatttaacttaattgattaaataaaatatatattataaattcgttagtatcgtattaaaaaaaagttgaaaaacaaaatagttcacatcgaacatgagacacaagtttgaagtgcaatctttaatcccttcacagcggctgtatcatttcatttccctttcttttatttagttactctcttcagtcaagaggctaagcgctgtttaaatgttagtctgttttagatcaggcccacgatgactcaagctaaatacagtaagaatctccttaatccttcaaagcattcattttgatccacaaattacaattactgaacacaaaatcccgaacagtcactaaaacagaattatccctaaaaaaattatttatgtgaattattactataaaccctaatgatactcacaaattgccaaatcagatctaaacaatgatcaccatttctcttgtcacatgattgttaataaagaccaggcatttttggaaattaaaacaaaaaataatgcaacttcaaaagtattatgctttcttttactcaacactctactggacatctactggacataagacatgaggcacgagatcatcaaacgagaggtaataataggtaattcataaaaaaaaaaatttcacatacacattcttaatactttttataacactattttctttactattttatccagaacttctctttctctcttgtatctttctcaattctcataatacttttcttacaaatcataaaaaaagttgcatatacaatttctcgtaagatttaaatgcatttttttatattattgttcaattcaaatttatgtaataagactttgtattaaaagtcaacatagattaccatataaaattctactaattccgatctaagttacccattcttattcataaatttatgacaggggggttttgttatcaagattagcagaacaagtaggatatttctgaacttaaaaaccatttaatttttcttgctctctaatgtccaaataaaacaagtggtcagaagttagcaaatttggtcaaccttttcaacttgcctacacggggttccattcaaagataagaggcagagacaagttgtcaattccgtaggcggccataccctcacctcactctccgcatcttcttttgggtatatcatattctctcttcttcaatgttctctatccacaaccctccaaaatgaacatcttctttatcctcagatccctctcttctcccctgattctctcctacatcatcatcttctatctccttgccaaaaacacctcctgtggtgtggacccaaaatttcttgcatgtccacccacaacctgcgccaacaacaatcaaagtataagttatcccttttacatccaaggaaaacaagaacctttttgtgggaatcccggttttggcatctcttgtggcccaaatggttttccaatccttaatctgtctatacccaatacatcattcaccaaatatctacgaaaatcagacacttcgagtgtccaataccgcgttttcagtttcacgaccaaacaccaccaattccaaaggttgtcttcctcttcctcttactcagaatctcactcttcctagtacccgcgagttcgatattgctccgaatcaaacagacattagattgttctacggctgtgggtcattgccttggctggaagagcacaaagttgggtgctttaacgaaacgagttcagttctggcattgtataaagaggataaaaatataagttttgtgtcaaagaattgccagggcgaggttgtggatacgatagtggaagatggaataataggagggaatgaagaagcgttgacaaaagggtttttgctgacgtggaaggccggtaactgcagcgtgtgccacaacactggagggaggtgcggcttcgatttcgtcatgtacactttcaggtgcttctgcactgacagagttcattctgccaaatgtggtcctgatgatgatccaggttagttttttctaatgaccaaaacttctaataaagacaatgaccaggttattaaaatgtatggagacatgtaacgtttactcaaacttcataattgcaatggctgcatggttagatgctttgattggaaattactagtatatataaaggctgccagagaagacttaattaatttaaaactatttagtttatatggttgcaattggtgtgttttcttcgttttcagtttaaaaaaaagagtaaagtttaagactatacaatactataacagaaaactcaacagcaaagttctgttaacactaaacagaagtatcttcagtttgtttggcaaaagaaaggacggggattggtctaatcagtctcgaatgatggttatgtgagtgaaaaaaaaaaataggtgaggtagaataactaccaaacgaattagtcaaaccataatttccattttagcctgttaatactatgacttatgagatgaaggggtcggggaagcaaaaatctttttctatttcagtttttttaattttaattagaataataatattgtgaatgaataacaatactattataaatttaaattgatgttagcacaattttaaaataagaaagtcacacctatatcatttagttgacttcaaacgaattttgaatattccaaacaactgcttgttctttttctttttttcaatttcatgattgatacattatcaataatttaggtcgatgttcaattcctacttttcaatctaggatggatggctcttatgtctacgtttcaatgcatatattttgttatgggttatattactgagaactctaaagatagtaactaaaatatatttaatttacttcattaaatatttataattatttgaaaaaaatattaaatactttttttatgtttattgattgaggaaatattagatttatacatattagttttatacttttccaataatagtggaatatataaaatagttactggcatattgaaattaatgagatattagaaagataattaagttgaagagagaaattgatagaattagtgaattttaaataaggataattttaacattttttttaattaaaaacactattaactgatttctacaattttttaatatatgtaaattaataaaaataacttataataaaaaaatggaggaagtatttttcaatccttcaatatgaattctcgttgaaagaacacacatactaaagatagagatattctatcaccctctgagagagaaagaaatacactattaagtagtattaagtattagcaataatcttttttgttttcttatacttattttgtaaaatacatatattttttaaaatagaagaggatggaaaggaggaagcggggaaaagatgggtataattaataaaaataaaactgaaagttgaaaattaaattaacttagaagtgttttaatattttagttcttaaaactaaaaatgttatttttaaaaaaaattcaaaactactttgaacaaacatgtaattttttgtctttaaaatatatatttttaaaaataaacaaataagcttaatatattaaaagagtctaatttaattggttgaaaaaaatgcatgagtgtcatagagcctggtataatttctaattttttttattaattgtacttatttgttaattatcattgacagttgacagtaagcttttatttaaaaacttacaagatgcctaaatataataacaagttttaaactataattaaacagaaaaaatagttacgtaactatttttggaagtaataaaaaaagtttaaggcagtgaaccggactttctattgtaattcgttttctctttccgagaaaactatattaccgcaatagatattaagatgattaaacattttaacttttgattttgcatgaaaaaatataattgtatttgatactgacgttaaatgggaaaagagcatgtaatatagtttatttggcttgataaataactttagtggtatatcttaaaatcttaaattatttttttatttcacaatcccgcaaaacataaaatcaaagattttttaagattttttttatttttttctttattattagattttatttcttgagatcttagataacttaataagtcgtgtatgcatcatacttttaaatattttatttaaattttatattttttctcttcccctctctcattcttttttttattttttttattttcctttctctctcaagtttcactttcccttccccttctcttctttttattttcctttgttgctctagcatcactacatcagtatatgcgtcatattatatatatacatcagtgtatgcgtcacacacacacacacagatatatatatatatatatatatgttagtgcatattctggacaggacaaggagctaatttagtaaactcagcaaaaaaaaaaagagagagctaatttagactaacttaaactataattgttagttctctgaaaaagatattttcatattttggactttccctcttacctttctatggattacccaaatcccaattcttatgtcaaaatttctcacatttttttcatgtttactatttttagcaaaattcacaataatgggctgaagcctgaagttacttggttgggccactattaggctggactttgttactgccttttaagaatttcgttttttttccctaaaaatgtttagtcacattttttagcaaaatcttttcaaaatgaccttagtatgaatgaccaagttccttactaaatttaaaaacagctatccaacttcctgtagcaaaaaaaaaaaaaaaactatccaactacaagtttgcaacaattcaagtaacattttcacaacatctagaccaataatgctataaaaaaattcataaatatatatatacctgttgaattaagtctattgacttatataaaagattagatatcctagtttattccaaaattgatctactagtttattaaaagactttactaaaatcaaattttaaatagacttttaatttatgtttgagttatttctttttttaaagaagattagatcttaaaaaaagtcaatatcaattaaatagattacacttaatcctcatattaaaaaaataaattaaggctatcttgatctatttttttaaaacgtgactatatagtaaacgtccaaggtggtccataaaagaatacatggggttataaacatcaagtcggtccttcataatctaaaataagcatcaatttaatctagattaaattggagtttgtttaaatattgaaagacaaaataatgcctaaaatcttgaaggatcaacttaattaacatctcatataatcttaaaggatcaatttaaagtatctagtgactagtatgaatgtaatattcaataggatttattggaagtgttctgttaataaaatctcatgctaaaataagcttttctgtcagctattggtatagaagagaagttttttatattgaaaaaataggaagtttccacaaagtcttccagtctaaattagaatatagtctttgattgaaggtaacttattcaattatacaacaacaacaaagtgccttctcccgctaggagatagaaacttatttaattatattctatgatactcgaaccatattttataaaaacttgccgctccctcttcccaaccataaaggattttgcatactttttatccttgagtgcttttataagttttatttaattgaaatctaaatggttaaattcatagttaaatattcctattagcaatttttttttgttaagaagtatgatccatcaacccgttttactttctttttttttgtgtgtgattgcaacccgttttacttagtggcggaacagatctaacacacattttatcaaacactactcgttcacgtaattattctgtgaaggctatcacaacaaatcggcgatcaatattactttaattttcactaaaaaaaaaacattactttaatttttagaaaggagccctgcttcgaaattaggtctgaagttgaagaattcaatggtactataccttaattttttcccacggtctaatatgtttggatgtagggcatataatattgaaagtgacgtgtttcttatggtattttatatgctaccaatatggatgggggagatgatacttaccttgagtgctactattgttgagatctgaatccgttagatgaataaatgcttttttgagatgtgttaaagagtgagtatccaaattattctctgaaaataaatatttaaaagactttgattcttaaaaaacaaactatttagtctttcaaattataagtataagtatgagagacttcagttttagtctatacggagtataaataaatgaaccatattcataatcttgagaaattatttgctaactttttctgtaagatttaaattaagtatttactataaaagtatatttaagggttccataacaacacttactcgtggtactcacttattaattgttgatcgctgtagaaattgattccaactttaatcgaacttttatcattatgatttatgagttcaaatctaacatggtcactttaggcagagttttgtagataatatatacatataaatacgcaatggagtaatgccaattcttagtcgtaaggttgaacgggaataacttcctttgcctttaattatgaattcccaagttttcccatggaagactcaacatgaacctgttcctctacttaaactttcccggctccatttccctgatccttattttccagtctcttttcaattagagttaagctttcttcatcttgattttttaatagcacccttacattgaaaggttgctaatccaattctacttccttacaccgattctcttttggtcattttttgcttaaatatgaaacttatttttaaacaaaacccatgatttattgtcatcattaaagtttgagagactatattttattaaaaagttgctaaagaactaaaattgggcaagtattcgatttttttttttaaaagacaaaagattcaataacattaaaactgtccaggctacaagctgtaaccaagaccaaaacaaatacatagcaatatcagtcccatgcgattggacaaaatactaaacccataaaacaaagctaataatgaagcatgaatcagccataatacatgtataacccctgctaccaactaataaatgttgatacagaacttccacccttatttactgattactaataagtacattaatcactcatgattgacttccatctttgttatattatatggtacacaacctttgaggtttaaagtgcgtagagattaaattatgggatcgggtcataatttgatatggagaatcaaagttctggaacctcaaatatacagtgcgtagtgataagttatgagatgggatcgtaatccgatccggagggtacaatggtgaaattattatatagtttgatactttgatatcatcatattttcatagcaaaaaaatatcatcacattatcattgtatttgttttagtaaaaaacttaaattttctatatactattatttagaaaaaatgtagaaatcaatctagactataatattcatcggtcatataataatatttcctgttttatcacactcgagaagttgtttttaggcagacgtttagtttcacaagttcacacagagacagagagagtactagaaaactgagaaatgaggcccagaaatttgaagccactgattatgacactgaacgcgttttctatctggcctcgttgactggtcacgttgaagtgaagtgtgaacctaaccgcttaaaacgacatcaccattcgttaacgttgtctaattgccttattatttcccacactctttcaacgtcattcggtactctttttttctgcctctttttaagtatttcaacctcagaggcccactgcatttccaaactcttgtggactcgggacccttcccctgtgatatctttcttaaaaattcattactgtgcaaacaagtgtttttaaatctgatatgcgtctgatacacggtttaacaagcctcctagaccaagactagattcggatgcggaagggaagttttgaagcagagtcagttcagagattataaggagtgatcaaacaagtcaagtctttgtgcttgtttcaaaaggcgttagaaagaattggtctcactttctcttagggccagatatgtgtgtatttcttggtttcccactccctctttcatcttccgtagtcatcttctaattcaactgggaggagtataaaaacaagtcagacaaacttgagcttaatttacagttcagagaatcaaatcctagttgaattctgtttgcttttagatcatagacgtactgggtgactgaggaaggatgtgtggagtttatgaatttgtatcattgcttttgttttgtcaccttatgccacttctattggcggcggcttgtccacctctgctttcttgtggagatcttggcaatatcagtttcccctttactacaacagaacgccccgactgtggctttttacccatacggaattgtgaagatccactcaagttcaaaatgatccaattacagaataatggagaatggtttcgggttgtactcgtagctcagcttcggaacagttctatcataacttttcaaattagagacaaacatctctatgaccttctgcagaacgaaagttgtgaagctttcagatacaattatactattcctcccttctttcactttgctgctttacgtatccaataccacacaactctgttcaggtgcaaccgcagcctccatgtcagccctcccacgggcatgcttaattatacaaaatgccccgactacgatctctactacaagcacatcatcacggctgatgatgtgtctcggagttctttggtggcatgtacagaggtccagcttccaattaaagacgtgcctgacgctataaacccatttacctttgtaactgcagatatcatcattcgagtagacttaactgatgaatgtgcagattgcaactatcgccatggagggcagtgcaaacttgacagcacagagaaattttgttgtgccaatggtataaaacaacaaaaaccctgaaaacagaagcacccacgaatgcaatgcactaatccttgttgcttttatctgcttttcccattttaggttcttaaatgtttccttcacaatttgtgaaacattgtctacttttctgtaaatgtcccaagcccagttccttctcctacatcataaacagaataaaatattcaaaataggtcaagaaaagtgacattgatgatgtgcctaatctaagttctcttcttgtcttaaaatttcaaatgtacagtttactacacttagacggcaaggtctgagttgctctgattgattgtctaatgtctaatattattggtgcaacagtcacaattagcatgccatatttttctctgcttaactttatcgtcacgcttcaaatctgtgttatttgctaaattgtttcttctaattgcagcagttataaagaaagggttgagtttgaaggccaaactgggtataggtaatgtactgtttacataacttgatattccttttcaacatatgacttcaaaatcactgatcctcagtgcgaaagttcatttgtgtgttgaactcagagggtctgctgatttcttttcttttaattcccttatttccccctcaaaaaacatgagaacttatttaaaaaatttgtataactctatgtctctatcaatgctcattgctatctatcagaaaagtagcagcagtcacgcactctgttatttctcacggtatcttcaattgatattttctcttggcccgtttgcacaggtttaggtattggaatcccaagcatgttggcaattgggttgctgtttctctttctacaatacaaacgaaaatatggtacctcaggcggacaattggagtcaagagattcttattctgattcctcctcaaatcctcatggagaaagtagtagcgagtactttggagttccactcttcttgtacgagcagcttaaagaagcgacgaacaatttcgatcacaccaaagaacttggagacggaggcttcggtactgtctactatggtaggatacttaatcaaaccctacttgacacacaacattactctccttgtatggtttggaactactacatgctcattggtcctagtcaatctccaagtatccagggtccggaactactatgtgtctgtggtcctggtcaatctctataacacgcatagaagagtcttatcacttctttctcaaaattgaaaaaactcagttaaatatcatagagattggacagaacaatgaacatgtagagattaattggaatgatgaatgtgatagtcctgaactatataataatttcttgtcttatttttgctgtataactgagatatttaaattaaaacacagggaaactcccagatggacgtgaagttgccgtgaagcgcttatacgagcacaactggaagcgagtagaacagttcataaacgaagttaagatcctcacacgtttgcgtcacaaaaatcttgtgtcactctacggctgcacttcacggcacagccgtgaactcctacttgtgtatgaatacatttcaaacggcactgtagcgtgtcatctccatggtggattagcgaagcctggctccctaccatggtctacacgaatgaaaattgccgtagagactgctagtgcattggcttatctccacgcctctgacatcattcaccgtgacgtgaaaacaaacaatattctcctcgacaacaacttttgtgttaaggtagcagattttggactttcaagagacgtccccaacgatgtcacacatgtctccacagctccacaagggtccccaggttaccttgaccctgaatattacaattgctatcagcttactagtaagagtgatgtgtatagttttggggttgtgcttattgagctaatatcatccaagcccgctgttgatatgaacaggagcagggatgagattaacttgtcaaatctagccgtaaggaagattcaagaaagtgcagttagtgagttggttgatccttctcttggttttgattcagattgtagggttatggggatgatagtttcagtggcagggttggcttttcagtgtttgcaaagggaaaaggacttgagaccttctatgtatgaagtgttacatgaactgaggagaattgagagtgggaaggatgagggaaaggttcgagatgagggtgatgttgatggtgttgcagtttcacatagttgtgcacattcaccaccaccagcctcacctgagtgggaagaagttggattgttgaagaatataaagcctacttccccaaacactgtcactgataaatgggaaagtaaatgtactacgcctaatatcagtggttaatcatttagtctattattaattgttgataatccgtttttagtttttactatatcaactttcacctcattattagttgagtcacagtatattatcgtcgcattcgcttgtttaattgtgaagggtatgattaaattatacggcccattgtagtcaagcaaagagttgaagtgagggtatgacatgtgaatattagaaacaccggtacaaaaatacgaccaacaatttcaaacgaagttattgacatgaataagggttttcatttcctacttttttaagagaatctgacggtagagagcaagttgagtcagagtgtaagcaaactggtaaatgttaaaaatcgttgcattttgtaatcataaatgacgttttgtggtggcaattctttacgggcaaaaatggaatcattgttaactctaatcatttaacaatcacatttttgttagacaaaaatggttcaattgtgacatgtctgaaatgagagagcataaatcgacattttaaaaatgagaaactattacacaaatttttctatatttataataataaaaacatgtttaaattggtttgttacatgaattattggtacctttaagcacccttaatagatgggccaatatccttgtgactttgggtacattgattaacaccattaagcattacttgaattgtatggacatgaaggaaatcttaccatctttatctgttgagcttcaaatggttgtctgaccaaaatttccacctttaagggcaatgaaggtattcttgttaactcccgtcctgtccttgtggcaatatgctgtatcgtccataggagaatttgaatggaaaacacggtgggtcatgcatcactactgggataacgatttgtatctacctctttttttctccaagtctcccacgctcatttccctagtcaagttgttgttaagagagttcattacttgttcctcggaaaattgggaaattgggatggctccggttgatttgttctgcttccccttgttttctttccttctactcatcctgcgcgataccagaggcattctatcgtttgtgtctgctgagattgttcttgaagtagtattatctaatgattgttatgagtgctataatctccgtggcttgacaaggataaaacgttcaactgtactcaaggtacccttggcataaatttttcataagttacctcttattccatttgattgctacaattccttgttagttgttcttatagcgttagaccggattagtgagtttgctgactgctaacttggctgctttatgttaattactggcatcatgacaatagaatttgtttcactgtaacactttattgctctatagtctacctacaaaaattcttcaatttaactatatctgcagatgaacagcattatagagtaaacatgtcatgctgttacttctacttgtaatagtttttatgataaagaaaaaataaaagagatacaaaaaggaaataaaattaaatttataactatcaaattatgcaaattaattagtagttaatacacacaaatctaagagtgagaaaataacgaaacaatacaaataaaattatattttagaaaataaataatttttttaacaaaaaaaaaataattttaagaactttataaacacaacttttaaatcaagtttcaaagctgtgttttttttaaataaataaataaataaatctgatttcaatattaaatattaattgttaatataattacataaataaaaagagtagataatggttagtgggtggttacaacattatgtgattttagttttagttgagggggtaaaataataaaatgaacatataaactaaaatttgttcaaaattttgtatttcttatttgttcatgatttgtttacttttggtttaataatgaaaaggttgtttagtgtgtttttcaaaaataaaatgatctaccagaccaaatatcattatcaaaggtaatattctctgtctattgtaatatatattaaatattaatatttgatctttttcaagagagattaatacactaatcaagtatgcaatgctatacgcgactgcccgttataatcactatatatgatcataataataatttgcgcgtaatttttttttttttgaagcaatctgcaatcctaattccaaaaatagattataataaagaataataactattcgacatgaatgcggcctatggacatgaccttaactggccttacactatatatggtcctgtcttttgattaattttttcttcattcataaaatttaaatttgagaattcactaaaagtaattaattaattcttttttgtattaaaattagttataaataaaattggtaaacactatgcaccaataatataattaaacaaaaattataacacaaaaagagtattgattatgatgttaatacttgtgtagaacataatttgactttagattgggttggaggcgtaaaaaaaaaaaacattgttaatgaataggtaacaatgacaagagaccaatttaactttttggaattttcatctgattttaaaataatcatttatgtattttgatggttcaattgttgagttggtagccttagattatttttcattttgtaaggcctgagttttcatttaaagcaagatctatcaatgagagttattatgtttgaaggtaacataaaaaaaagatgcaaaagggttgaaaagtaaattcaatgtaaataagatcttattgaagaataataatgcctataaataagcattgacctttgtttacaagagaagatcctttgaatgtacattttaacaagtaatttttgaggaaatgggaatgaatggaaatggtttgtcccaagcatagttgaatacggtgacacctctgaagaggggttgaccattgctaacaagacaaacattaccggaaattttcaaaaattgagggtctattggttcaacagattgaaatcctttacagtttaacatcacattcgattggtaacatgaacaccaattcgcaatagtcacactccattctttctttccgtgtattgtagctcctgtttgtttttgacttatgtggatgtcgtgcaacgagcattggctgtagcctaaaaatatgatgcatgggataagttatgaacaacaatttatatgaaaaaaaaagtaacactaggagcaaattttacctttcgaaacgagggcaaggaataaaattatgttgagaatcttgatgattgaattagtcattttagataacacaaatgcaatgttcgattcttgaatttgtttaggcaaagaataacactatatgttgtgtattatttatacatatgagactttataaataaattacatgtgtatcaataatcaattggaaattttattttgtcaacaaaattggaaatttgatatttttttccatcattggagtagtaatatacaagataactatgtatctttgtgttgtgtgaagaaatttgagattttttttcatcattggagtaataatatacaagataactatgtatctttgtgttgtgtgaagaaatttgagatttttttccatcattggagcaataatatacaagataactatgtatctttgtgttgttttaaaattttgagatttttttccatgattggagcaataatatataagataactatgtatctttttgttgtgtggagaaatttgagattttttttcgatcattgtagcaataatatacaagataactatgtatctttgtattgtgtgaataatatcgcaaatataaaaggtcaaattatttaaaacgaagataaatggtagaaattttacaactataagatcaaattatttaagcttatatacatttttcatatatgtaatttaattttttttttttgtatttttttcttgtaaaatatgtttattttgattttcatctttaaagctctttagatagtgttttttcaccattcaaaatatttttttaaatgttcaaaacattatttaaagctctttaaagttgaaaaacaaaataaatacattttacaataactaaaacaaaaaaatgtaagataaaaaataaaaaaattctaaattataaagacaagaaatttatttaagccaattatttaaaagcttaaattagtttttttatgtaatttattatttttattcaatttgattctctatttttttattcaattcgatattctaatttttaaaaaaggttcgattttatttttgttgtccattttgtttgtgttcactaatagatagacgaggttaatgaaatggttctcctatacaatagccacatagaatccaaacttaagagtgttagacaaaataaacataaggactaaaaggatttaggattttttataaaaaaaatcaaatgatcaaattaaactaaaaaaatattaaaggttctaattcaaatcaaagtaataaatttgagaaaaatactaatcaacctatttatttaaaaccaagagagattgtagagattttgtcaatacaaattttttaatgactggttgctataatattgccttggagtaagaaaggtgttagagaaattgtagagatttgctcaatactacttttaatatttataactacaaaattaccttggagaaaaaaaggtgtttttaatctttaatcttttatgtaataaagaagaaattgcatagatttagtcaatacacatttttttattatttattgctacaacattgctttagagaaaaaatgtttttaatattttatataataaaaaagaaattgcatagaatgagtcaataccatatatttttcatgatttattctactatattgcctttgagaaaatatatatttttatatttttaaacaaaaaataaataaatttggttcaaattcctggtctcaaataagaaaaactttaagtagttgtttatttttttatttaaaattatgttaatgtctaaattcattgatatattaagtcaaatttttaattaaactagtcatcaaatcaatagatacttcacatcactaatatagtaagcttaaaaaaatatttacagtttattttataatatatatatatatatatattacatatgtaatatttaaaatatttataaatttatttaaaatataatgctgtaatgtaatattacttttgatttatatatataacagattagtttcaaagagaacatgtaatactcaagagaaaaatagaaaatgcaagaggacttattatctttttaatccttaaattttttaataaaattatttttagtcccttaactttttttcattcttatttttaagtctcttaaccctttttaacctttcttttaatctctaaatgaatggaaaaaaagtaagaaaaaaacaaattaaagactaaacataaagccaaccatttagagattaaaaacaaaaatcttaaaaaagtttatagaataaaaagataaataattaacccaaaacgaaatcatgtaatattttttttaaaaaaatattgagcttcactatggtacaaagttgtatatattttgtggctcttgggcataattatctctagagttagatttagttattttaaggggggaaaatcttaaaagatgatagagaatgaattccacgattgaataataataacaattatatagcaatgatgattgttgtttattgaaagcttgtgggtatgatacacaaaagaatagaagcacataattgataatattcatcttctaattttgccggatcaactagttaacttgaaattgaagagcgattaatatgtttcacccttcataacttataagtacatgttgataacaaaaaatagcatcttcaaatgacaaacacattaaatttattttaatcattttgaaaattatgtattttaaaaaatattgggcttcactatggtcacaaagttgtatagattttgtggctgttgggcataattatctctaaatttagattgttattttaaggaaaaaaaatcttgaaagatgatagagaatgaattcggtgattgaataataataacagttatataacaatgatgattgttgtttattgaaaaagcacataatattcatcttctaatttcatcggatcaactagttaacttgaaattaaggagcaattaatatgtttcatcgctcataacttataagtacaagttgataaccaaaaataatacctttaaataacaaacacattaaatttattttaatcattttgaaaattatgtattttaataaagaaaacaagacaatgtaataaaaagttaactactttaccatctccgttaacaacatacttttttttaatcacgtctcgttgaagatttttacataaaaataacaattgctagtctctcgtacaatacatgtacaggtgaaatataaatttataacgtaattaaaaaaatctccattaaacacgtatactttaattatagaagcttattttttgaaaactatcccttatatagagttatagtaagttagtgtttggacgtattgtgctgacaaaaaaaattaattgtcgatgaagacgaagacaggatgcggatgactaaaaaaacaaaacccaaaggcaaaggtaacccagtgaggagataaattattgtgcagaaaaaacgcatgacagctaatccaacaattatttggtaataaataaagttattaaatacattaataattaattgataatgatattcatatatttcattctgttatgactatttttttttttatttctctttgtcttttctattgcatcacatattatatatattattgatccgtttgttaaactttactaatattttttaaataaatatttttaatgtgttattttttctaaaaaaataattttttttaatttttaataaggaaattttatgttttttcaaaatcatttttttcgattaaaaaaaacaagatggacccattttttttgtttatttatgtattttactttcctttatctcttctctctccattacaatccaccctaaaaatggaagtggacacttataattttccttaattaatatgaaaataatttataaacacccaaatgtcaactgacgataatttttttaaaaaacttgattattgagttgtcaaataatttttttgtctaataaaaaataaaaatgaactaaaataatttattaaatataacctgaatgaatggatgagtaatatttttttataattactgtaaatagaatatttagtttcttaataaaatcctgacatatacttacaagtgttgactctttagataggagttatccgattaatcataagtcaaaagctttaataatacttaaagattatcatctctttcaaaaaatgtattaagtcaaacttaattttttcttacaaattcaaagttttgttagcatctgaattacattgatggatgaatatttattgtaaccttactaacagaccggttctaatggatttctcaaaataataatcacatttgatttaaaaaaattatttctacgataaattattttaaaaaatatatgaataataattttttattaaataactatgaggttggggtcggattttttccgtcaaaagtgaaatttgaaatcgaaatgaaatgattaatattcggcttgaactcatgccttgtccccggtaataattaatttataaaatattatatatatatatatatatatatatatatatatatatataacactaaaatata

SEQ ID NO: 28 - protein, 212 aa, glycine max
MPIRSRETAQRPGLLDRQRPLHAVLGGGKLADILLWKDKILSAAMVAGFSIIWFLFEVVEYNFLTLLCHILMAVMLILFVWYNAAGLITWNLPQIYDFQIPEPTFRFLFQKLNSFLRRFYDISTGKDLTLFFVTIACLWILSAIGNYFTTLNLLYIMFLCLVTLPIMYERYEYEVNYLASKGNQDVQRLFNTLDTKVLTKIPRGPVKEKKKK

SEQ ID NO: 29 - DNA, 1036 bp, glycine max
cctcctccatggaatgagtagttaattaaatttcttggttacaaagcttagaaccccaaatatcctttcgaacattacatatagttggcaccatgacgcatgaatcataacagtgctagaacttggttcaggagaaccaacaaattaacacaaagtacgaagataatttcaatcattcaaattggtaaccatgccaatccgttcccgtgaaactgcacagaggccaggattgttagaccgtcaaagaccactacatgcagtccttggcggaggaaagcttgctgatatattgctatggaaagacaagatattatcggcagcaatggtagcagggttctccatcatttggttcctctttgaagtggtcgaatacaattttcttactctactttgtcacatcctcatggccgttatgctcatcctattcgtatggtataatgcagctggacttatcacatggaacctgccacaaatctatgattttcaaatccccgaacccacctttagattcttgtttcaaaagctcaactcgttcttaaggagattttacgacatttcaactgggaaagacctcacactcttctttgtgacaattgcgtgtctctggatcttatcagctattgggaattattttaccactttgaatcttctatatatcatgttcctctgcctggtgactcttcccattatgtatgagagatatgaatatgaggtgaattatctagcaagcaaaggaaaccaagacgtgcagagattgttcaacacattggatactaaagttctaaccaagattccaaggggacctgtgaaagaaaagaagaagaaatgaagtttagatatgcaatataatgcacgaaataaagtacactataatataagctgtaccactaattggctacacttagatttagatctatctcctgatttaagtatgtaaaagaaaataatgcttgtaaatttatttctctgaaaaaacaagtgcttgcaacaacatatttctatgaaatatttagggtaatggaataattgaatgttc

SEQ ID NO: 30 - DNA, 8035 bp, glycine max
tgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagtccaggtcagattttatgtaggccaagtcgtagacctctgtaggccggcctagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttttttttttttttgatgatgggttgcttttaagtaattttacatcctctaattatttttttcttaaatatgggattaaattgattgttacttgttgaagctaaaaaaggtttataatgttatggactaaattgatgttgtattgatttattggttcaactaaaataagaatataatggtaacacaataataatatcatttactcgtaaattattcttggtataatttttaaaatgattattataaaaatcaacaaaattattatatatgatgagttataattagatgaggtatatattttacaccgtgaatgtttccttattttcttaaaaataaaatgatggtaaaccttaaatcctatagtagcgctaaactaggttaagcttgcaactcttattcgctaacctggtgacaacagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacataactttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgatctcatattttttctatggtcaataaaatattggctaaatgatacgtgtagtcttttatgttattgtttagatttaatttaattatttatcttttaaatttagtttcatttaatcattctgcccgtttaaaattaatgttgttaataattaacatatcga

SEQ ID NO: 31 - DNA, 11726 bp, glycine soja
tgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagcccaggtcagattttatgtaggccaagtcgtagacccctgtaggccggcttagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatgtctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagtgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagcccaggtcagattttatgtaggccaagtcgtagacccctgtaggccggcttagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatgtctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttt

SEQ ID NO: 32 - protein, 417 aa, glycine soja
MLICHHSLQDEISGPISARIFELCDPDFFPHTLQNSEVTSSSNCCHEEKSSYATTISPPLDVVDNNKFNINSNSSNIVTTTSSSTTTTSTTTNNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKTLADSRPRVRGRFAKNDDFGESHKQGSSNHEDDDEEIIVKEDDDMVDSSDIFAHISGVNSFKCNYSIQSLI

SEQ ID NO: 33 - DNA, 1332 bp, glycine max
atgttgcaggatgttgtccacccctcaacaccggctgagcaactccccattgatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctag

SEQ ID NO: 34 - DNA, 7714 bp, glycine max
tgtgtaaccaaagtctgttagtttaaaaaaaaattaattgtacccaaaaaataatataagtataaattggtacccaaaaaataatataagtatatatacatatataggtcggtctatcaggcttataaggctttctaataagcctaagtctggtttatttaatttaataggcttttaaaaaagtttgaacctaacattttaattaaataagtcagtccaggtcagattttatgtaggccaagtcgtagacctctgtaggccggcctagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggtcttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttataaatatttaggaacttgcttttaaggttttttttttttttgatgatgggttgcttttaagtaattttacatcctctaattatttttttcttaaatatgggattaaattgattgttacttgttgaagctaaaaaaggtttataatgttatggactaaattgatgttgtattgatttattggttcaactaaaataagaatataatggtaacacaataataatatcatttactcgtaaattattcttggtataatttttaaaatgattattataaaaatcaacaaaattattatatatgatgagttataattagatgaggtatatattttacaccgtgaatgtttccttattttcttaaaaataaaatgatggtaaaccttaaatcctatagtagcgctaaactaggttaagcttgcaactcttattcgctaacctggtgacaacagaactcttttgtttggacatttgcctagtaaagattagaagaggtccacaatggatggaaaggtacagttatacttctatttcggtaacttttagaatatttggcaaaattctcactaaacttgtagaatactttattcgttaaatagtacagttatctttttttttcaatgcaaaataatttaattgtcgaacataactttcaagagataaatgatttctacttacacggggaggataattgaatgtgggatttttttttattttacttctttagttctttatgggaaagaacttttaattaattcagaattcgatcataatttcgttaaagatcaaatatcaaatgattcaatcttaattttaatacattaattatttattataacgtgatttgatctcatattttttctatggtcaataaaatattggctaaatgatacgtgtagtcttttatgttattgtttagatttaatttaattatttatcttttaaatttagtttcatttaatcattctgcccgtttaaaattaatgttgttaataattaacatatcga

SEQ ID NO: 35 - DNA, 1251 bp, glycine max
atgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaactttggcggatagccggccccgggttagaggaaggtttgcaaagaatgatgactttggagagagccataaacaaggaagtagcaatcatgaagatgatgatgaagagataattgtgaaagaagatgatgatatggttgattcctcagatatctttgcacatatcagcggagtgaactctttcaaatgcaactattccatccagtccttgatttga

SEQ ID NO: 36 - protein, 443 aa, glycine max
MLQDVVHPSTPAEQLPIDEISGPISARIFELCDPDFFPHTLQNSEVTSSSNCCHEEKSSYATTISPPLDVVDNNKFNINSNSSNIVTTTSSSTTTTSTTTNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKIRERVGVAPIVEKMVENRLRWFGHVERRPVDSVVRRVDQMERRQTIRGRGRPKKTIREVIKKDLEINGLDRSMVLDRTLWRKLIHVADPT

SEQ ID NO: 37 - protein, 424 aa, glycine max
MLQDVVHPSTPAEQLPIDEISGPISARIFELCDPDFFPHTLQNSEVTSSSNCCHEEKSSYATTISPPLDVVDNNKFNINSNSSNIVTTTSSSTTTTSTTTNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKTLADSRPRVRGRFAKNDDFGESHKQGSSNHEDDDEEIIVKEDDDMVDSSDIFAHISGVNSFKCNYSIQSLI

SEQ ID NO: 38 - DNA, 7871 bp, glycine max
aaagaagtcagtggaagatttttcaaattgaaataaaaaaaatacgaatcttctactccttaaagaatttgggaaaaggggtagagtgattatacatgcatttgaaataaaaaaacacacacgccaaagagcccgttaatattggccacagggtggtgttgacgagttataactatgtgccttgaaggaaagaagtttttttttttaaaaaaaaaaaaaaaaaagccaaatggtattataaacaacaaacaaagcacaagtagtgcctaatacaagaaaagggatcaagaatgttacctgacccacatacgaaaatcatttagagcccacatacatcccatactatgatcaaaatagcgcatcatggaataaaaatgcaacagaacactatatgtaaaccatgctaaacttaaagctgctcccattgatgaaaataattgctctccacagctccataaaaatcttgtgtcacctacttaggccggaacacttcgagaaaaaccaaataccacgagcaagggctctacaaacattgccacggagaattacaccaacatgaaactggataaatataaccaggattccctttcagccattgccaagtgatcaggagtattacttgctctagcataccccatacatctatcctttttccatatgtttaaggtttgtgttaaaatacaatatttcaacttatatactcgtgtagcaaacaagctttcaatttctcaatcgtgttgagatgactcattgataatgaactttttctataaacgtaaatcataagatgctggtgttgttggttaattggtgttattgttgaccactttatattttcattattagattctaaacattttctctgtctatagggtttgctcttaactgattgttttgagttattaattattcaagttgatttgattggaattgtacatttggatctattatgcaatatatttagatttttttaaattatagagtttaatatttgttattcttcaatttagggtgaaatcttgcacaatgagaatgatgtagtctcctcaaaattttgaacataattgttttcaagtgataaatatgttgtctatgtgaagattgtcgatgtacttaatgatggacatattttatccaacaagttttgtaatgtcatcaatgaaaagcatgaatagtatttatatggagagaaatgcttataacatactctttttaacacatttttttttattggttaaaagttattaaaaattataaaaaaaaattaaatatgaaatggggtccacaaaattttttgtttttgataaatttcagttaataaaaaagaatgtccaaaaaaatgtactacaaaaattgtgttactaatatttattttcgtaagaatgcttctaaaaattaacattttttcaaaagttgtattaataaaattttaaatttctcaatttaaatataaaataagctaaattaaacccaactaattagtgaagaaagaaaacatttccataaattttcgatgtcgaaacaattgtaatgacttaaactcaaaacaaaacctatttttaaaccatgcaatttcttacacattataatcctacatgtgtatccatccatgcaccgcaacaaatgaagaaaaggaataaaaaaaaaggtgaatttgccaaaaacaaattaatggaatgctaacattaacataatgcgacataggacagcatatttaattgtgaattaaaaggcttggtcaacccatattcttctcatagttactagttactagttagccatgtcatttaattggcatatatataataatccatatataaatatacatgtcccaaaaattgaattccactacaccccaccaaagtagtgaattaatggccaaacgccactctcgcgttgacgcccgcgagtgccaggcttgacggcctacgtacaacctcatcaattcctctatatatgcacataaccaactttctctgtttttgacaccctcaaacaccccatcatcttatagctaacacaacacaacttctctctttttctctctcttcctaccactttaatttcgtttcatgtcaccttcttgttttcttttttagaaaaattaatccctttaaggcttaattttctaattaagcatgcaatattgtttttaattagtcaccttcaagtcgaggaacatacatatacacatatgtttgtgatcaccacaccaaattccacttctttctaagtgtgtgtatgtgtgtacatagattttgttttatatatatttgaggtttcaccttcattattgcctccttaattctgtgcaaaagaggattcaccaccaccatgttgcaggatgttatccacccctcaacaccggctgagcaactccccattgtaatcacctcaagcttgttacatttcatgtctcaatcatgtatgtttagtattaatgcatgtggtggaggaactaagatatatatatatatatatatatatatctttgtgtcatgcaatattttttaccttagctaaatggtttccttctgggttttgtgattgggtgtaacttcacaagtttgtttagatcaaggctttttttttctttcaaaataatgatttcacactcaaagagtgtataaaccttgggagggaagacaaagataggaaagagaccccagaaaaagaaaaaaaaaaaaaaagtctaagattgatgtaattaataatgtgtataaacctcgtaaggattaattgcaagcgtgtgtgaaaacccgttgagaagtaagaaatcacacctaagatgtgaaagttgtcaattaacttcttaataaacgtgaaaaaccacgtctgaaagtgaaatcaaacacagtctttctcatgaggcctgaaaattttgatggttttttctcctgggtgctctttcttatattttgtgaccaattattacaaatttctctaggaattaatcaattaattgatccagtcctgcattttaattctttccatgtatggatgatgaatatggttttgttaatgtttgttggcatgttaattagtttcatcctaaattagtctctgagaaacctgaacaatatttcccttatttggttatattcctggagctaagtggaagctgctaatgctattattcatcatagaaaacccacaagccatgctcacaatttgtgggacggaacgttttctttcctctatttatatggttggagagcataacgtaattttattgaccgaacaataggaaggaattaaagactccgtgagagtgaggatcaactctttttttatattcattatcatccaacggccctaccaccaaaatgcttaatgtcattttccaagggacaaagttgtaaaaatgatgtaacatttccgtgatagtaatttagacatagctttcggttcgtgtggaatatatatctctttgggggcttcttgtgtaagctttgctaattttttttatattctttattcagttagatagggagaaggaagggtctttgtgacaaacaattcacatggatagcatatatagcattattgggttcccttcaagtggctttcaattcccattgaaggacaattttttccacttaaaatcaacaaaaataacatagcatgaggttccttatttgtttttgcatacttgtatttcataataacattggctaccttggctcctacctgtcacatgaaggcatagatgcacatcactttcaccaataaattacccaacaatgcaaacccttttggaaaactgctctcaagtctattaccaaatatggacaaaactgactagactatgcaaccaaccacagtcatatgtagaattctttgttggggcctttgcacattgatttatatcgttttcttgttgataaagaaggaaggggtggtgttgtggcagatactataatatcatttggcatgtctttcacttttgaatgcccactacatctacaagccaaagcttcaattgattagagaatattagcttttgaatttcttttctgttctaagtgatcagatcagactcatgtctatcaacaaaaataggaattggattcagatttcaaacccatcataatgaagaaaataaaataaataaaaataaactagagagaatcacgtttcctagtttgtttttataaagaagaggctttgttaagactatttctcagactatgttttagtagtttatatggtaaacatgttgctcacaatgcttatatgtcaccactcactgcaggatgagatttcaagcccgattagtgctcgaattttcgaactttgcgagcctgatttcttcccagacacactgcaaaattcagatgttacttccagctcaaattgttgccatgaagagaagtcctcatatgccacaaccatatctccacctttagatttagtagacaacaagatcaatatcaataacaatagcaacatagtcactactacctcatctagcactaccacaaccagcaccacaaccaacaacaacaacaacaacacaacgaacagcaataacctgtccatcctctttgacactcaagatgaaattgacaatgacatctcagcctccatagacttctcatcatgtcgatctttagttgttccaccacttctctcaatctcaactcagcaggatcagtttgatttctcttcagctcagccacaggtgcaactatcagcagcagcaggttcagttttgaagggcctctctcactaccctacagatcatgtgattgcaccccttattggatctccgttaccatctgtttttgatgaagattgcatatcttccatcccttcttatgtgcctctcaacccatcatcaccctcttgctcttatctcagtcctggcataggagtgtacatgcctcctcctggttcccttaacactgccttatctgctgacagttctggattgtttggtgggaacattctactggggtctgaactgcaggcacatgaattggactatcagggagaaaatggtggaatattttgcacagattcaattcagagggtgtttaaccccccagatcttcaggtatgtgcaatttttcaagctaattagcatttaataggcatgtattgttagtgtaaatttttttacatattgtcaatcaattaaaaattattattgataaaacttttaaaataattattattaaaattaacgaactatcatacataacaattgtgattcagtactaatgtaaaaatctttacatgtcaatgagtatattttatttattctttttgtcaactgtcaagggactaaatgagaattttcaattccaatgttccatgtgttcagaaataaggaaaaagaggtacaatggtcaaagaagtttattaatgctgcaaatgttactataccttgcagcagtgaagtgtttttttataaattagaagaggctttatcagaggtggacttttgggggaaagctcagggtccacaaatctctaaactataaactcataggtgccccatgaccatcaaatagtaggtagcaaaagatatgagtccctttataaagtcaaatgcattaaaaaatactaaaatttggcctagcaagtaggaataaccactttcagccaaaagaaaaacagaaaaaaaggatcacaaacagtagcatcattagatagaaagacccacgtcaagggtggctgtgttatatctctttctaaagtctctataaagttaatgtgcagtttttaatagtgtgtgggccaacatctttccactttgtgttgaataagaagtaagaaatttatctttgattataatgtctctctcaggcacttggtactgagactcagaaacttgtagctggggctggaagttctgccactttgacaccagaaatctcacacttggaggactctaacttgaaagttggaaaactctctgttgagcagaggaaggaaaagattcatagatacatgaagaagagaaatgaaagaaatttcagcaagaaaatcaaggtactacatctgaacaccaacattaacaaacaaatttgaaatcttatattatgttatacatgatttccaatctattgcatcaatcaagccttgtgcatattttcaaaattcaactaatgatccaatgttttttaaaaaaaaaatgcagtatgcttgccgcaaaactttagcagatagccggccccgggttagaggaaggtttgcaaagaatgatgagtttggagagagccatagacaaggaagtagcaatcatgaagaagatgatgaagaagtaagattcccttaattggatacttttgttcaacttgccttagtctaaagttaaaatacaaaaaaattccttatcacttttaccttttcaattatttgatggcataattccatgatgctatatcccttccattttttgtacttgcagataattgtgaaagaagatgatgatatggttgattcctcagatatctttgcacatatcagtggagtgaactctttcaaatgcaactattccatccagtccttgatttgaattaaaattaactattagtttgactagtgaaagcttatctatataatcagcttctgtagattaattttggcagggcccttttcccatcccggttctctacaaatccgggtttagtggcttgaggaaactgaataattgaggtccaaataattataccaataagtgaagtgagttaggaacgtacagaaattagaaactgtgtacatttttgcagatatatattatctttttcattaagttgtaatcgaacatggagttgcgttaactaaggaaaattccagttgccccctcccaagattgatgtagcttctttttataaatatttaggaacttgcttttaagtagctttacatgctctaattattctttctacttaaatatgattgataaatattgaagctgagaattgttataatgttacgaattaaattaatagataacgtttataatgttacggactaaattgatgttgcgttgatttattggtttagtttaaggtggcttgaatctgatttgggtactcatattatatattatatagttaaaataagaatttaatagtaatgtagtaataatttaatgttttatattatcgtttaattataaattatcgttaacttttaaattgattaccataaaattaataaatttatttataatatataaaagttgaacttctcaacattaattatttaaaagaatgaaaaagaaacattttgtatcactatattaattaaaataacactcatgaatagtctttgatatattttagtaaaatcaataacttctcatattaatattaatttaaattatttgtaatatttaattttacgtgttgaattaatatgaatgaaaaaatattaaataaaataaatgtaaaagtttacactaatttaatattattatatatgcataaatttatttatttttataattaattactttaaaatatatttttggaaaaataataaattaaattcatgactttaaagttataaaataatatagtataatataaaaatataaaaattatatatatatatatatatatatatatatataaattaaattattaacatttatatttaatgttaaatatttaaattagaactaattagataaatgtaaattaatgatagaagactaatagttaatataaatataaggatttttatattatttttattgtaaattttattttataaactatttttaaaaaaactataaatgtaataattaaattataattattattggcttcagttttatataaaaatagctatatgtaaaaatatatgttacaaaatttatggtatgataaagttaataatatttttcaatttta

SEQ ID NO: 39 - DNA, 1272 bp, glycine max
atgttgcaggatgttatccacccctcaacaccggctgagcaactccccattgatgagatttcaagcccgattagtgctcgaattttcgaactttgcgagcctgatttcttcccagacacactgcaaaattcagatgttacttccagctcaaattgttgccatgaagagaagtcctcatatgccacaaccatatctccacctttagatttagtagacaacaagatcaatatcaataacaatagcaacatagtcactactacctcatctagcactaccacaaccagcaccacaaccaacaacaacaacaacaacacaacgaacagcaataacctgtccatcctctttgacactcaagatgaaattgacaatgacatctcagcctccatagacttctcatcatgtcgatctttagttgttccaccacttctctcaatctcaactcagcaggatcagtttgatttctcttcagctcagccacaggtgcaactatcagcagcagcaggttcagttttgaagggcctctctcactaccctacagatcatgtgattgcaccccttattggatctccgttaccatctgtttttgatgaagattgcatatcttccatcccttcttatgtgcctctcaacccatcatcaccctcttgctcttatctcagtcctggcataggagtgtacatgcctcctcctggttcccttaacactgccttatctgctgacagttctggattgtttggtgggaacattctactggggtctgaactgcaggcacatgaattggactatcagggagaaaatggtggaatattttgcacagattcaattcagagggtgtttaaccccccagatcttcaggcacttggtactgagactcagaaacttgtagctggggctggaagttctgccactttgacaccagaaatctcacacttggaggactctaacttgaaagttggaaaactctctgttgagcagaggaaggaaaagattcatagatacatgaagaagagaaatgaaagaaatttcagcaagaaaatcaagtatgcttgccgcaaaactttagcagatagccggccccgggttagaggaaggtttgcaaagaatgatgagtttggagagagccatagacaaggaagtagcaatcatgaagaagatgatgaagaaataattgtgaaagaagatgatgatatggttgattcctcagatatctttgcacatatcagtggagtgaactctttcaaatgcaactattccatccagtccttgatttga

SEQ ID NO: 40 - protein, 423 aa, glycine max
MLQDVIHPSTPAEQLPIDEISSPISARIFELCEPDFFPDTLQNSDVTSSSNCCHEEKSSYATTISPPLDLVDNKININNNSNIVTTTSSSTTTTSTTTNNNNNNTTNSNNLSILFDTQDEIDNDISASIDFSSCRSLVVPPLLSISTQQDQFDFSSAQPQVQLSAAAGSVLKGLSHYPTDHVIAPLIGSPLPSVFDEDCISSIPSYVPLNPSSPSCSYLSPGIGVYMPPPGSLNTALSADSSGLFGGNILLGSELQAHELDYQGENGGIFCTDSIQRVFNPPDLQALGTETQKLVAGAGSSATLTPEISHLEDSNLKVGKLSVEQRKEKIHRYMKKRNERNFSKKIKYACRKTLADSRPRVRGRFAKNDEFGESHRQGSSNHEEDDEEIIVKEDDDMVDSSDIFAHISGVNSFKCNYSIQSLI

(19) United States
(12) Patent Application Publication
(10) Pub. No.: US 2022/0030788 A1
(43) Pub. Date: Feb. 3, 2022

(54) GENOME EDITED FINE MAPPING AND CAUSAL GENE IDENTIFICATION

(71) Applicant: PIONEER HI-BRED INTERNATIONAL, INC., JOHNSTON, IA (US)

(72) Inventors: SABRINA HUMBERT, JOHNSTON, IA (US); MARK TIMOTHY JUNG, URBANDALE, IA (US); ZHAN-BIN LIU, CLIVE, IA (US); ROBERT B MEELEY, DES MOINES, IA (US); BO SHEN, JOHNSTON, IA (US); MARISSA SIMON, GRIMES, IA (US); PETRA J WOLTERS, KENNETT SQUARE, PA (US)

(73) Assignee: PIONEER HI-BRED INTERNATIONAL, INC., JOHNSTON, IA (US)

(21) Appl. No.: 17/277,131

(22) PCT Filed: Sep. 13, 2019
(86) PCT No.: PCT/US2019/051011

§ 371 Date: Mar. 17, 2021

Related U.S. Application Data
(60) Provisional application No. 62/746,259, filed on Oct. 16, 2018.
(60) Provisional application No. 62/753,609, filed on Oct. 31, 2018.

(51) Int. Cl. A01H 1/04; C12N 15/10

(52) U.S. Cl. CPC A01H 1/04; C12N 15/102

(57) ABSTRACT

The field is molecular biology, and more specifically, methods for editing the genome of a plant cell to identify causal alleles of a desired trait or to fine map a desired trait to small region of the genome for gene identification.

### FIELD

The field is molecular biology, and more specifically, methods for editing the genome of a plant cell to identify causal alleles of a desired trait or to fine map a desired trait to small region of the genome for gene identification.

### REFERENCE TO SEQUENCE LISTING SUBMITTED ELECTRONICALLY

The official copy of the sequence listing is submitted electronically via EFS-Web as an ASCII formatted sequence listing with a file named 7826 SeqList.txt created on Oct. 23, 2018 and having a size 154 kilobytes and is filed concurrently with the specification. The sequence listing contained in this ASCII formatted document is part of the specification and is herein incorporated by reference in its entirety.

### BACKGROUND

Genetic mapping in plants is the process of defining the linkage relationships of loci through the use of genetic markers, populations segregating for the markers, and standard genetic principles of recombination frequency. Fine mapping refers to the process of mapping of isolating a causal gene or sequence element responsible for a desired trait. This has usually been done by identifying recombination events using genetic markers in segregating plant material derived from parents differing in trait performance and sequence haplotypes at the region in question. First, a segregating population (F2, BC1, BC2 etc.) is created from parents differing in the trait of interest. This population is then genotyped with genetic markers polymorphic between the parents at regular, small intervals across the genome and phenotyped for the trait of interest. Genotypes at the markers are associated with the phenotypes to identify regions likely to control the trait of interest. Recombination events are then identified using existing markers in the identified genetic interval based parental alleles associated (or not) with the trait. New markers are often made in the smaller region to identify the most informative recombination events. Once events are identified, phenotypes are obtained from individuals with these events in order to further delimit the interval. This typically takes one or more iterations and leads to one or a small number of candidate genes or sequence motifs hypothesized to control the trait of interest. These are then tested with genome editing or transgenics.

However, not all genomic loci are susceptible to such methods. For example, some regions show low homology to a given line or population, or a non colinear region may prevent recombination from occurring. In such instances, there remains a need for a method to isolate a causal gene or sequence element responsible for a desired trait.

### SUMMARY

The methods described herein relate to generating novel genetic variants to accelerate existing genetic mapping procedures in genomic regions of low recombination or where presence-absence value (“PAV”) prevent recombination or when standard map based cloning methods are not optimal or may not produce the desired result. The methods described herein may also provide validation information for the targeted region and may be used to bypass the later stages of fine mapping altogether, thereby shortening the amount of time to validate a gene or region. Where phenotyping of a desired trait can be done in controlled environments, the methods described herein may reduce by a generation the time of creating the segregating population and genotyping to identify recombinants.

The present disclosure relates to methods for identifying a causal gene, genes, or genetic locus for a desired trait comprising 1) introducing a site-specific modification in at least one target site in an endogenous genomic locus in a plant or plant cell having a desired trait; 2) obtaining the plant or plant cell having a modified nucleotide sequence; 3) screening for the site-specific modification; and 4) screening for an increase or decrease in a phenotype of the desired trait. In a further embodiment, the method comprises identifying the causal gene or small region responsible for the desired trait.

The present disclosure also relates to methods for identifying a causal gene of a desired trait comprising 1) introducing at least one site-specific modification in an endogenous genomic locus in a plant; and 2) obtaining the plant having the site-specific modification; 3) screening the plant or the plant's progeny for the presence or absence of the desired trait, and 4) identifying the causal gene.

The present disclosure also relates to methods to create a novel haplotype in a genomic locus comprising 1) introducing at least one site-specific modification in an endogenous genomic locus in a first plant; 2) crossing the first plant with a second plant; 3) screening for the site-specific modification in the resulting progeny; and 4) correlating the haplotype of the progeny with its phenotype to establish a cause and effect relationship between the site-specific modification and the desired trait

The present disclosure also relates to methods for fine mapping a desired trait comprising 1) introducing a site-specific modification or deletion in at least one target site in an endogenous genomic locus in a plant; 2) obtaining the plant having a modified nucleotide sequence; 3) crossing the plant with a recurrent parent; and 4) screening for the loss or gain of a desired trait in the progeny of the cross. In one embodiment, the site-specific modification is a deletion.

In one embodiment, the methods further comprise introducing at least a second site-specific modification in the endogenous genomic locus, wherein said site-specific modification comprises at least one nucleic acid deletion, insertion, or polymorphism compared to the endogenous genomic sequence, allele, or genomic locus. In some embodiments, the methods further comprise selecting a plant having the modified nucleotide sequence. In some embodiments, the selected plant exhibits either an increased or decreased phenotype of a desired trait. A desired trait includes, but is not limited to, resistance to a disease, seed protein or oil concentration, grain yield, plant health, stature, stalk strength, and pest resistance.

In some embodiments, an endogenous genomic locus is located within a known QTL, is at least partially sequenced, or encompasses a random mutation fine-mapping. An endogenous locus may have low intrinsic recombination frequency, be a centromeric region, or comprise a non colinear region.

The methods disclosed herein may be used to create new haplotypes in a region by inserting genome edits, wherein the genome edited variants differ in key sequence motifs that may control the trait. An endogenous genomic locus may represent a unique haplotype that cannot be recombined with other haplotypes within the same interval. A unique haplotype may not be recombined with other haplotypes due to lack of homology.

In some embodiments, prior knowledge of the region of interest (genome sequence, marker trait associations, gene annotations, or quantitative trait loci (a “QTL”)) directs the design of the genome edits to target specific sequences, generating useful variants for testing. In another embodiment, the methods comprise deleting sequence regions to create specific variants, testing the specific variants for segregation of a desired trait, and identifying the causal gene or regions. In some embodiments, the identified region is smaller than the initial region of interest.

In one embodiment, the site-specific modification occurs in a non-coding region, a promoter, an intron, an untranslated region (“UTR”), or in a coding region. In some embodiments, the site-specific modification comprises a deletion, an insertion-deletion (an “INDEL”), or a single nucleotide polymorphism (a “SNP”) in the endogenous encoding sequence.

In some embodiments, the at least one site-specific modification comprises at least one double strand break introduced at one or multiple target sites. A double-strand break or site-specific modification may be induced by a nuclease such as but not limited to a TALEN, a meganuclease, a zinc finger nuclease, or a CRISPR-associated nuclease. A Cas9 endonuclease may be guided by at least one guide RNA. A guide RNA may direct a site-specific modification at a single or several specific target sites within the endogenous genomic locus.

### BRIEF DESCRIPTION OF THE DRAWINGS AND SEQUENCE LISTINGS

FIG. 1 shows fine mapping of causative gene by overlapping deletions over a 39 kb genomic deletion region.

FIG. 2 shows the protein and oil content of T1 seeds from deletion #1 and deletion #3.

FIG. 3 shows fine mapping of a soybean high protein QTL (qHP20) by overlapping deletion lines.

FIG. 4 shows a genomic sequence alignment of glyma.20g850100 from Williams 82 (SEQ ID NO: 30) and Glycine soja (SEQ ID NO: 31) and its paralogue glyma.10g134400 (SEQ ID NO: 38), including the 321 bp insertion from Williams 82.

FIG. 5 shows a protein sequence alignment of glyma.20g850100 from Williams 82 (SEQ ID NO: 36) and Glycine soja (SEQ ID NO: 32) and its paralogue glyma.10g134400 (SEQ ID NO: 40).

FIG. 6 shows a schematic of high protein and low protein alleles of glyma.20g850100.

FIG. 7 shows schematic of locations of Rcg1 and Rcg1b genes on an assembly of BAC sequences in the region of the non colinear fragment.

FIG. 8 shows the schematic of locations of the 26 genes in the ˜3.6 MB R Gene cluster on chromosome 10 in maize.

FIG. 9 shows an experimental scheme applied to a disease resistance locus. The recurrent parent in this case is susceptible to disease, and may be an elite breeding line. The genetic material generated during population development is resistant to disease, contains the resistance locus introgressed into the recurrent parent background at varying degree of purity depending on the breeding stage. This material may be a near isogenic line (NIL).

FIG. 10 shows editing and screening scheme for a dominant gain of function allele conferring disease resistance.

FIG. 11 shows multiple genomic alignments between a tropical line conferring resistance to anthracnose stalk rot and B73 displaying low homology in the region of interest.

FIG. 12 shows predicted gene models and expected deletions in region of interest conferring resistance to anthracnose stalk rot.

FIG. 13 shows an editing and screening scheme for a dominant gain of function allele conferring disease resistance with dual gene mode of action.

### DETAILED DESCRIPTION

It is to be understood that the terminology used herein is for the purpose of describing particular embodiments only, and is not intended to be limiting. As used in this specification and the appended claims, terms in the singular and the singular forms “a”, “an” and “the”, for example, include plural referents unless the content clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules. Unless defined otherwise, all technical and scientific terms used herein have the same meaning as commonly understood by one of ordinary skill in the art to which this disclosure belongs unless clearly indicated otherwise.

Methods are presented herein to edit a plant genome to fine map plants that have increased or decreased phenotype of a desired trait.

The methods disclosed herein may be used to fine map a causal gene, small genomic region, or chromosomal interval. Accurate identification of genomic sequence and gene models may increase the success of the methods disclosed herein because it allows for precise design of CRISPR-Cas guide RNAs targeting the genes or sequence regions thought to control the trait. In some embodiments, bioinformatic identification or other methods may be used to identify candidate causal genes in a chromosomal interval, then genomic edits are designed to delete the candidate genes, or portions thereof, sequentially in segments or regions, whereby a deletion or disruption of the causal gene produces either increased or decreased phenotype of a desired trait. Deletion of genes or portions thereof sequentially also can identify pairs of genes controlling the trait. The methods disclosed herein allow for dissection and identification of regions that have many genes with similar or duplicated segments. As provided herein, genes in a cluster may be sequentially deleted or deleted in pairs to determine the causal gene(s).

The term “allele” refers to one of two or more different nucleotide sequences that occur at a specific locus.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An allele is “associated with” a trait when it is part of or linked to a DNA sequence or allele that affects the expression of the trait. The presence of the allele is an indicator of how the trait will be expressed.

“Backcrossing” refers to the process whereby hybrid progeny are repeatedly crossed back to one of the parents. In a backcrossing scheme, the “donor” parent refers to the parental plant with the desired gene/genes, locus/loci, or specific phenotype to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F1 generation; the term “BC1” then refers to the second use of the recurrent parent, “BC2” refers to the third use of the recurrent parent, and so on.

As used herein, the term “causal gene” refers to any polynucleotide sequence encoding a gene that infers or contributes to a phenotype. In some embodiments, a causal gene infers or contributes to a desired trait. In some embodiments, a causal gene is located within a known QTL or a targeted genomic locus.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The phrase “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful in the embodiments disclosed herein when they demonstrate a significant probability of co-segregation (linkage) with a desired trait. Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

The term “crossed” or “cross” refers to a sexual cross and involved the fusion of two haploid gametes via pollination to produce diploid progeny (e.g., cells, seeds or plants). The term encompasses both the pollination of one plant by another and selfing (or self-pollination, e.g., when the pollen and ovule are from the same plant).

As used herein, the term “desired trait” refers a phenotype desired in a plant or crop. A desired trait may include, but is not limited to, disease resistance, an altered grain characteristic, grain yield, plant health, seed protein or oil concentration, pest resistance, abiotic or biotic stress resistance, drought tolerance, plant stature, or stalk strength.

A “favorable allele” is the allele at a particular locus that confers, or contributes to, an agronomically desirable phenotype, e.g., increased resistance to a disease in a plant, and that allows the identification of plants with that agronomically desirable phenotype. A favorable allele of a marker is a marker allele that segregates with the favorable phenotype.

A “genetic map” is a description of genetic linkage relationships among loci on one or more chromosomes (or linkage groups) within a given species, generally depicted in a diagrammatic or tabular form. For each genetic map, distances between loci are measured by how frequently their alleles appear together in a population (their recombination frequencies). Alleles can be detected using DNA or protein markers, or observable phenotypes. A genetic map is a product of the mapping population, types of markers used, and the polymorphic potential of each marker between different populations. Genetic distances between loci can differ from one genetic map to another. However, information can be correlated from one map to another using common markers. One of ordinary skill in the art can use common marker positions to identify positions of markers and other loci of interest on each individual genetic map. The order of loci should not change between maps, although frequently there are small changes in marker orders due to e.g. markers detecting alternate duplicate loci in different populations, differences in statistical approaches used to order the markers, novel mutation or laboratory error.

A “genetic map location” is a location on a genetic map relative to surrounding genetic markers on the same linkage group where a specified marker can be found within a given species.

“Genetic mapping” is the process of defining the linkage relationships of loci through the use of genetic markers, populations segregating for the markers, and standard genetic principles of recombination frequency. “Fine mapping” refers to the process of isolating the causal gene or sequence element responsible for a desired trait. This is usually done by identifying recombination events using genetic markers in segregating plant material derived from parents differing in trait performance and sequence haplotypes at the region in question. First, a segregating population (F2, BC1, BC2 etc.) is created from parents differing in the trait of interest. This population is then genotyped with genetic markers polymorphic between the parents at regular, small intervals across the genome and phenotyped for the trait of interest. Genotypes at the markers are associated with the phenotypes to identify regions likely to control the trait of interest. Recombination events are then identified using existing markers in the identified genetic interval based parental alleles associated (or not) with the trait. New markers are often identified in the smaller region that may aid in finding the most informative recombination events. Once events are identified, phenotypes are obtained from individuals with these events in order to further delimit the interval. This typically takes one or more iterations and leads to one or a small number of candidate genes or sequence motifs hypothesized to control the trait of interest. The candidate genes or sequences motifs may then tested with genome editing or transgenics.

“Genetic markers” are nucleic acids that are polymorphic in a population and where the alleles of which can be detected and distinguished by one or more analytic methods, e.g., RFLP, AFLP, isozyme, SNP, SSR, and the like. The term also refers to nucleic acid sequences complementary to the genomic sequences, such as nucleic acids used as probes. Markers corresponding to genetic polymorphisms between members of a population can be detected by methods known in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Methods are also known for the detection of expressed sequence tags (ESTs) and SSR markers derived from EST sequences and randomly amplified polymorphic DNA (RAPD).

“Genetic recombination frequency” is the frequency of a crossing over event (recombination) between two genetic loci. Recombination frequency can be observed by following the segregation of markers and/or traits following meiosis. A “low intrinsic recombination frequency” refers to a low number of recombination events identified based on the genetic map distance in a given region.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to alleles at a particular locus, or to alleles at multiple loci along a chromosomal segment.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

The term “hybrid” refers to the progeny obtained between the crossing of at least two genetically dissimilar parents.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “line” or “strain” is a group of individuals of identical parentage that are generally inbred to some degree and that are generally homozygous and homogeneous at most loci (isogenic or near isogenic). A “subline” refers to an inbred subset of descendants that are genetically distinct from other similarly inbred subsets descended from the same progenitor.

As used herein, the term “linkage” is used to describe the degree with which one marker locus is associated with another marker locus or some other locus. The linkage relationship between a molecular marker and a locus affecting a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units (or cM) of a single meiosis map (a genetic map based on a population that has undergone one round of meiosis, such as e.g. an F2). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “in proximity to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

Linkage disequilibrium is most commonly assessed using the measure r2, which is calculated using the formula described by Hill, W. G. and Robertson, A, Theor. Appl. Genet. 38:226-231 (1968). When r2=1, complete linkage disequilibrium exists between the two marker loci, meaning that the markers have not been separated by recombination and have the same allele frequency. The r2 value will be dependent on the population used. Values for r2 above ⅓ indicate sufficiently strong linkage disequilibrium to be useful for mapping (Ardlie et al., Nature Reviews Genetics 3:299-309 (2002)). Hence, alleles are in linkage disequilibrium when r2 values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “locus” is a position on a chromosome, e.g. where a nucleotide, gene, sequence, or marker is located. A locus may be endogenous to a plant in the plant genome (an “endogenous genomic locus”).

The “logarithm of odds (LOD) value” or “LOD score” (Risch, Science 255:803-804 (1992)) is used in genetic interval mapping to describe the degree of linkage between two marker loci. A LOD score of three between two markers indicates that linkage is 1000 times more likely than no linkage, while a LOD score of two indicates that linkage is 100 times more likely than no linkage. LOD scores greater than or equal to two may be used to detect linkage. LOD scores can also be used to show the strength of association between marker loci and quantitative traits in “quantitative trait loci” mapping. In this case, the LOD score's size is dependent on the closeness of the marker locus to the locus affecting the quantitative trait, as well as the size of the quantitative trait effect.

A “marker” is a means of finding a position on a genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA).

Depending on the DNA marker technology, the marker will consist of complementary primers flanking the locus and/or complementary probes that hybridize to polymorphic alleles at the locus. A DNA marker, or a genetic marker, can also be used to describe the gene, DNA sequence or nucleotide on the chromosome itself (rather than the components used to detect the gene or DNA sequence) and is often used when that DNA marker is associated with a particular trait in human genetics (e.g. a marker for breast cancer). The term marker locus is the locus (gene, sequence or nucleotide) that the marker detects.

Markers that detect genetic polymorphisms between members of a population are established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype.

Haplotypes tend to be more informative (detect a higher level of polymorphism) than SNP s.

A “marker allele”, alternatively an “allele of a marker locus”, can refer to one of a plurality of polymorphic nucleotide sequences found at a marker locus in a population.

“Marker assisted selection” (or MAS) is a process by which individual plants are selected based on marker genotypes.

A “marker haplotype” refers to a combination of alleles at a marker locus. A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non colinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”.

A “progeny plant” is a plant generated from a cross between two plants. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

A “recurrent parent” refers to the parent used for multiple backcrosses in a introgression scheme: the process of transferring a desired trait from a donor with an undesirable background to an elite with a more desirable genetic background.

A “reference sequence” or a “consensus sequence” is a defined sequence used as a basis for sequence comparison. The reference sequence for a PHM marker is obtained by sequencing a number of lines at the locus, aligning the nucleotide sequences in a sequence alignment program (e.g. Sequencher), and then obtaining the most common nucleotide sequence of the alignment.

Polymorphisms found among the individual sequences are annotated within the consensus sequence. A reference sequence is not usually an exact copy of any individual DNA sequence, but represents an amalgam of available sequences and is useful for designing primers and probes to polymorphisms within the sequence.

In “repulsion” phase linkage, the “favorable” allele at the locus of interest is physically linked with an “unfavorable” allele at the proximal marker locus, and the two “favorable” alleles are not inherited together (i.e., the two loci are “out of phase” with each other on different homologous chromosomes).

The embodiments disclosed herein may be used for any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the embodiments include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true first such as silver fir (Abies amabilis) and balsam fir (Abies balsamea); and cedars such as Western red cedar (Thuja plicata) and Alaska yellow-cedar (Chamaecyparis nootkatensis). Plants of the embodiments include crop plants (for example, corn, alfalfa, sunflower, Brassica, soybean, cotton, safflower, peanut, sorghum, wheat, millet, tobacco, etc.), such as corn and soybean plants.

Turf grasses include, but are not limited to: annual bluegrass (Poa annua); annual ryegrass (Lolium multiflorum); Canada bluegrass (Poa compressa); Chewing's fescue (Festuca rubra); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerata); perennial ryegrass (Lolium perenne); red fescue (Festuca rubra); redtop (Agrostis alba); rough bluegrass (Poa trivialis); sheep fescue (Festuca ovina); smooth bromegrass (Bromus inermis); tall fescue (Festuca arundinacea); timothy (Phleum pratense); velvet bentgrass (Agrostis canina); weeping alkaligrass (Puccinellia distans); western wheatgrass (Agropyron smithii); Bermuda grass (Cynodon spp.); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive, etc. Leguminous plants include beans and peas. Beans include guar, locust bean, fenugreek, soybean, garden beans, cowpea, mung bean, lima bean, fava bean, lentils, chickpea, etc.

### Genetic Mapping

It has been recognized for quite some time that specific genetic loci correlating with particular traits can be mapped in an organism's genome. The plant breeder can advantageously use molecular markers to identify desired individuals by detecting marker alleles that show a statistically significant probability of co-segregation with a desired phenotype, manifested as linkage disequilibrium. By identifying a molecular marker or clusters of molecular markers that co-segregate with a trait of interest, the breeder is able to rapidly select a desired phenotype by selecting for the proper molecular marker allele (a process called marker-assisted selection).

A variety of methods may be available for detecting molecular markers or clusters of molecular markers that co-segregate with a trait of interest. The basic idea underlying these methods is the detection of markers, for which alternative genotypes (or alleles) have significantly different average phenotypes. Thus, one makes a comparison among marker loci of the magnitude of difference among alternative genotypes (or alleles) or the level of significance of that difference. Trait genes are inferred to be located nearest the marker(s) that have the greatest associated genotypic difference. Two such methods used to detect trait loci of interest are: 1) Population-based association analysis and 2) Traditional linkage analysis.

In a population-based association analysis, lines are obtained from pre-existing populations with multiple founders, e.g. elite breeding lines. Population-based association analyses rely on linkage disequilibrium (LD) and the idea that in an unstructured population, only correlations between genes controlling a trait of interest and markers closely linked to those genes will remain after so many generations of random mating. In reality, most pre-existing populations have population substructure. Thus, the use of a structured association approach helps to control population structure by allocating individuals to populations using data obtained from markers randomly distributed across the genome, thereby minimizing disequilibrium due to population structure within the individual populations (also called subpopulations). The phenotypic values are compared to the genotypes (alleles) at each marker locus for each line in the subpopulation. A significant marker-trait association indicates the close proximity between the marker locus and one or more genetic loci that are involved in the expression of that trait.

The same principles underlie traditional linkage analysis; however, linkage disequilibrium is generated by creating a population from a small number of founders. The founders are selected to maximize the level of polymorphism within the constructed population, and polymorphic sites are assessed for their level of co-segregation with a given phenotype. A number of statistical methods have been used to identify significant marker-trait associations. One such method is an interval mapping approach (Lander and Botstein, Genetics 121:185-199 (1989), in which each of many positions along a genetic map (e.g., at 1 cM intervals) is tested for the likelihood that a gene controlling a trait of interest is located at that position. The genotype/phenotype data are used to calculate for each test position a LOD score (log of likelihood ratio). When the LOD score exceeds a threshold value, there is significant evidence for the location of a gene controlling the trait of interest at that position on the genetic map (which will fall between two particular marker loci).

### Markers and Linkage Relationships

A common measure of linkage is the frequency with which traits cosegregate. This can be expressed as a percentage of cosegregation (recombination frequency) or in centiMorgans (cM). The cM is a unit of measure of genetic recombination frequency. One cM is equal to a 1% chance that a trait at one genetic locus will be separated from a trait at another locus due to crossing over in a single generation (meaning the traits segregate together 99% of the time). Because chromosomal distance is approximately proportional to the frequency of crossing over events between traits, there is an approximate physical distance that correlates with recombination frequency.

Marker loci are themselves traits and can be assessed according to standard linkage analysis by tracking the marker loci during segregation. Thus, one cM is equal to a 1% chance that a marker locus will be separated from another locus, due to crossing over in a single generation.

The closer a marker is to a gene controlling a trait of interest, the more effective and advantageous that marker is as an indicator for the desired trait. Closely linked loci display an inter-locus cross-over frequency of about 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a target locus) display a recombination frequency of about 1% or less, e.g., about 0.75% or less, more preferably about 0.5% or less, or yet more preferably about 0.25% or less. Thus, the loci are about 10 cM, 9 cM, 8 cM, 7 cM, 6 cM, 5 cM, 4 cM, 3 cM, 2 cM, 1 cM, 0.75 cM, 0.5 cM or 0.25 cM or less apart. Put another way, two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are said to be “proximal to” each other.

Although particular marker alleles can co-segregate with increased or decreased phenotype of the desired trait, it is important to note that the marker locus is not necessarily responsible for the expression of the desired trait phenotype. For example, it is not a requirement that a marker polynucleotide sequence be part of a gene that is responsible for the phenotype (for example, is part of the gene open reading frame). The association between a specific marker allele and a trait is due to the original “coupling” linkage phase between the marker allele and the allele in the plant line from which the allele originated. Eventually, with repeated recombination, crossing over events between the marker and genetic locus can change this orientation. For this reason, the favorable marker allele may change depending on the linkage phase that exists within the parent having the favorable trait that is used to create segregating populations. This does not change the fact that the marker can be used to monitor segregation of the phenotype. It only changes which marker allele is considered favorable in a given segregating population.

### Marker Assisted Selection

Molecular markers can be used in a variety of plant breeding applications (e.g. see Staub et al. (1996) Hortscience 31: 729-741; Tanksley (1983) Plant Molecular Biology Reporter. 1: 3-8). One of the main areas of interest is to increase the efficiency of backcrossing and introgressing genes using marker-assisted selection. A molecular marker that demonstrates linkage with a locus affecting a desired phenotypic trait provides a useful tool for the selection of the trait in a plant population. This is particularly true where the phenotype is hard to assay. Since DNA marker assays are less laborious, cheaper, and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by marker assisted selection, it is not only the gene that is introduced but also the flanking regions (Gepts. (2002). Crop Sci; 42: 1780-1790). This is referred to as “linkage drag.” In the case where the donor plant is highly unrelated to the recipient plant, these flanking regions carry additional genes that may code for agronomically undesirable traits. This “linkage drag” may also result in reduced yield or other negative agronomic characteristics even after multiple cycles of backcrossing into the elite plant line. This is also sometimes referred to as “yield drag.” The size of the flanking region can be decreased by additional backcrossing, although this is not always successful, as breeders do not have control over the size of the region or the recombination breakpoints (Young et al. (1998) Genetics 120:579-585). The methods disclosed herein provide an alternative strategy to traditional mapping in cases of unsuccessful mapping due to low homology, low recombination frequency, or non colinearity. In classical breeding it is usually only by chance that recombinations are selected that contribute to a reduction in the size of the donor segment (Tanksley et al. (1989). Biotechnology 7: 257-264). Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will allow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers (See Tanksley et al., supra). When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

The key components to the implementation of marker assisted selection are: (i) Defining the population within which the marker-trait association will be determined, which can be a segregating population, or a random or structured population; (ii) monitoring the segregation or association of polymorphic markers relative to the trait, and determining linkage or association using statistical methods; (iii) defining a set of desirable markers based on the results of the statistical analysis, and (iv) the use and/or extrapolation of this information to the current set of breeding germplasm to enable marker-based selection decisions to be made. The markers described in this disclosure, as well as other marker types such as SSRs and FLPs, can be used in marker assisted selection protocols.

SSRs can be defined as relatively short runs of tandemly repeated DNA with lengths of 6 bp or less (Tautz (1989) Nucleic Acid Research 17: 6463-6471; Wang et al. (1994) Theoretical and Applied Genetics, 88:1-6). Polymorphisms arise due to variation in the number of repeat units, probably caused by slippage during DNA replication (Levinson and Gutman (1987) Mol Biol Evol 4: 203-221). The variation in repeat length may be detected by designing PCR primers to the conserved non-repetitive flanking regions (Weber and May (1989) Am J Hum Genet. 44:388-396). SSRs are highly suited to mapping and marker assisted selection as they are multi-allelic, codominant, reproducible and amenable to high throughput automation (Rafalski et al. (1996) Generating and using DNA markers in plants. In: Non-mammalian genomic analysis: a practical guide. Academic press. pp 75-135).

Various types of SSR markers can be generated, and SSR profiles can be obtained by gel electrophoresis of the amplification products. Scoring of marker genotype is based on the size of the amplified fragment. Various types of FLP markers can also be generated. Most commonly, amplification primers are used to generate fragment length polymorphisms. Such FLP markers are in many ways similar to SSR markers, except that the region amplified by the primers is not typically a highly repetitive region. Still, the amplified region, or amplicon, will have sufficient variability among germplasm, often due to insertions or deletions (“INDELs”), such that the fragments generated by the amplification primers can be distinguished among polymorphic individuals, and such indels are known to occur frequently in plants (Evans et al. PLos One (2013). 8 (11): e79192).

SNP markers detect single base pair nucleotide substitutions. Of all the molecular marker types, SNPs are the most abundant, thus having the potential to provide the highest genetic map resolution (PLos One (2013). 8 (11): e79192). SNPs can be assayed at an even higher level of throughput than SSRs, in a so-called ‘ultra-high-throughput’ fashion, as they do not require large amounts of DNA and automation of the assay may be straight-forward. SNPs also have the promise of being relatively low-cost systems. These three factors together make SNPs highly attractive for use in marker assisted selection. Several methods are available for SNP genotyping, including but not limited to, hybridization, primer extension, oligonucleotide ligation, nuclease cleavage, minisequencing and coded spheres. Such methods have been reviewed in: Gut (2001) Hum Mutat 17 pp. 475-492; Shi (2001) Clin Chem 47, pp. 164-172; Kwok (2000) Pharmacogenomics 1, pp. 95-100; and Bhattramakki and Rafalski (2001) Discovery and application of single nucleotide polymorphism markers in plants. In: R. J. Henry, Ed, Plant Genotyping: The DNA Fingerprinting of Plants, CABI Publishing, Wallingford. A wide range of commercially available technologies utilize these and other methods to interrogate SNPs including Masscode™ (Qiagen), INVADER®. (Third Wave Technologies) and Invader PLUS®, SNAPSHOT®. (Applied Biosystems), TAQMAN®. (Applied Biosystems) and BEADARRAYS®. (Illumina).

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes can be more informative than single SNPs and can be more descriptive of any particular genotype. For example, a single SNP may be allele ‘T’ for a specific line or variety with early maturity, but the allele ‘T’ might also occur in a plant breeding population being utilized for recurrent parents. In this case, a haplotype, e.g. a combination of alleles at linked SNP markers, may be more informative. Once a unique haplotype has been assigned to a donor chromosomal region, that haplotype can be used in that population or any subset thereof to determine whether an individual has a particular gene. See, for example, WO2003054229. Using automated high throughput marker detection platforms known to those of ordinary skill in the art makes this process highly efficient and effective.

In addition to SSR's, FLPs and SNPs, as described above, other types of molecular markers are also widely used, including but not limited to expressed sequence tags (ESTs), SSR markers derived from EST sequences, randomly amplified polymorphic DNA (RAPD), and other nucleic acid based markers.

Isozyme profiles and linked morphological characteristics can, in some cases, also be indirectly used as markers. Even though they do not directly detect DNA differences, they are often influenced by specific genetic differences. However, markers that detect DNA variation are far more numerous and polymorphic than isozyme or morphological markers (Tanksley (1983) Plant Molecular Biology Reporter 1:3-8).

Sequence alignments or contigs may also be used to find sequences upstream or downstream of the specific markers listed herein. These new sequences, close to the markers described herein, are then used to discover and develop functionally equivalent markers. For example, different physical and/or genetic maps are aligned to locate equivalent markers not described within this disclosure but that are within similar regions. These maps may be within a plant species, or even across other species that have been genetically or physically aligned with the plant, such as maize, rice, wheat, or barley. In some embodiments, the new sequences are modified or deleted by gene editing for fine mapping or causal gene identification.

In general, marker assisted selection uses polymorphic markers that have been identified as having a significant likelihood of co-segregation with a desired trait phenotype. Such markers are presumed to map near a gene or genes that provide the phenotype of a desired trait in a plant, and are considered indicators for the desired trait, or markers. Plants are tested for the presence of a desired allele in the marker, and plants containing a desired genotype at one or more loci are expected to transfer the desired genotype, along with a desired phenotype, to their progeny. Thus, plants with increased or decreased phenotype of the desired trait can be selected for by detecting one or more marker alleles, and in addition, progeny plants derived from those plants can also be selected. Hence, a plant containing a desired genotype in a given chromosomal region is obtained and then crossed to another plant. The progeny of such a cross would then be evaluated genotypically using one or more markers and the progeny plants with the same genotype in a given chromosomal region would then be selected.

### Gene Editing

Methods to modify or alter endogenous genomic DNA are known in the art. In some aspects, methods and compositions are provided for modifying naturally-occurring polynucleotides or integrated transgenic sequences, including regulatory elements, coding sequences, and non-coding sequences. These methods and compositions are also useful in targeting nucleic acids to pre-engineered target recognition sequences in the genome. Modification of polynucleotides may be accomplished, for example, by introducing single- or double-strand breaks (a “DSB”) into the DNA molecule.

Double-strand breaks induced by double-strand-break-inducing agents, such as endonucleases that cleave the phosphodiester bond within a polynucleotide chain, can result in the induction of DNA repair mechanisms, including the non-homologous end-joining pathway, and homologous recombination. Endonucleases include a range of different enzymes, including restriction endonucleases (see e.g. Roberts et al., (2003) Nucleic Acids Res 1:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.)), meganucleases (see e.g., WO 2009/114321; Gao et al. (2010) Plant Journal 1:176-187), TAL effector nucleases or TALENs (see e.g., US20110145940, Christian, M., T. Cermak, et al. 2010. Targeting DNA double-strand breaks with TAL effector nucleases. Genetics 186(2): 757-61 and Boch et al., (2009), Science 326(5959): 1509-12), zinc finger nucleases (see e.g. Kim, Y. G., J. Cha, et al. (1996). “Hybrid restriction enzymes: zinc finger fusions to FokI cleavage”), and CRISPR-Cas endonucleases (see e.g. WO2007/025097 application published Mar. 1, 2007).

Once a double-strand break is induced in the genome, cellular DNA repair mechanisms are activated to repair the break. There are two DNA repair pathways. One is termed nonhomologous end-joining (NHEJ) pathway (Bleuyard et al., (2006) DNA Repair 5:1-12) and the other is homology-directed repair (HDR). The structural integrity of chromosomes is typically preserved by NHEJ, but deletions, insertions, or other rearrangements (such as chromosomal translocations) are possible (Siebert and Puchta, 2002, Plant Cell 14:1121-31; Pacher et al., 2007, Genetics 175:21-9. The HDR pathway is another cellular mechanism to repair double-stranded DNA breaks, and includes homologous recombination (HR) and single-strand annealing (SSA) (Lieber. 2010 Annu. Rev. Biochem. 79:181-211).

In addition to the double-strand break inducing agents, site-specific base conversions can also be achieved to engineer one or more nucleotide changes to create one or more site-specific modifications described herein into the genome. These include for example, a site-specific base edit mediated by an C•G to T•A or an A•T to G•C base editing deaminase enzymes (Gaudelli et al., Programmable base editing of A•T to G•C in genomic DNA without DNA cleavage.” Nature (2017); Nishida et al. “Targeted nucleotide editing using hybrid prokaryotic and vertebrate adaptive immune systems.” Science 353 (6305) (2016); Komor et al. “Programmable editing of a target base in genomic DNA without double-stranded DNA cleavage.” Nature 533 (7603) (2016): 420-4. Site-specific modifications may also include a deletion of a nucleotide, or of more than one nucleotide.

In some embodiments, gene editing may be facilitated through the induction of a double-stranded break (a “DSB”) in a defined position in the genome near the desired alteration. In some embodiments, the introduction of a DSB can be combined with the introduction of a polynucleotide modification template.

A polynucleotide modification template may be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

A “modified nucleotide,” “edited nucleotide,” or “genome edit” or refers to a nucleotide sequence of interest that comprises at least one alteration when compared to its non-modified nucleotide sequence. Such alterations include, for example: (i) replacement of at least one nucleotide, (ii) a deletion of at least one nucleotide, (iii) an insertion of at least one nucleotide, or (iv) any combination of (i)-(iii). An “edited cell” or an “edited plant cell” refers to a cell containing at least one alteration in the genomic sequence when compared to a control cell or plant cell that does not include such alteration in the genomic sequence.

The term “polynucleotide modification template” or “modification template” as used herein refers to a polynucleotide that comprises at least one nucleotide modification when compared to the target nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSBs and modification templates generally comprises: providing to a host cell a DSB-inducing agent, or a nucleic acid encoding a DSB-inducing agent, that recognizes a target sequence in the chromosomal sequence, and wherein the DSB-inducing agent is able to induce a DSB in the genomic sequence; and providing at least one polynucleotide modification template comprising at least one nucleotide alteration when compared to the nucleotide sequence to be edited. The endonuclease may be provided to a cell by any method known in the art, for example, but not limited to transient introduction methods, transfection, microinjection, and/or topical application or indirectly via recombination constructs. The endonuclease may be provided as a protein or as a guided polynucleotide complex directly to a cell or indirectly via recombination constructs. The endonuclease may be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433.

As used herein, a “genomic region” refers to a segment of a chromosome in the genome of a cell. In one embodiment, a genomic region includes a segment of a chromosome in the genome of a cell that is present on either side of the target site or, alternatively, also comprises a portion of the target site. The genomic region may comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein, or complex of proteins, encoded by a Cas gene. A Cas endonuclease as disclosed herein, when in complex with a suitable polynucleotide component, is capable of recognizing, binding to, and optionally nicking or cleaving all or part of a specific DNA target sequence. A Cas endonuclease as described herein comprises one or more nuclease domains. Cas endonucleases of the disclosure includes those having a HNH or HNH-like nuclease domain and/or a RuvC or RuvC-like nuclease domain. A Cas endonuclease of the disclosure may include a Cas9 protein, a Cpf1 protein, a C2c1 protein, a C2c2 protein, a C2c3 protein, Cas3, Cas 5, Cas7, Cas8, Cas10, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component.

A guide polynucleotide/Cas endonuclease complex can cleave one or both strands of a DNA target sequence. A guide polynucleotide/Cas endonuclease complex that can cleave both strands of a DNA target sequence typically comprises a Cas protein that has all of its endonuclease domains in a functional state (e.g., wild type endonuclease domains or variants thereof retaining some or all activity in each endonuclease domain). Thus, a wild type Cas protein (e.g., a Cas9 protein disclosed herein), or a variant thereof retaining some or all activity in each endonuclease domain of the Cas protein, is a suitable example of a Cas endonuclease that can cleave both strands of a DNA target sequence. A Cas9 protein comprising functional RuvC and HNH nuclease domains is an example of a Cas protein that can cleave both strands of a DNA target sequence. A guide polynucleotide/Cas endonuclease complex that can cleave one strand of a DNA target sequence can be characterized herein as having nickase activity (e.g., partial cleaving capability). A Cas nickase typically comprises one functional endonuclease domain that allows the Cas to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are known.

A pair of Cas9 nickases may be used to increase the specificity of DNA targeting. In general, this can be done by providing two Cas9 nickases that, by virtue of being associated with RNA components with different guide sequences, target and nick nearby DNA sequences on opposite strands in the region for desired targeting. Such nearby cleavage of each DNA strand creates a double strand break (i.e., a DSB with single-stranded overhangs), which is then recognized as a substrate for non-homologous-end-joining, NHEJ (prone to imperfect repair leading to mutations) or homologous recombination, HR. Each nick in these embodiments can be at least about 5, 10, 15, 20, 30, 40, 50, 60, 70, 80, 90, or 100 (or any integer between 5 and 100) bases apart from each other, for example. One or two Cas9 nickase proteins herein can be used in a Cas9 nickase pair. For example, a Cas9 nickase with a mutant RuvC domain, but functioning HNH domain (i.e., Cas9 HNH+/RuvC−), could be used (e.g., Streptococcus pyogenes Cas9 HNH+/RuvC−). Each Cas9 nickase (e.g., Cas9 HNH+/RuvC−) would be directed to specific DNA sites nearby each other (up to 100 base pairs apart) by using suitable RNA components herein with guide RNA sequences targeting each nickase to each specific DNA site.

A Cas protein may be part of a fusion protein comprising one or more heterologous protein domains (e.g., 1, 2, 3, or more domains in addition to the Cas protein). Such a fusion protein may comprise any additional protein sequence, and optionally a linker sequence between any two domains, such as between Cas and a first heterologous domain. Examples of protein domains that may be fused to a Cas protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16. See PCT patent applications PCT/US16/32073, filed May 12, 2016 and PCT/US16/32028 filed May 12, 2016 (both applications incorporated herein by reference) for more examples of Cas proteins.

A guide polynucleotide/Cas endonuclease complex in certain embodiments may bind to a DNA target site sequence, but does not cleave any strand at the target site sequence. Such a complex may comprise a Cas protein in which all of its nuclease domains are mutant, dysfunctional. For example, a Cas9 protein herein that can bind to a DNA target site sequence, but does not cleave any strand at the target site sequence, may comprise both a mutant, dysfunctional RuvC domain and a mutant, dysfunctional HNH domain. A Cas protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein). In other aspects, an inactivated Cas protein may be fused with another protein having endonuclease activity, such as a Fok I endonuclease.

The Cas endonuclease gene herein may encode a Type II Cas9 endonuclease, such as but not limited to, Cas9 genes listed in SEQ ID NOs: 462, 474, 489, 494, 499, 505, and 518 of WO2007/025097, and incorporated herein by reference. In another embodiment, the Cas endonuclease gene is a microbe or optimized Cas9 endonuclease gene. The Cas endonuclease gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

Other Cas endonuclease systems have been described in PCT patent applications PCT/US16/32073, and PCT/US16/32028, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H—N—H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

A Cas protein herein such as a Cas9 can comprise a heterologous nuclear localization sequence (NLS). A heterologous NLS amino acid sequence herein may be of sufficient strength to drive accumulation of a Cas protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas amino acid sequence but such that it is exposed on the protein surface. An NLS may be operably linked to the N-terminus or C-terminus of a Cas protein herein, for example. Two or more NLS sequences can be linked to a Cas protein, for example, such as on both the N- and C-termini of a Cas protein. Non-limiting examples of suitable NLS sequences herein include those disclosed in U.S. Pat. No. 7,309,576, which is incorporated herein by reference.

The Cas endonuclease can comprise a modified form of the Cas9 polypeptide. The modified form of the Cas9 polypeptide can include an amino acid change (e.g., deletion, insertion, or substitution) that reduces the naturally-occurring nuclease activity of the Cas9 protein. For example, in some instances, the modified form of the Cas9 protein has less than 50%, less than 40%, less than 30%, less than 20%, less than 10%, less than 5%, or less than 1% of the nuclease activity of the corresponding wild-type Cas9 polypeptide (US patent application US20140068797 A1). In some cases, the modified form of the Cas9 polypeptide has no substantial nuclease activity and is referred to as catalytically “inactivated Cas9” or “deactivated cas9 (dCas9).” Catalytically inactivated Cas9 variants include Cas9 variants that contain mutations in the HNH and RuvC nuclease domains. These catalytically inactivated Cas9 variants are capable of interacting with sgRNA and binding to the target site in vivo but cannot cleave either strand of the target DNA.

A catalytically inactive Cas9 can be fused to a heterologous sequence (US patent application US20140068797 A1). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a FokI nuclease to generate double strand breaks (Guilinger et al. Nature Biotechnology, volume 32, number 6, June 2014).

The terms “functional fragment,” “fragment that is functionally equivalent,” and “functionally equivalent fragment” of a Cas endonuclease are used interchangeably herein, and refer to a portion or subsequence of the Cas endonuclease sequence of the present disclosure in which the ability to recognize, bind to, and optionally nick or cleave (introduce a single or double strand break in) the target site is retained.

The terms “functional variant,” “Variant that is functionally equivalent,” and “functionally equivalent variant” of a Cas endonuclease are used interchangeably herein, and refer to a variant of the Cas endonuclease of the present disclosure in which the ability to recognize, bind to, and optionally nick or cleave (introduce a single or double strand break in) the target site is retained. Fragments and variants can be obtained via methods such as site-directed mutagenesis and synthetic construction.

Any guided endonuclease can be used in the methods disclosed herein. Such endonucleases include, but are not limited to Cas9 and Cpf1 endonucleases. Many endonucleases have been described to date that can recognize specific PAM sequences (see for example—Jinek et al. (2012) Science 337 p 816-821, PCT patent applications PCT/US16/32073, and PCT/US16/32028 and Zetsche B et al. 2015. Cell 163, 1013) and cleave the target DNA at a specific positions. It is understood that based on the methods and embodiments described herein utilizing a guided Cas system one can now tailor these methods such that they can utilize any guided endonuclease system.

As used herein, the term “guide polynucleotide”, relates to a polynucleotide sequence that can form a complex with a Cas endonuclease and enables the Cas endonuclease to recognize, bind to, and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (See also U.S. Patent Application US 2015-0082478 A1, and US 2015-0059010 A1, both hereby incorporated in its entirety by reference).

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas endonuclease polypeptide. The crNucleotide and the tracrNucleotide of the duplex guide polynucleotide can be RNA, DNA, and/or RNA-DNA-combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the cRNA naturally occurring in Bacteria and Archaea. The size of the fragment of the cRNA naturally occurring in Bacteria and Archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 endonuclease complex is a duplexed RNA comprising a duplex crRNA-tracrRNA.

The tracrRNA (trans-activating CRISPR RNA) contains, in the 5′-to-3′ direction, (i) a sequence that anneals with the repeat region of CRISPR type II crRNA and (ii) a stem loop-containing portion (Deltcheva et al., Nature 471:602-607). The duplex guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) into the target site. (See also U.S. Patent Application US 20150082478 A1, published on Mar. 19, 2015 and US 20150059010 A1, both hereby incorporated in its entirety by reference.)

The single guide polynucleotide can form a complex with a Cas endonuclease, wherein said guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 20150082478 A1, and US 20150059010 A1, both hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 20150059010 A1, incorporated in its entirety by reference herein), or any combination thereof.

The terms “functional fragment”, “fragment that is functionally equivalent” and “functionally equivalent fragment” of a guide RNA, crRNA or tracrRNA are used interchangeably herein, and refer to a portion or subsequence of the guide RNA, crRNA or tracrRNA, respectively, of the present disclosure in which the ability to function as a guide RNA, crRNA or tracrRNA, respectively, is retained.

The terms “functional variant”, “Variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, and US 2015-0059010 A1, both hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence including, but not limited to, a nucleotide sequence within a chromosome, an episome, or any other DNA molecule in the genome (including chromosomal, choloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other Cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting may be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein may be performed in such a way that two or more DNA target sites are targeted in the method, for example. Such a method can optionally be characterized as a multiplex method. Two, three, four, five, six, seven, eight, nine, ten, or more target sites may be targeted at the same time in certain embodiments. A multiplex method is typically performed by a targeting method herein in which multiple different RNA components are provided, each designed to guide an guidepolynucleotide/Cas endonuclease complex to a unique DNA target site.

The terms “knock-out”, “gene knock-out” and “genetic knock-out” are used interchangeably herein. A knock-out as used herein represents a DNA sequence of a cell that has been rendered partially or completely inoperative by targeting with a Cas protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, and WO2015/026886 A1, both hereby incorporated in its entirety by reference.)

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (by HR, wherein a suitable donor DNA polynucleotide is also used). Examples of knock-ins include, but are not limited to, a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas endonuclease. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site of a Cas endonuclease. The donor DNA construct may further comprise a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. To determine the percent identity of two amino acid sequences or of two nucleic acid sequences, the sequences are aligned for optimal comparison purposes. The percent identity between the two sequences is a function of the number of identical positions shared by the sequences (e.g., percent identity of query sequence=number of identical positions between query and subject sequences/total number of positions of query sequence (e.g., overlapping positions)×100).

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. The frequency of homologous recombination is influenced by a number of factors.

Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and Maizels. (2014) PNAS (0027-8424), 111 (10), p. E924-E932).

Alteration of the genome of a plant cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., (Scientific American Books distributed by WH Freeman & Co.). Error-prone DNA repair mechanisms can produce mutations at double-strand break sites. The Non-Homologous-End-Joining (NHEJ) pathways are the most common repair mechanism to bring the broken ends together (Bleuyard et al., (2006) DNA Repair 5:1-12). The structural integrity of chromosomes is typically preserved by the repair, but deletions, insertions, or other rearrangements are possible. The two ends of one double-strand break are the most prevalent substrates of NHEJ (Kirik et al., (2000) EMBO J 19:5562-6), however if two different double-strand breaks occur, the free ends from different breaks can be ligated and result in chromosomal deletions (Siebert and Puchta, (2002) Plant Cell 14:1121-31), or chromosomal translocations between different chromosomes (Pacher et al., (2007) Genetics 175:21-9).

The donor DNA may be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas endonuclease and the target site, the donor DNA is inserted into the transformed plant's genome. (see guide language)

Further uses for guide RNA/Cas endonuclease systems have been described (See U.S. Patent Application US 2015-0082478 A1, WO2015/026886 A1, US 2015-0059010 A1, U.S. application 62/023,246, and U.S. application 62/036,652, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

### EXAMPLES

The following examples are offered to illustrate, but not to limit, the appended claims. It is understood that the examples and embodiments described herein are for illustrative purposes only and that persons skilled in the art will recognize various reagents or parameters that can be altered without departing from the embodiments disclosed herein.

### Example 1. Fine Mapping of Causative Gene in High Protein Mutants from Fast Neutron Mutagenesis in Soybean

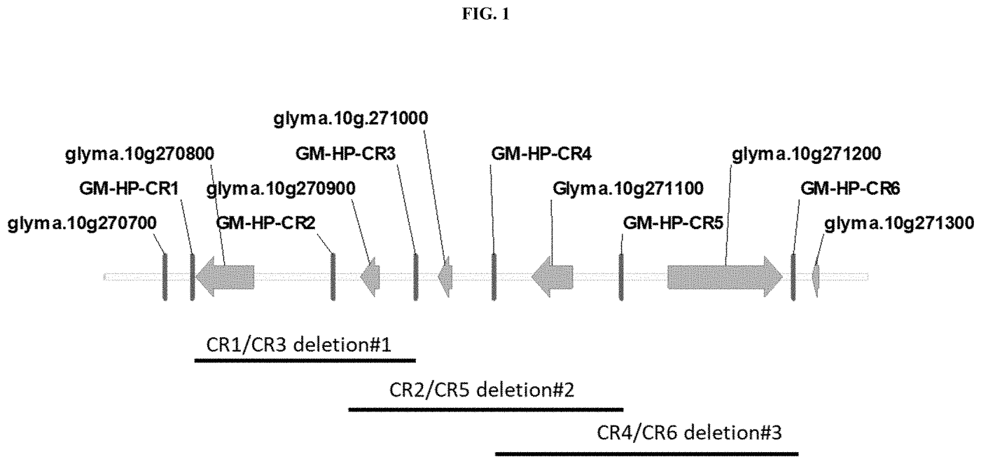

Protein is the most valuable component in soybean seed. One high protein/low oil mutant line (PO1) was identified from a fast neutron mutant population (Bolon et al. 2011 Phenotypic and genomic analysis of a fast neutron mutant population resource in soybean. Plant Physiol 156:240-253). The P01 mutant was mapped to a 39 Kb deletion on chromosome 10 which contains three possible candidate genes. The causative gene, however, was not identified due to no recombination in deletion region. CRISPR/CAS9 was used to create three overlapping deletions in this region to identify the causative gene responsible for high protein/low oil content (FIG. 1).

Six guide RNAs (gRNAs) targeting specific sites in the region of interests were designed as shown in Table 1. The genomic sequence of this region is shown in SEQ ID NO: 27. Each pair of gRNAs and CAS9 were delivered to soybean by transformation. T0 plants with heterozygous CR1/CR3 deletion #1 and CR4/CR6 deletion #3 were identified based on molecular analysis of variants. T1 seeds from selfed T0 plants were segregating for 1:2:1 of homozygous deletion, heterozygous deletion and wild type.

TABLE 1
guide RNA designed to produce deletions in region of interest

| | Approx- | | | | |
|---|---|---|---|---|---|
| Edit | imate | | Guide | | Guide |
| design- | expected | | 1 | | 2 |
| ation | deletion | | SEQ | | SEQ |
| (guide | size | Guide 1 | ID | Guide 2 | ID |
| pair) | (bp) | name | NO: | name | NO: |
| GM-HP- | 20,118 | GM-HP-CR1 | 11 | GM-HP-CR3 | 13 |
| CR1/CR3 | | | | | |
| GM-HP- | 25,988 | GM-HP-CR2 | 12 | GM-HP-CR5 | 15 |
| CR2/CR5 | | | | | |
| GM-HP- | 26,957 | GM-HP-CR4 | 14 | GM-HP-CR6 | 16 |
| CR4/CR6 | | | | | |
| | | GM-RET- | 17 | | |
| | | CR1 | | | |

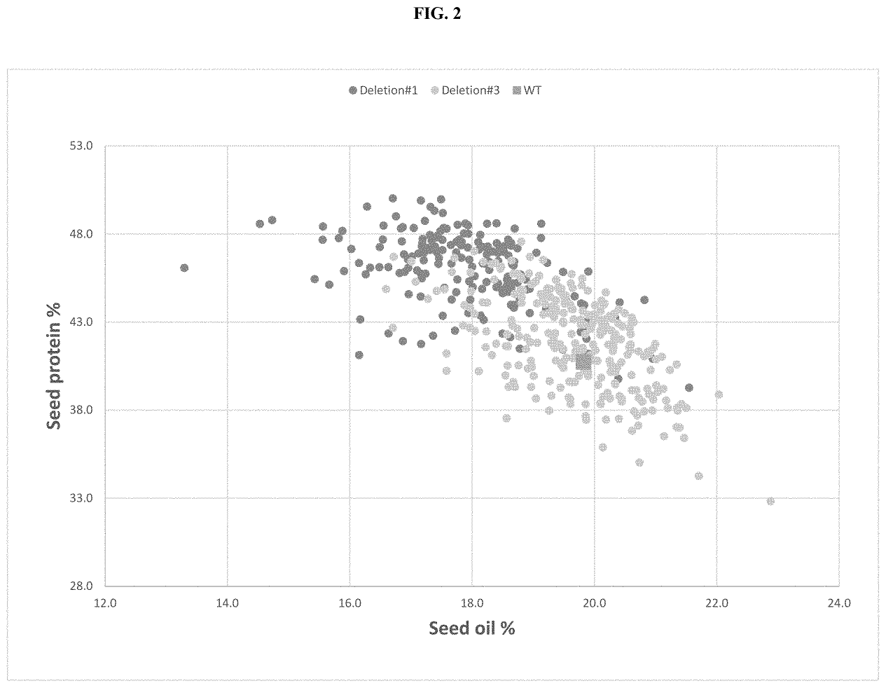

T1 seeds protein and oil content were determined by the single seed NIR as described previously (Roesler et al. 2016, Plant Physiol. 171(2):878-93). T1 seeds from CR1/CR3 deletion #1 line showed an increase in protein content and a decrease in oil content as compared to T1 seeds from CR4/CR6 deletion #3 line and wild type average, indicating that the deleted fragment in CR1/CR3 deletion #1 line contains causative gene for high protein/low oil (FIG. 2). Sequence analysis of the deletion #1 region identified two potential genes, Glyma.10g270800 and Glyma.10g270900. Because the Glyma.10g270800 gene was not deleted in the original fast neutron P01 mutant, the second Glyma.10270900 was most likely the causative gene for high protein content. Glyma.10g 270800 encodes a reticulon-like protein which may play an important role in regulating oil and protein biosynthesis in endoplasmic reticulum. To validate that glyma.10g270900 is the causative gene for high protein phenotype, a guide RNA (GM-RET-CR1, SEQ ID NO: 17 in Table1) was designed in the exon1 of the Glyma.10g270800 to knockout out the reticulon-like protein. If the reticulon-like knockout line shows high protein phenotype, this would validate that reticulon-like protein is involved in regulating protein and oil content in soybean seed. Knockout of reticulon-like gene in elite soybean by CRISPR/cas9 is expected increased seed protein content.

### Example 2. Fine Mapping of a Soybean High Protein QTL (qHP20)

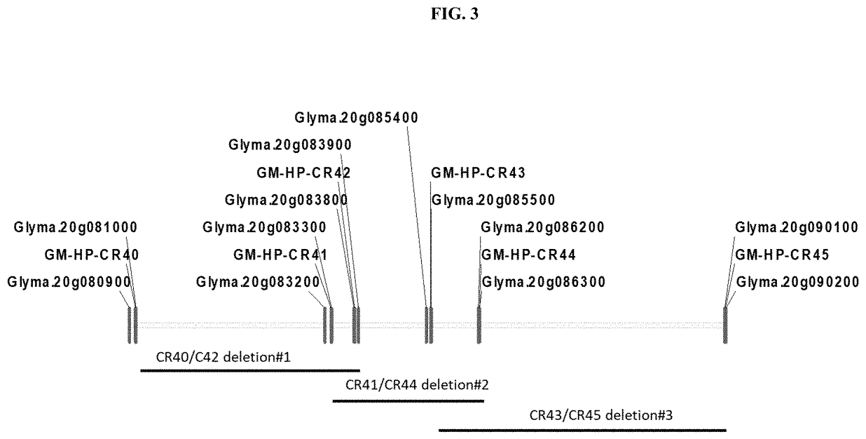

Given the importance of protein content in soybean, the quantitative trait loci (QTL) associated with high protein content have been mapped intensively. One major high protein QTL on chromosome 20 (qHP20) was detected by multiple mapping studies and showed consistent effects on seed protein and oil content (Chung et al 2003 Crop Sci 43:1053-1067; Nichols et al 2006 Crop Sci 46:834-839; Bolon et al. 2010 BMC Plant Biology 10:41; Hwang et al 2014 BMC genomics 15:1). The qHP20 was mapped to a 2.4 Mb interval and cannot be advanced further because of low recombination rate in the region. Using CRISPR/cas9 technology, a series of overlapping deletion lines are created to fine map the qHP20. The guide RNA pairs targeting specific sites within the qHP20 region are designed to create overlapping dropouts in the qHP20 QTL region. When delivered to the high protein donor line in combination with Cas9, these guides are expected to produce genomic deletions ranging from approximately 700 kb to 1.4 Mbp (Table 2). T0 plants with deletion are selected and genotyped to verify the occurrence of the expected deletion. T0 plants may be edited on a single or both chromosomes, thus respectively hemizygous or homozygous at the edited locus. Phenotype analyses, such as protein and oil content in seeds are performed at the T1 seeds to identify the sub-region of interest that can change seed protein content. By the same mapping techniques as traditional QTL mapping using near isogeneic lines, the QTL can be mapped by overlapping deletion lines created by CRISPR/Cas9. Table 4 lists possible protein phenotypes of deletion lines and the position of QTL. For example, if both CR40/CR42 and CR41/Cr44 deletion lines show reduced protein content while CR43/CR45 deletion line shows no protein change, the qHP20 will be defined to an interval between CR41 and CR42 (See FIG. 3). An additional round of guide RNAs may be designed to further narrow down the candidate genes in the sub-region if needed. After a candidate gene is identified, the function of the gene can be confirmed by additional editing experiments such as frame-shit knockout or precise segment dropout/replacement (See Table 3).

TABLE 2
guide RNA designed to produce deletions in qHP20 region

| | Approx- | | | | |
|---|---|---|---|---|---|
| | imate | | Guide | | Guide |
| | expected | | 1 | | 2 |
| Edit | deletion | | SEQ | | SEQ |
| designation | size | Guide 1 | ID | Guide 2 | ID |
| (guide pair) | (bp) | name | NO: | name | NO: |
| GM-HP- | 1,041,115 | GM-HP-CR40 | 18 | GM-HP-CR42 | 20 |
| CR40 + 42 | | | | | |
| GM-HP- | 706,332 | GM-HP-CR41 | 19 | GM-HP-CR44 | 22 |
| CR41 + 44 | | | | | |
| GM-HP- | 1,401,600 | GM-HP-CR43 | 21 | GM-HP-CR45 | 23 |
| CR43 + 45 | | | | | |
| | | GM-CCT-CR1 | 24 | | |
| GM-CCT- | 321 | GM-CCT-CR2 | 25 | GM-CCT-CR3 | 26 |
| CR2 + 3 | | | | | |

TABLE 3
Expected results for gene edited fine mapping of qHP20 based
on protein phenotype of the overlapping deletion lines

| | CR40/ | CR41/ | CR43/ | |
|---|---|---|---|---|
| | CR42 | CR44 | CR45 | Location |
| | deletion | deletion | deletion | of qHP20 |
| Seed protein | reduced | no change | no change | between CR40 |
| content | | | | and CR41 |
| Seed protein | reduced | reduced | no change | between CR41 |
| content | | | | and CR42 |
| Seed protein | no change | reduced | no change | between CR42 |
| content | | | | and CR43 |
| Seed protein | no change | reduced | reduced | between CR43 |
| content | | | | and CR44 |
| Seed protein | no change | no change | reduced | between CR44 |
| content | | | | and CR45 |

### Example 3. Validation of qHP20 QTL by Genome Editing

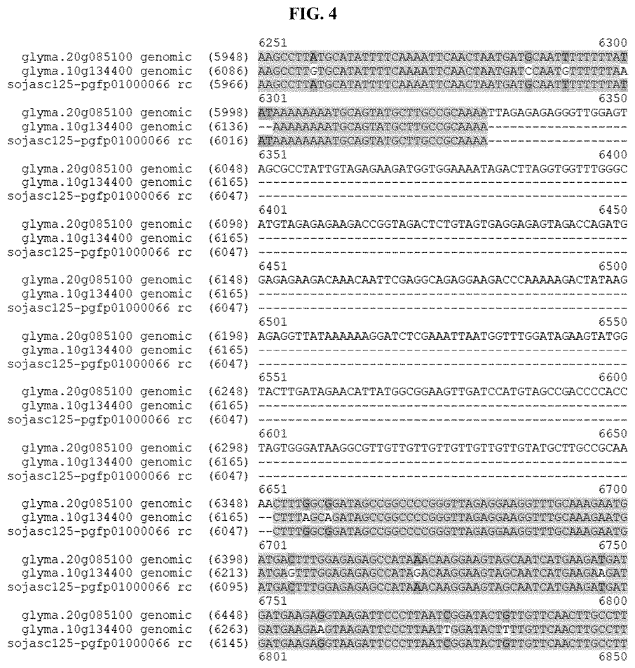

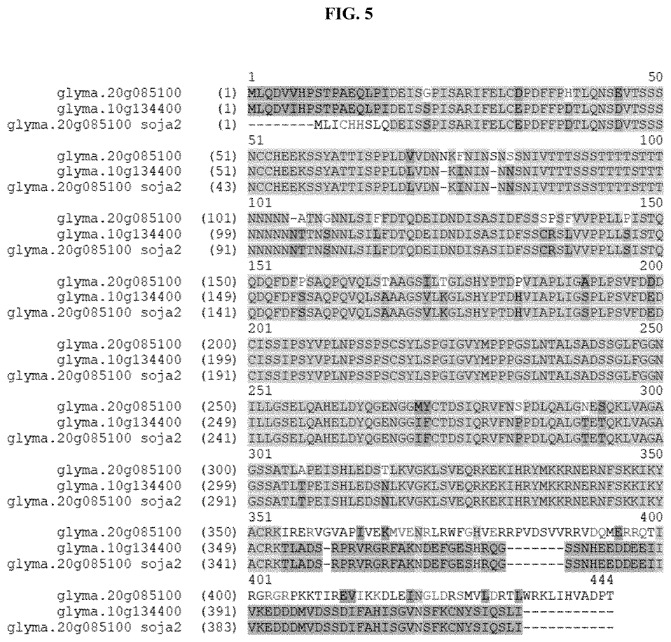

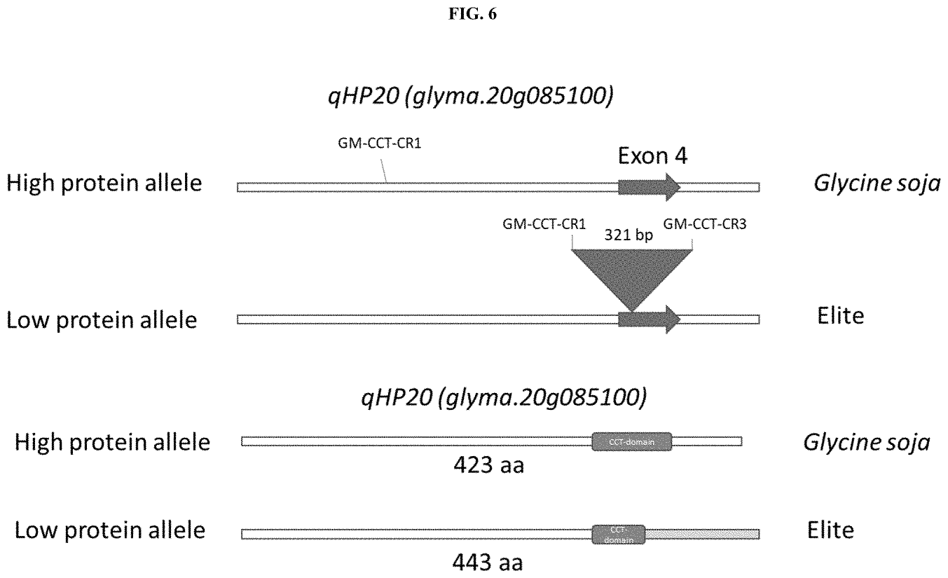

Based on genome sequence analysis of high protein lines and low protein lines, one candidate gene, Glyma.20g085100 (SEQ ID NO:36), has been identified as a potential causative gene for high protein phenotype in the qHP20 region. Compared to high protein Glycine Soja genomic sequences and soybean paralogue glyma.10g134400 (SEQ ID NO: 40), glyma.20g085100 from elite low protein lines, including Williams82, contains a 321 bp insertion in exon 4 which may be the potential causative mutation for the loss of high protein phenotype in the elite soybean (See FIG. 4). This 321 bp insertion is found in all elite low protein lines but not in high protein Danbaekkong and Glycine Soja lines. Glyma.20g850100 encodes a CCT (Constans, Co-like, and TOC1) domain protein. The CCT-domain proteins play an important role in modulating flowering time with pleiotropic effects on morphological traits and stress tolerances in rice, maize, and other cereal crops (Yipu Li and Mingliang Xu, 2017, CCT family genes in cereal crops: A current overview. The Crop Journals 449-458). The function of CCT-domain protein in soybean is unknown. The 321 bp fragment is inserted in the middle of CCT-domain and generates a new open reading frame which produces a completely different 88 amino acids C-terminal (See FIG. 5). The disruption of CCT-domain protein could be non-functional, resulting in low protein content in elite soybean (See FIG. 6). To validate the insertion is the causative mutation for low protein, a pair of guide RNA Gm-CCT-CR2 (SEQ ID NO: 25) and CR3 (SEQ ID NO: 26) are designed to delete the insertion in elite soybean (Table 3). Removal of 321 bp insertion from elite line should restore the function of CCT-domain protein and increase seed protein content. Furthermore, a single guide RNA Gm-CCT CR1 (SEQ ID NO: 24) is targeted to the exon 2 of the glyma.20g850100 to knockout the gene function. Introduction of this gRNA with CAS9 into high protein line should reduce protein content in seeds.

### Example 4. Mapping a Disease QTL with Two Causative Genes in Maize

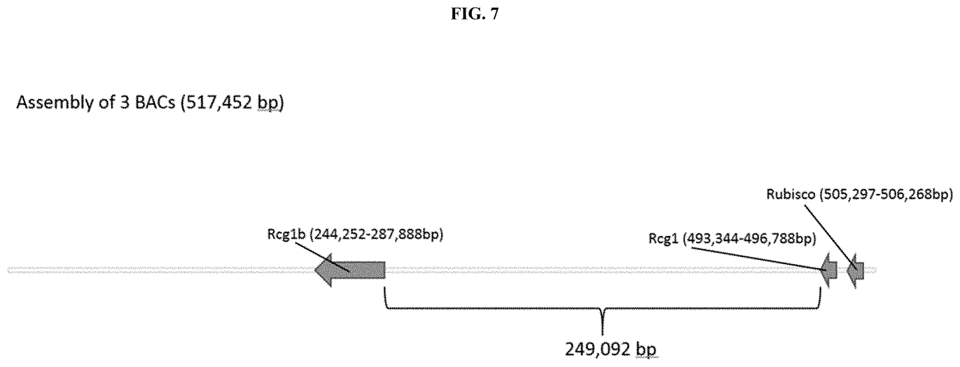

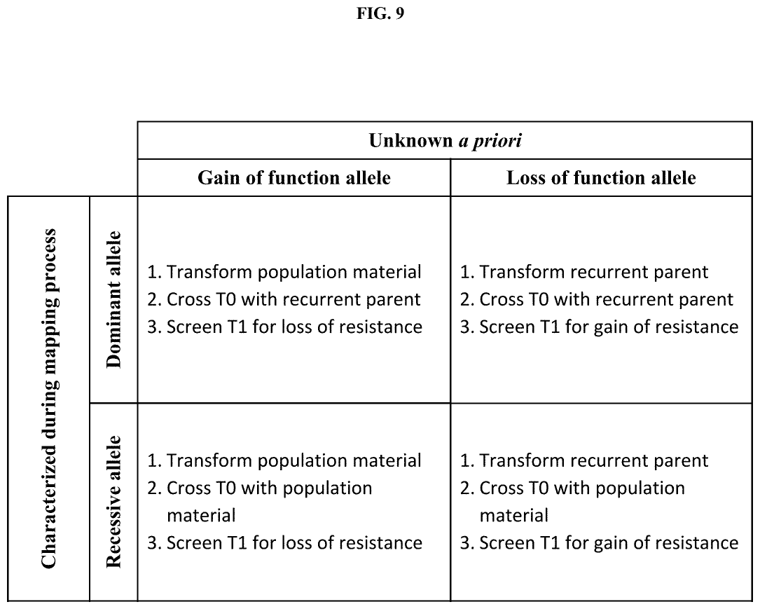

An example of using this method is exemplified by considering Rcg1 (SEQ ID NO: 3 encoded by SEQ ID NO: 1 of U.S. Pat. No. 8,062,847B2, herein incorporated by reference) and Rcg1b (SEQ ID NO: 246 encoded by SEQ ID NO: 245 of U.S. Pat. No. 8,053,631B2, herein incorporated by reference), an NLR gene pair where both genes are required for significant resistance to the hemibiotrophic pathogen Colletotrichum graminicola that causes anthracnose stalk rot in corn. The two genes reside ˜250 kb apart on a rare, large (˜300 kb) non collinear fragment where recombination is not possible with material lacking the fragment (FIG. 7; See also SEQ ID NO: 137 and FIGS. 9(a-b) of U.S. Pat. No. 8,062,847B2, herein incorporated by reference). The editing fine mapping method is used to create edits that delete the rcg1 genomic sequence (3445 bp) and the rcg1b genomic sequence (43637 bp) independently once the resistance gene sequence motifs from the donor have been identified through bioinformatic analysis.

### Fine Mapping Challenged by Lack of Homology Between Mapping Parents

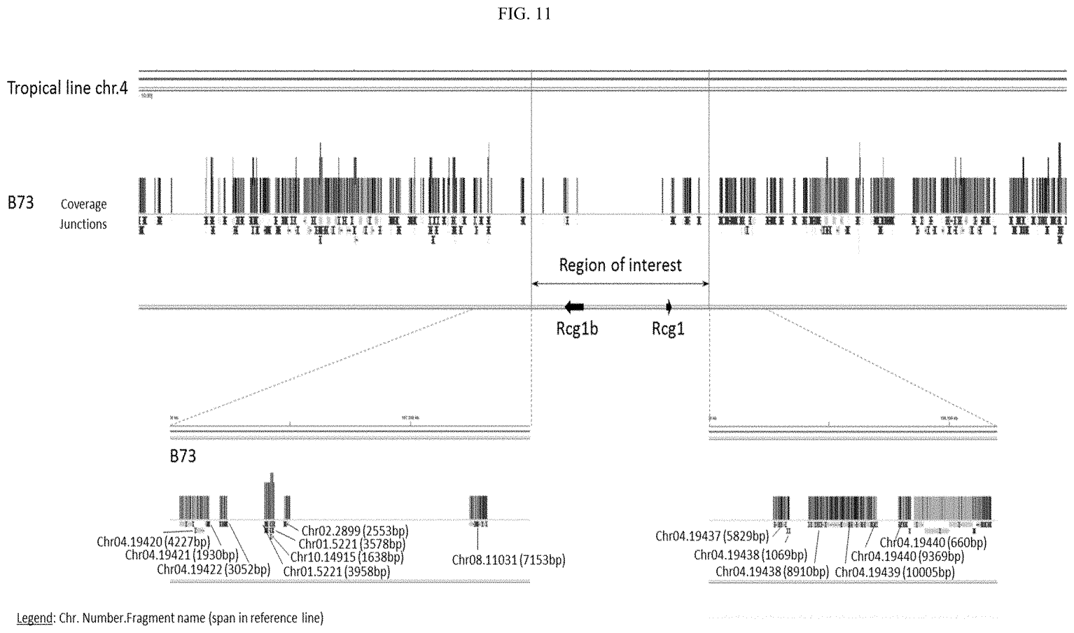

The region of interest corresponds to a ˜500 kb fragment from the resistance donor line, delimited by left and right markers. Large scale sequence alignments between the resistance donor and B73 as an example of North American germplasm revealed a low level of homology in the region of interest and a gradual loss of colinearity on the borders (FIG. 11). Colinearity refers to the succession of homologous fragments in a conserved order. This finding suggested that further fine mapping to narrow down the region of interest was futile, given that sequence homology was one of the prerequisites for the occurrence of meiotic crossing over events.

### CRISPR-Based Fine Mapping Strategy to Elucidate Interval

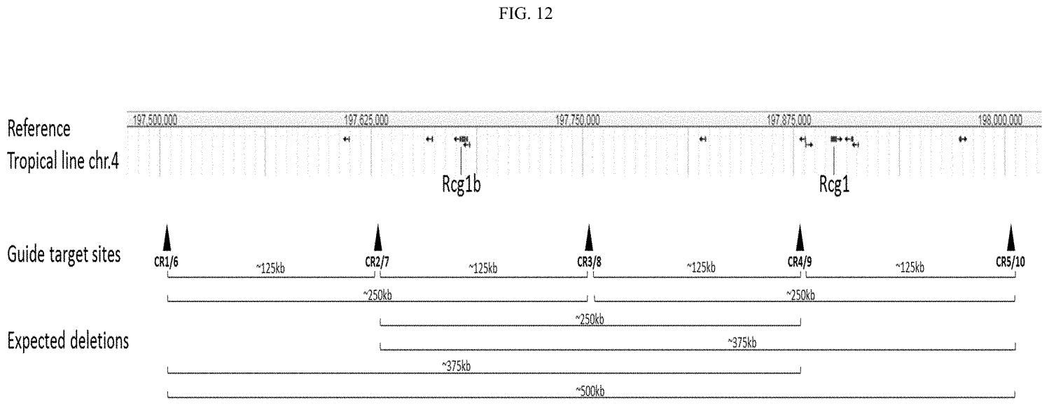

An alternative method is provided here to further narrow the region of interest and identify causal genes. Guide RNAs were designed to produce large deletions in the region of interest (Table 4). Those deletions, in conjunction with the functional annotation of the region of interest, provide the tools to identify causal genes. In this example, deletions are produced that encompass each or both or none of the causal genes (FIG. 12).

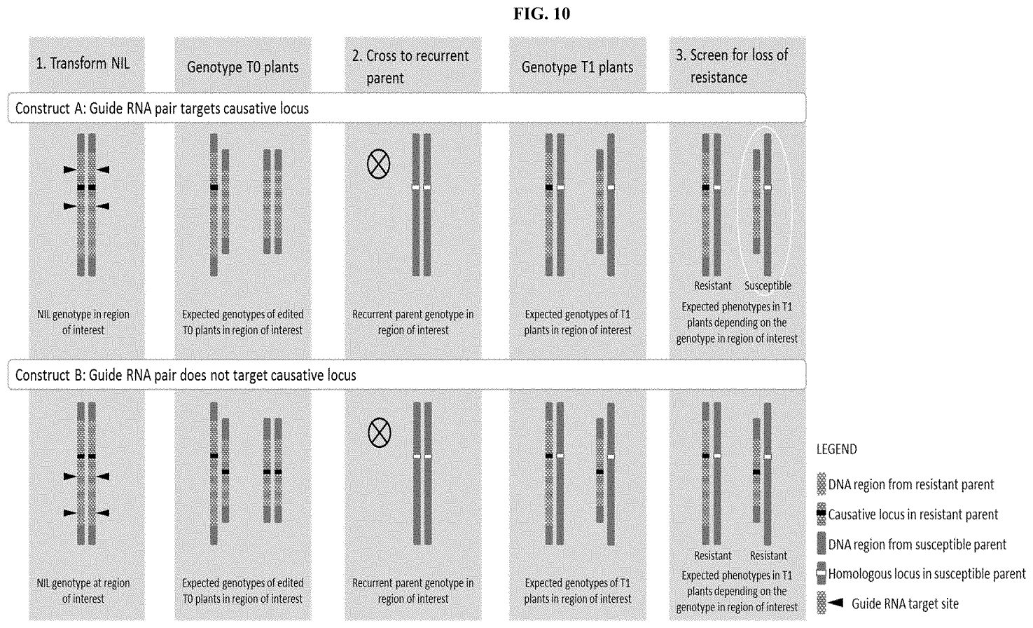

Based on the dominance/recessivity characteristic and loss/gain of function mode of action, an experimental scheme was designed to further map the interval of interest (FIG. 9). During the population development and QTL mapping process, the resistance allele is expected to behave in a dominant fashion. A situation of dominance and gain of function may occur as illustrated in FIG. 10.

Using this strategy, a disease resistant near isogenic line (NIL) is generated during the fine mapping process and is used to create variants with selected deletions within the introgressed region. The deletions encompass the full region of interest and a subset of regions within the region of interest. Deletions may or may not encompass regions predicted to encode genes. Deletions may encompass one or several predicted genes. The deletions in this example range from approximately 125 kbp to approximately 500 kbp.

A series of guide RNA pairs targeting specific sites within the region of interest are designed. When delivered to the cell in combination with Cas9, these guides are expected to produce genomic deletions. At T0, edited plants are selected and genotyped to verify the occurrence of the expected deletion. T0 plants may be edited on a single or both chromosomes, thus respectively hemizygous or homo/heterozygous at the edited locus. To identify edits that encompass the causative locus, the mating scheme involves crossing the T0 plants to the disease susceptible parent used in the population. At T1, plants are genotyped again to verify mendelian segregation of the edited alleles. T1 plants are all expected to contain one copy of the susceptible parental allele and one copy of either the resistant NIL allele or the edited allele.

The resistant allele is expected to be dominant, and most of the T1 plants are expected to display a disease resistant phenotype, with the exception of edited plants specifically containing deletions encompassing the causative locus, which should be susceptible (or less resistant) to the disease (See FIG. 10).

Using this screening scheme, further sequencing and comparison of T1 plants displaying a susceptible versus resistant phenotype is used to identify the causal region or gene.

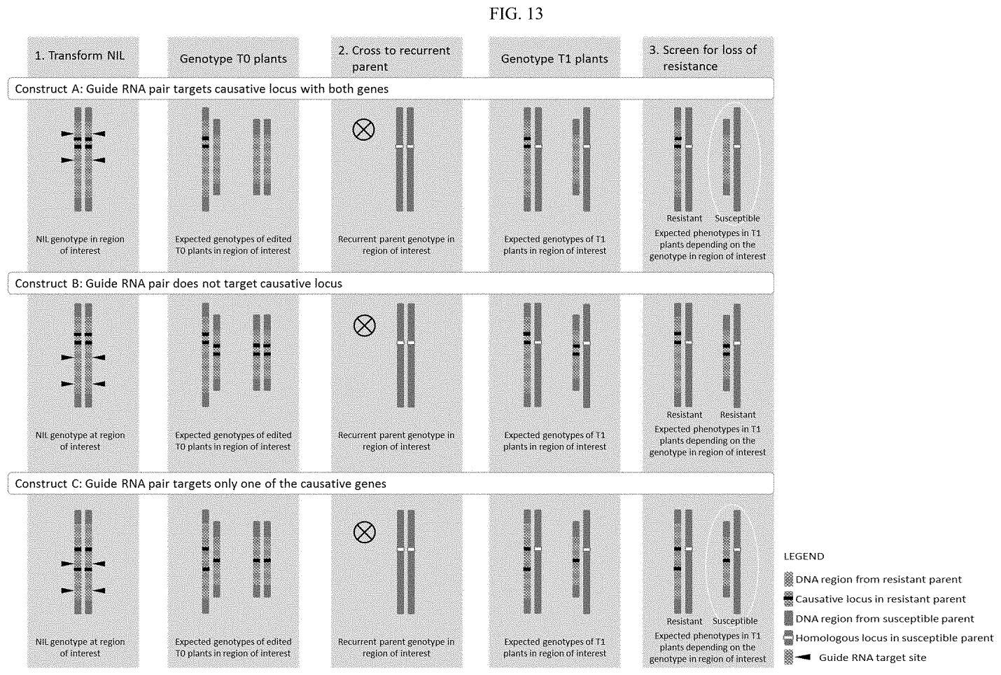

In this example, two genes provide resistance to anthracnose stalk rot: Rcg1b and Rcg1. This method provides the means to elucidate this mode of action (FIG. 13).

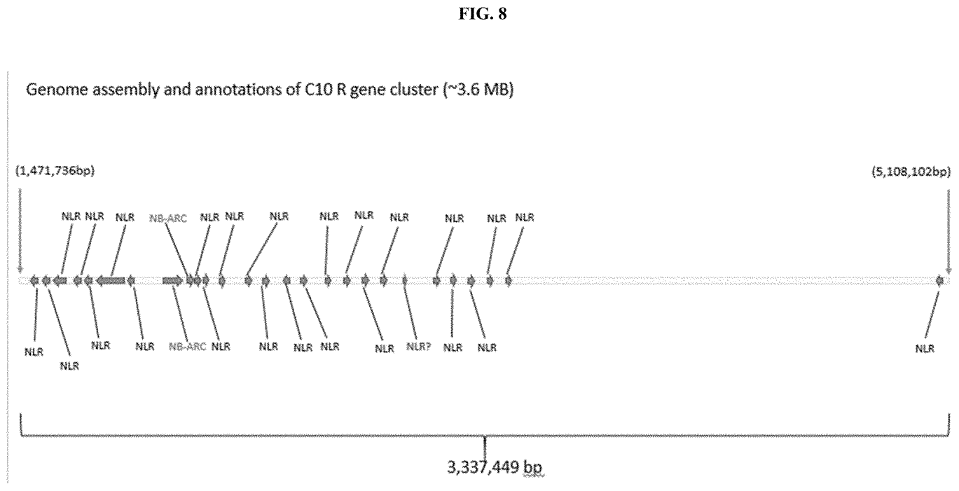

The method described here allows to further elucidate complex regions where more than one protein coding gene may be at play in contributing to a QTL or it is extremely difficult to isolate genes in a cluster via recombination (See FIG. 8). The assembly is from the known disease resistance gene cluster (an “R gene cluster”) on the short arm of chromoseome 10, and contains about 26 genes of varying degree of similarity to each other, all in close proximity. Deleting the genes or a subset of them delimited by recombination allows isolation of the causative genes.

TABLE 4
guide RNAs designed to produce deletions in the anthracnose
stalk rot resistance QTL region of interest.

| | Approx- | | | | |
|---|---|---|---|---|---|
| | imate | | Guide | | Guide |
| Edit | Expected | | 1 | | 2 |
| Designation | Deletion | Guide 1 | SEQ ID | Guide 2 | SEQ ID |
| (Guide Pair) | Size (Bp) | Name | NO: | Name | NO: |
| ZM-CR1 + 2 | 125,104 | ZM-CR1 | 1 | ZM-CR2 | 2 |
| ZM-CR2 + 3 | 125,058 | ZM-CR2 | 2 | ZM-CR3 | 3 |
| ZM-CR3 + 4 | 124,460 | ZM-CR3 | 3 | ZM-CR4 | 4 |
| ZM-CR4 + 5 | 126,162 | ZM-CR4 | 4 | ZM-CR5 | 5 |
| ZM-CR1 + 3 | 250,162 | ZM-CR1 | 1 | ZM-CR3 | 3 |
| ZM-CR3 + 5 | 250,622 | ZM-CR3 | 3 | ZM-CR5 | 5 |
| ZM-CR2 + 4 | 249,518 | ZM-CR2 | 2 | ZM--CR4 | 4 |
| ZM-CR1 + 4 | 374,622 | ZM-CR1 | 1 | ZM-CR4 | 4 |
| ZM-CR2 + 5 | 375,680 | ZM-CR2 | 2 | ZM-CR5 | 5 |
| ZM-CR1 + 5 | 500,784 | ZM-CR1 | 1 | ZM-CR5 | 5 |
| ZM-CR6 + 7 | 125,632 | ZM-CR6 | 6 | ZM-CR7 | 7 |
| ZM-CR7 + 8 | 124,754 | ZM-CR7 | 7 | ZM-CR8 | 8 |
| ZM-CR8 + 9 | 126,256 | ZM-CR8 | 8 | ZM-CR9 | 9 |
| ZM-CR9 + 10 | 124,381 | ZM--CR9 | 9 | ZM-CR10 | 10 |
| ZM-CR6 + 8 | 250,386 | ZM-CR6 | 6 | ZM-CR8 | 8 |
| ZM-CR8 + 10 | 250,637 | ZM-CR8 | 8 | ZM-CR10 | 10 |

### Example 5. Fine Mapping Scenario for a Maize QTL

Populations are developed to identify a chromosome QTL contributing to a desired trait. The resistance donor is a diverse source containing desired trait with a large effect size in comparison to the elite germplasm to be improved. A well characterized temperate line is used as a recurrent parent. Initial QTL discovery is done in a test cross population ((diverse source line x temperate line) x tester) with ˜200 individuals. A significant QTL is found in this population, mapping to a single interval. This effect is then validated in the same population or others using the same source and new elites (diverse line x elite inbreds). The validation populations or the original ones are then selected for recombinant screening to search for recombinants in the region and development of NILs with the donor fragment across the QTL interval.

### Fine Mapping Challenged by Lack of Homology Between Mapping Parents

Using recombinants and field phenotyping at single or multiple locations, the QTL is fine mapped to a small genetic interval on a chromosome. Fine mapping further narrows the interval to a small region flanked by markers that can be uniquely mapped to a known contiguous sequence from the elite line. In the diverse resistance donor, this region of interest corresponds to this physical interval.

Although many recombinants are screened, no recombinant are expected to be recovered inside the region, preventing further narrowing of the interval of interest.

The full diverse resistant donor genome sequence is determined. Marker data show that the elite sequence is not identical in the interval of interest, but collinearity is generally assumed for those two inbreds. Using the diverse resistance donor as a reference, 10 kb fragments of the elite genome are aligned and assigned to their best matching location in the diverse resistance donor genome. While most fragments are expected to align to their homologous region in the diverse resistance donor and display a high level of synteny with the elite line, some fragments are expected to be inverted, rearranged, or only partially aligned, suggesting large structural differences between the two genomes. In addition, regions with few to no match in the elite line are expected to be observed as well, indicating that some regions are unique to the diverse resistance donor genome. This may be evident within the region of interest. Additional inbred lines are also inspected and expected to display a similar pattern. Altogether these observations suggest that the region of interest in the diverse resistance donor may share a very low level of sequence homology with other inbred lines.

Sequence homology is one of the prerequisites for the occurrence of meiotic crossing over events. The expected results show a lack of recombination events in the region of interest during the fine mapping process. The expected results show that further pursuing this approach by screening additional progeny is unlikely to yield useful recombinants.

### CRISPR-Based Fine Mapping Strategy to Elucidate Interval

Based on the dominance/recessivity characteristic and loss/gain of function mode of action, an experimental scheme is designed to further map the interval of interest (FIG. 9). During the population development and QTL mapping process, the resistance allele is expected to behave in a dominant or semi-dominant fashion. A situation of dominance and gain of function may occur as illustrated in FIG. 10.

Using this strategy, a disease resistant near isogenic line (NIL) is generated during the fine mapping process and is used to create variants with selected deletions within the introgressed region. The deletions may be encompassing the full region of interest or a subset of regions within the region of interest. These smaller deletions may encompass targeted areas such as gene-rich regions, or regions containing clusters of disease resistance genes, or regions of major structural variation, or regions of higher gene expression. These deletions may be ranging from kbp to several Mbp. These deletions may be designed to overlap or not.

A series of guide RNA pairs targeting specific sites within the region of interest are designed. When delivered to the cell in combination with Cas9, these guides are expected to produce genomic deletions. At T0, edited plants are selected and genotyped to verify the occurrence of the expected deletion. T0 plants may be edited on a single or both chromosomes, thus respectively hemizygous or homo/heterozygous at the edited locus. To identify edits that encompass the causative locus, the mating scheme involves crossing the T0 plants to the disease susceptible parent used in the population. At T1, plants are genotyped again to verify mendelian segregation of the edited alleles. T1 plants are all expected to contain one copy of the susceptible parental allele and one copy of either the resistant NIL allele or the edited allele.

The resistant allele is expected to be dominant or semi-dominant, and most of the T1 plants are expected to display a disease resistant phenotype, with the exception of edited plants specifically containing deletions encompassing the causative locus, which should be susceptible (or less resistant) to the disease (See FIG. 10).

Using this screening scheme, further sequencing and comparison of T1 plants displaying a susceptible versus resistant phenotype is used to identify the causal region or gene.

1. A method for fine mapping a desired trait comprising:
a) introducing a site-specific modification in at least one target site in an endogenous genomic locus in a plant;
b) obtaining the plant having a modified nucleotide sequence; and
c) screening for the site-specific modification; and
d) screening for an increase or decrease in a phenotype of the desired trait.

2. The method of claim 1, further comprising introducing at least a second site-specific modification in the endogenous genomic locus, wherein said site-specific modification comprises at least one nucleic acid deletion, insertion, or polymorphism compared to the endogenous genomic sequence, allele, or genomic locus.

3. The method of claim 1, wherein the site-specific modification is induced by a nuclease selected from the group consisting of: a TALEN, a meganuclease, a zinc finger nuclease, and a CRISPR-associated nuclease.

4. The method of claim 1, wherein said method further comprises selecting a plant having the modified nucleotide sequence.

5. The method of claim 1, wherein the endogenous genomic locus is located within a known QTL.

6. The method of claim 5, wherein the genomic locus is at least partially sequenced, and wherein the site-specific modification occurs within the at least partially sequenced genomic locus.

7. The method of claim 1, wherein the endogenous genomic locus encompasses a random mutation fine-mapping.

8. The method of claim 1, wherein the plant exhibits either increased or decreased disease resistance.

9. The method of claim 1, wherein the plant either increased or decreased soybean protein concentration.

10. The method of claim 1, wherein the plant either increased or decreased grain yield, plant health, stature, stalk strength, or pest resistance.

11. The method of claim 1, wherein said site-specific modification comprises a deletion, INDEL, or SNP in a non-coding region of the endogenous genomic locus.

12. The method of claim 11, wherein the non-coding region comprises a promoter, an intron, or an untranslated region.

13. The method of claim 1, wherein the site-specific modification comprises a deletion, INDEL, or SNP in the coding region of a gene of interest.

14. The method of claim 1, wherein the site-specific modification comprises a deletion, INDEL, or SNP in the promoter or coding region of one or more QTL phenotype causal genes.

15. The method of claim 1, wherein the at least one site-specific modification comprises at least one double strand break introduced at one or multiple target sites by a Cas9 endonuclease.

16. The method of claim 15, wherein Cas9 endonuclease is guided by at least one guide RNA.

17. The method of claim 16, wherein the at least one guide RNA directs a site-specific modification at one or several specific target sites within the endogenous genomic locus.

18. The method of claim 1, wherein the endogenous genomic locus has a low intrinsic recombination frequency.

19. The method of claim 18, wherein the endogenous genomic locus is a centromeric region.

20. The method of claim 1, wherein the endogenous genomic locus represents a unique haplotype that cannot be recombined with other haplotypes within the same interval.

21. The method of claim 20, wherein the unique haplotype cannot be recombined with other haplotypes due to lack of homology.

22. A method for identifying a causal gene of a desired trait comprising:
a) introducing at least one site-specific modification in an endogenous genomic locus in a plant;
b) obtaining the plant having at least one site-specific modification;
c) screening the plant or the plant's progeny for the presence or absence of the desired trait; and
d) identifying the causal gene.

23. The method of claim 22, further comprising identifying one or more linked genes responsible for the desired trait and functionally affected by the targeted modification.

24. The method of claim 22, wherein the at least one site-specific modification is a deletion, INDEL, or SNP.

25. The method of claim 24, wherein the deletion comprises a sequence comprising more than one gene.

26. The method of claim 22, further comprising introducing a large specific deletion wherein a double stranded break occurs at the first target site and a second target site located on the same chromosome as the first target site.

27. The method of claim 24, wherein the at least one deletion comprises a sequence comprising the an entire known QTL for the desired trait.

28. A method to create a novel haplotype in a genomic locus comprising:
a) introducing at least one site-specific modification in an endogenous genomic locus in a first plant;
b) screening for the site-specific modification; and
c) correlating the haplotype with a phenotype to establish a cause and effect relationship between the at least one site-specific modification and the desired trait.

29. The method of claim 28, further comprising introducing at least a second site-specific modification in the endogenous genomic locus, wherein said site-specific modification comprises at least one nucleic acid deletion, insertion, or polymorphism compared to the endogenous genomic sequence, allele, or genomic locus.

30. The method of claim 28, wherein the site-specific modification is induced by a nuclease selected from the group consisting of: a TALEN, a meganuclease, a zinc finger nuclease, and a CRISPR-associated nuclease.

31. The method of claim 28, wherein said method further comprises selecting a plant having a modified nucleotide sequence.

32. The method of claim 28, wherein the endogenous genomic locus is located within a known QTL.

33. The method of claim 32, wherein the genomic locus is at least partially sequenced, and wherein the site-specific modification occurs within the at least partially sequenced genomic locus.

34. The method of claim 28, wherein the endogenous genomic locus encompasses a random mutation fine-mapping.

35. The method of claim 28, wherein the at least one site-specific modification comprises at least one double strand break introduced at the one or multiple target sites by a Cas9 endonuclease.

36. The method of claim 35, wherein Cas9 endonuclease is guided by at least one guide RNA.

37. The method of claim 36, wherein the at least one guide RNA directs a site-specific modification at one or several specific target sites within the endogenous genomic locus.

38. The method of claim 28, wherein the endogenous genomic locus has a low intrinsic recombination frequency.

39. The method of claim 28, wherein the endogenous genomic locus is a centromeric region.

40. The method of claim 28, wherein the endogenous genomic locus represents a unique haplotype that cannot be recombined with other haplotypes within the same interval.

41.-79. (canceled)
